SEQUENCE LISTING (4 sequences)

SEQ ID NO: 1 - protein, 56 aa, Synthetic construct
GERPFFCNQCGASFTQKGNLLRHIKLHSGEKPFKCHLCNYACRRRDALTGHLRTHS

SEQ ID NO: 2 - protein, 509 aa, Homo sapiens
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 3 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 4 - protein, 526 aa, Homo sapiens [sequence omitted]

(19) United States
(12) Patent Application Publication
(10) Pub. No.: US 2024/0327381 A1
(43) Pub. Date: Oct. 3, 2024

(54) Compounds and Compositions for Selective Degradation of Engineered Proteins

(71) Applicants: Celgene Corporation, Princeton, NJ (US); Bristol-Myers Squibb Company, Princeton, NJ (US)

(72) Inventors: Laura Akullian D'Agostino, Sudbury, MA (US); Godwin Kumi, Princeton, NJ (US); Haibo Liu, Lexington, MA (US)

(73) Assignees: Celgene Corporation, Princeton, NJ (US); Bristol-Myers Squibb Company, Princeton, NJ (US)

(21) Appl. No.: 18/435,117

(22) Filed: Feb. 7, 2024

Related U.S. Application Data
(60) Provisional application No. 63/444,208, filed on Feb. 8, 2023.

(51) Int. Cl. C07D 401/14; A61K 31/454; A61K 31/4545; A61K 31/4709; A61K 31/4725; A61K 31/496; A61K 31/498; C07D 401/04; C07D 405/14; C07D 409/14; C07D 413/14; C07D 417/14; C07D 451/00; C07D 471/04

(52) U.S. Cl. CPC C07D 401/14; A61K 31/454; A61K 31/4545; A61K 31/4709; A61K 31/4725; A61K 31/496; A61K 31/498; C07D 401/04; C07D 405/14; C07D 409/14; C07D 413/14; C07D 417/14; C07D 451/00; C07D 471/04

(57) ABSTRACT

Provided herein are compounds and compositions thereof for degrading an engineered polypeptide in a cell. In some embodiments, the compounds and compositions are provided for treatment of cancer.

### CROSS-REFERENCE TO RELATED APPLICATIONS

This application claims the benefit of priority of U.S. Provisional Application No. 63/444,208, filed Feb. 8, 2023, which is incorporated by reference herein in its entirety for any purpose.

### SEQUENCE LISTING

The present application contains a Sequence Listing, which has been submitted electronically in XML format. Said XML copy, created on Jan. 23, 2024, is named “01277-0037-00PCT.xml” and is 6,097 bytes in size. The information in the electronic format of the sequence listing is incorporated herein by reference in its entirety.

### FIELD

The present disclosure relates to compounds and compositions thereof for degrading engineered proteins in cells.

### BACKGROUND

Engineered cells comprising an engineered, heterologous polypeptide, such as chimeric antigen receptor T (CAR-T) cells, have been developed for therapeutic use. Modulation of the expression levels of such engineered, heterologous polypeptides may improve the therapeutic benefit of the engineered cells by, for example, decreasing side effects and/or increasing efficacy of the engineered cells.

Accordingly, in one aspect, provided herein are engineered polypeptides and degradation agents, wherein the engineered polypeptides comprise a degradation domain that mediates ubiquitination in cell when the degradation domain binds to a degradation agent.

### SUMMARY

Described herein, in certain embodiments, are compounds and compositions thereof for modulating levels of a heterologous polypeptide in a cell. In various embodiments, the compounds and compositions thereof may be used to decrease the level of the heterologous polypeptide in the cell.

The present embodiments can be understood more fully by reference to the detailed description and examples, which are intended to exemplify non-limiting embodiments.

In some embodiments, provided herein are compounds of Formula (I):

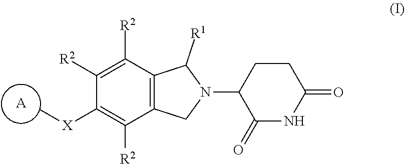

or a pharmaceutically acceptable salt thereof, wherein:
R¹ is H or oxo;
each R² is independently H or halo, wherein at least one R² is fluoro;
X is bond, C₁-C₃ alkylene, —C(O)NHCH₂—, —NHC(O)—, —C(O)—, or —(C₁-C₃ alkylene)NH(C₁-C₃ alkylene)-;
   Ring A is optionally substituted C₅-C₆ cycloalkyl, optionally substituted C₅-C₆ aryl, optionally substituted 6-10 membered heterocyclyl, or optionally substituted 5-9 membered heteroaryl, wherein heterocyclyl or heteroaryl contains 1-3 heteroatoms selected from nitrogen, oxygen, and sulfur.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5%, or ±1% of the indicated range, value, or structure, unless otherwise indicated.

“Amino” refers to the —NH₂ radical.

“Cyano” refers to the —CN radical.

“Oxo” refers to the ═O radical.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)—CH₂, —C(CH₃)—CH(CH₃), —C(CH₂CH₃)—CH₂, —C═CH, —C═C(CH₃), —C═C(CH₂CH₃), —CH₂C═CH, —CH₂C═C(CH₃) and —CH₂C═C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkylene” group refers to the same residues as alkyl, but having bivalency. Particular alkylene groups are those having from 1 to 10 carbon atoms (C₁-C₁₀ alkylene), typically from 1 to 8 carbons (C₁-C₈ alkylene) or, in some embodiments, from 1 to 6 (C₁-C₆ alkylene) or 1 to 3 (C₁-C₃ alkylene) carbon atoms. Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₈ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2 (1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heterocyclylene” group refers to a divalent “heterocyclyl” group.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1 (2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

When the groups described herein are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag Gmbh & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

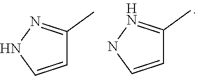

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an IRAK3 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

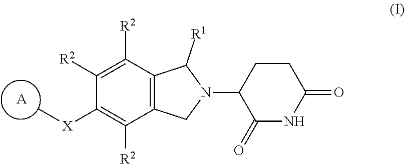

or a pharmaceutically acceptable salt thereof, wherein:
R¹ is H or oxo;
each R² is independently H or halo, wherein at least one R² is fluoro;
X is bond, C₁-C₃ alkylene, —C(O)NHCH₂—, —NHC(O)—, —C(O)—, or —(C₁-C₃ alkylene)NH(C₁-C₃ alkylene)-;
   Ring A is optionally substituted C₅-C₆ cycloalkyl, optionally substituted C₅-C₆ aryl, optionally substituted 6-10 membered heterocyclyl, or optionally substituted 5-9 membered heteroaryl, wherein heterocyclyl or heteroaryl contains 1-3 heteroatoms selected from nitrogen, oxygen, and sulfur.

In some embodiments, R¹ is H or oxo. In some embodiments, R¹ is H. In some embodiments, R¹ is oxo.

In some embodiments, R² is H or halo. In some embodiments, R² is H. In some embodiments, R² is halo. In some embodiments, R² is F, Cl, or Br. In some embodiments, R² is F.

In some embodiments, X is bond, C₁-C₃ alkylene, —C(O)NHCH₂—, —NHC(O)—, —C(O)—, or —(C₁-C₃ alkylene)NH(C₁-C₃ alkylene)-. In some embodiments, X is bond, C₁ alkylene, —C(O)NHCH₂—, —NHC(O)—, —C(O)—, or —(C₁ alkylene)NH(C₁ alkylene)-. In some embodiments, X is bond, —CH₂—, —C(O)NHCH₂—, —NHC(O)—, —C(O)—, or —CH₂NHCH₂—.

In some embodiments, X is bond.

In some embodiments, X is C₁-C₃ alkylene. In some embodiments, X is C₁ alkylene. In some embodiments, X is —CH₂—.

In some embodiments, X is —C(O)NHCH₂—.

In some embodiments, X is —NHC(O)—.

In some embodiments, X is —C(O)—.

In some embodiments, X is —(C₁-C₃ alkylene)NH(C₁-C₃ alkylene)-. In some embodiments, X is —(C₁ alkylene)NH(C₁ alkylene)-. In some embodiments, X is —CH₂NHCH₂—.

In some embodiments, Ring A is optionally substituted C₅-C₆ cycloalkyl, optionally substituted C₅-C₆ aryl, optionally substituted 6-10 membered heterocyclyl, or optionally substituted 5-9 membered heteroaryl, wherein heterocyclyl or heteroaryl contains 1-3 heteroatoms selected from nitrogen, oxygen, and sulfur. In some embodiments, Ring A is optionally substituted C₆ cycloalkyl, optionally substituted C₆ aryl, optionally substituted 6-10 membered heterocyclyl, or 5-9 membered heteroaryl optionally substituted with optionally substituted C₁-C₃ alkyl, optionally substituted amine, or C₄-C₆ cycloalkyl, wherein heterocyclyl or heteroaryl contains 1-3 heteroatoms selected from nitrogen, oxygen, and sulfur. In some embodiments, Ring A is optionally substituted cyclohexyl, optionally substituted phenyl, 6-10 membered heterocyclyl substituted with H, OH, cyano, halo, optionally substituted C₁-C₃ alkyl, optionally substituted C₃-C₆ cycloalkyl, —C(O)(6-membered heteroaryl), or —C(O)(9-membered heterocyclyl), or 5-9 membered heteroaryl optionally substituted with C₁-C₃ alkyl, amine, or C₄ cycloalkyl, wherein heterocyclyl or heteroaryl contains 1-3 heteroatoms selected from nitrogen, oxygen, and sulfur. In some embodiments, Ring A is cyclohexyl substituted with —CF₃, phenyl substituted with —CHF₂, 6-10 membered heterocyclyl substituted with H, OH, cyano, F, Cl, Br, optionally substituted C₁-C₃ alkyl, optionally substituted C₄-C₆ cycloalkyl, —C(O)(6-membered heteroaryl), or —C(O)(9-membered heterocyclyl), or 5-9 membered heteroaryl optionally substituted with C₁ alkyl, amine, or C₄ cycloalkyl, wherein heterocyclyl or heteroaryl contains 1-3 heteroatoms selected from nitrogen, oxygen, and sulfur. In some embodiments, Ring A is cyclohexyl substituted with —CF₃, phenyl substituted with —CHF₂, 6-10 membered heterocyclyl substituted with H, OH, cyano, F, Cl, optionally substituted C₁-C₃ alkyl, C₄-C₆ cycloalkyl, —C(O)(6-membered heteroaryl), or —C(O)(9-membered heterocyclyl), or 5-9 membered heteroaryl optionally substituted with —CH₃, amine, or cyclobutyl, wherein heterocyclyl or heteroaryl contains 1-3 heteroatoms selected from nitrogen, oxygen, and sulfur.

In some embodiments, Ring A is optionally substituted C₅-C₆ cycloalkyl. In some embodiments, Ring A is optionally substituted C₆ cycloalkyl. In some embodiments, Ring A is optionally substituted cyclohexyl. In some embodiments, Ring A is cyclohexyl substituted with —CF₃. In some embodiments, Ring A is

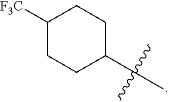 [SMILES: *C1CCC(C(F)(F)F)CC1]

In some embodiments, Ring A is optionally substituted C₅-C₆ aryl. In some embodiments, Ring A is optionally substituted C₆ aryl. In some embodiments, Ring A is optionally substituted phenyl. In some embodiments, Ring A is phenyl substituted with —CHF₂. In some embodiments, Ring A is

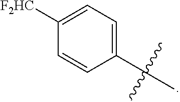 [SMILES: *c1ccc(C(F)F)cc1]

In some embodiments, Ring A is optionally substituted 6-10 membered heterocyclyl that contains 1-3 heteroatoms selected from nitrogen, oxygen, and sulfur. In some embodiments, Ring A is 6-10 membered heterocyclyl substituted with H, OH, cyano, halo, optionally substituted C₁-C₃ alkyl, optionally substituted C₃-C₆ cycloalkyl, —C(O)(6-membered heteroaryl), or —C(O)(9-membered heterocyclyl), wherein heterocyclyl or heteroaryl contains 1-3 heteroatoms selected from nitrogen, oxygen, and sulfur. In some embodiments, Ring A is 6-10 membered heterocyclyl substituted with H, OH, cyano, F, Cl, Br, optionally substituted C₁-C₃ alkyl, optionally substituted C₄-C₆ cycloalkyl, —C(O)(6-membered heteroaryl), or —C(O)(9-membered heterocyclyl), wherein heterocyclyl or heteroaryl contains 1-3 heteroatoms selected from nitrogen, oxygen, and sulfur. In some embodiments, Ring A is 6-10 membered heterocyclyl substituted with H, OH, cyano, F, Cl, optionally substituted C₁-C₃ alkyl, C₄-C₆ cycloalkyl, —C(O)(6-membered heteroaryl), or —C(O)(9-membered heterocyclyl), wherein heterocyclyl or heteroaryl contains 1-3 heteroatoms selected from nitrogen, oxygen, and sulfur. In some embodiments, Ring A is

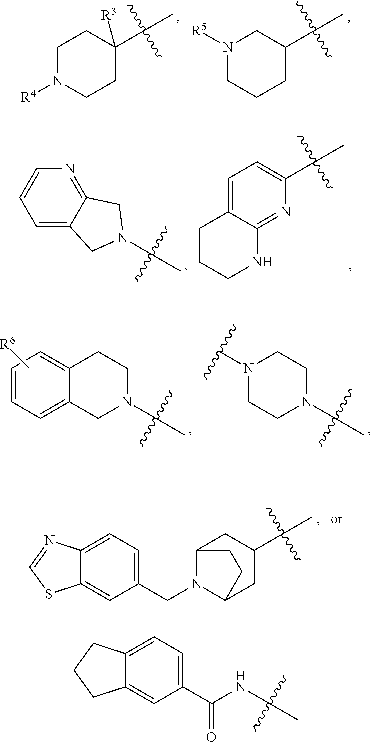

wherein R³ is H or OH; R⁴ is optionally substituted C₁-C₃ alkyl, optionally substituted C₃-C₆ cycloalkyl, —C(O)(6-membered heteroaryl), or —C(O)(9-membered heterocyclyl); R⁵ is optionally substituted C₁-C₃ alkyl; and R⁶ is cyano or halo. In some embodiments, Ring A is

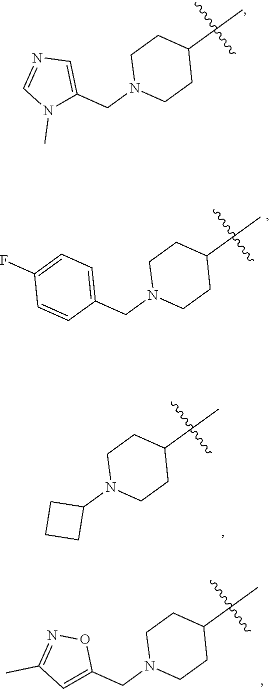 [SMILES: *C1CCN(Cc2cncn2C)CC1 ; *C1CCN(Cc2ccc(F)cc2)CC1 ; *C1CCN(C2CCC2)CC1 ; *C1CCN(Cc2cc(C)no2)CC1]

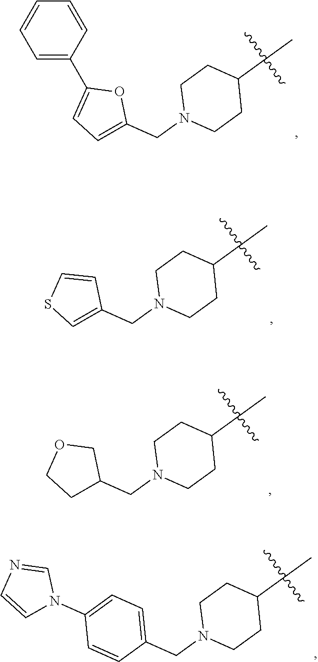 [SMILES: *C1CCN(Cc2ccc(-c3ccccc3)o2)CC1 ; *C1CCN(Cc2ccsc2)CC1 ; *C1CCN(CC2CCOC2)CC1 ; *C1CCN(Cc2ccc(-n3ccnc3)cc2)CC1]

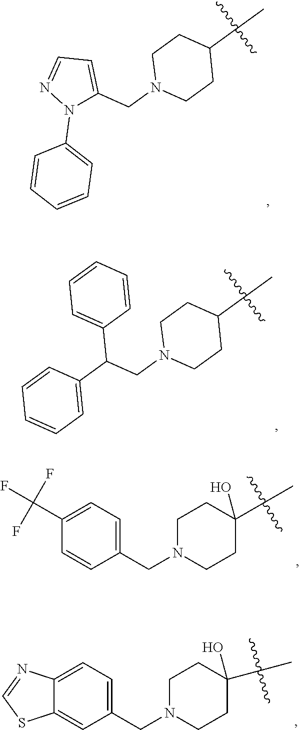

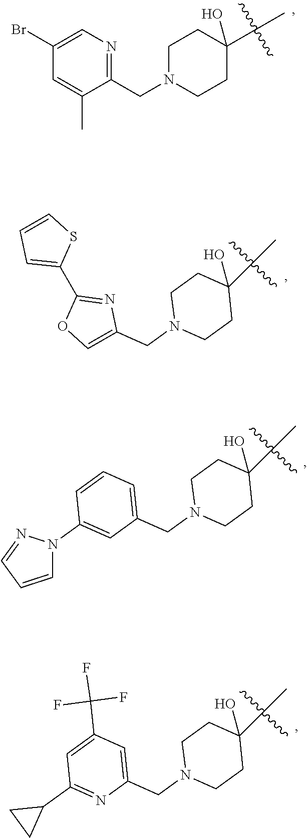 [SMILES: *C1(O)CCN(Cc2ncc(Br)cc2C)CC1 ; *C1(O)CCN(Cc2coc(-c3cccs3)n2)CC1 ; *C1(O)CCN(Cc2cccc(-n3cccn3)c2)CC1 ; *C1(O)CCN(Cc2cc(C(F)(F)F)cc(C3CC3)n2)CC1]

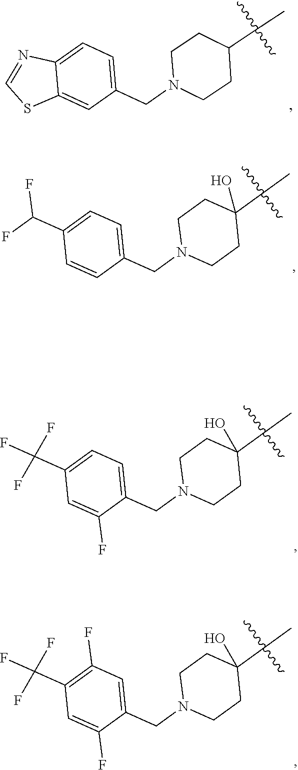 [SMILES: *C1CCN(Cc2ccc3ncsc3c2)CC1 ; *C1(O)CCN(Cc2ccc(C(F)F)cc2)CC1 ; *C1(O)CCN(Cc2ccc(C(F)(F)F)cc2F)CC1 ; *C1(O)CCN(Cc2cc(F)c(C(F)(F)F)cc2F)CC1]

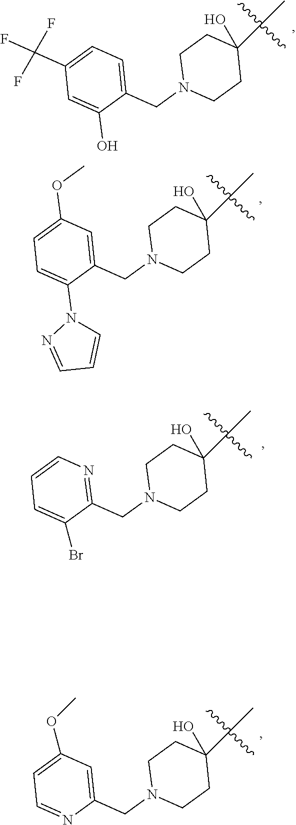

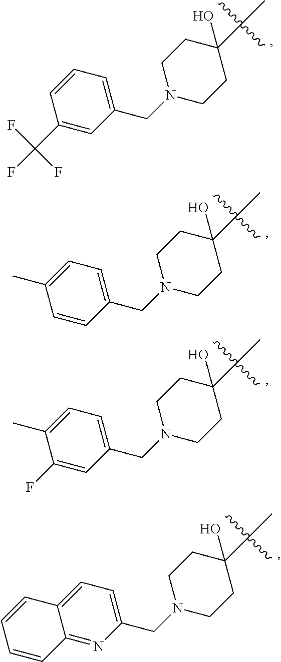 [SMILES: *C1(O)CCN(Cc2cccc(C(F)(F)F)c2)CC1 ; *C1(O)CCN(Cc2ccc(C)cc2)CC1 ; *C1(O)CCN(Cc2ccc(C)c(F)c2)CC1 ; *C1(O)CCN(Cc2ccc3ccccc3n2)CC1]

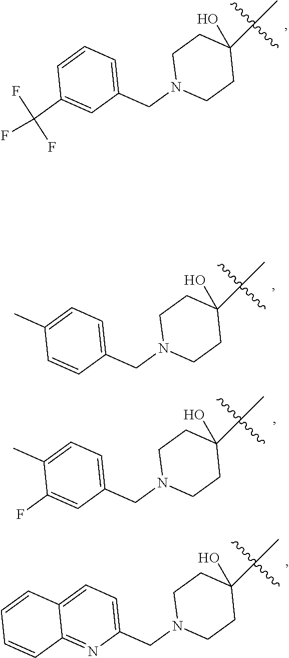 [SMILES: *C1(O)CCN(Cc2cccc(C(F)(F)F)c2)CC1 ; *C1(O)CCN(Cc2ccc(C)cc2)CC1 ; *C1(O)CCN(Cc2ccc(C)c(F)c2)CC1 ; *C1(O)CCN(Cc2ccc3ccccc3n2)CC1]

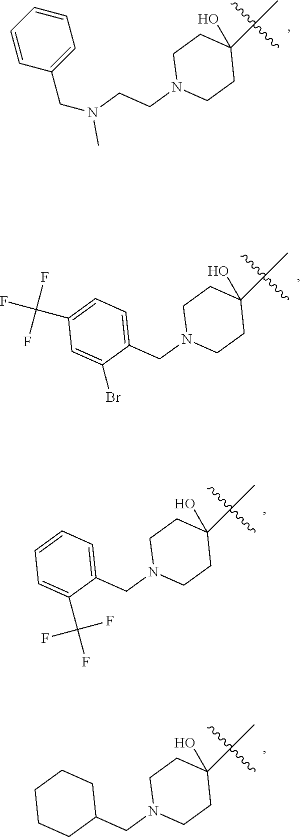 [SMILES: *C1(O)CCN(CCN(C)Cc2ccccc2)CC1 ; *C1(O)CCN(Cc2ccc(C(F)(F)F)cc2Br)CC1 ; *C1(O)CCN(Cc2ccccc2C(F)(F)F)CC1 ; *C1(O)CCN(CC2CCCCC2)CC1]

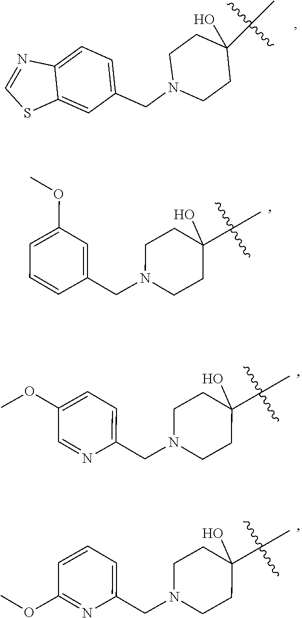 [SMILES: *C1(O)CCN(Cc2ccc3ncsc3c2)CC1 ; *C1(O)CCN(Cc2cccc(OC)c2)CC1 ; *C1(O)CCN(Cc2ccc(OC)cn2)CC1 ; *C1(O)CCN(Cc2cccc(OC)n2)CC1]

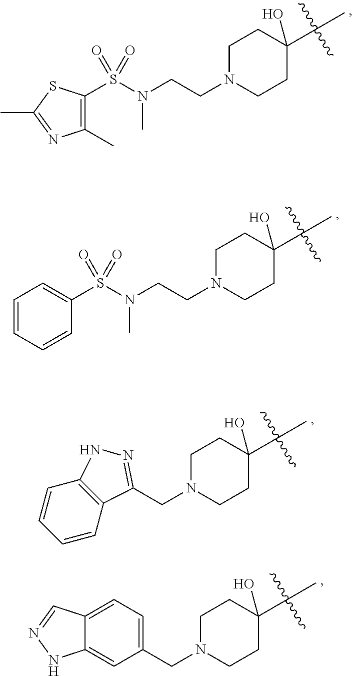 [SMILES: *C1(O)CCN(CCN(C)S(=O)(=O)c2sc(C)nc2C)CC1 ; *C1(O)CCN(CCN(C)S(=O)(=O)c2ccccc2)CC1 ; *C1(O)CCN(Cc2n[nH]c3ccccc23)CC1 ; *C1(O)CCN(Cc2ccc3cn[nH]c3c2)CC1]

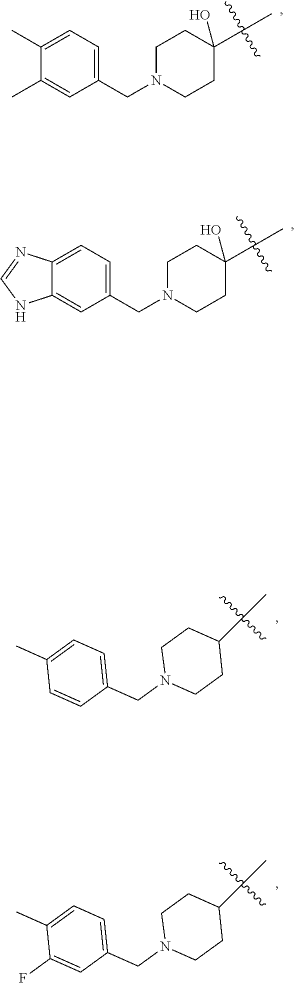 [SMILES: *C1(O)CCN(Cc2ccc(C)c(C)c2)CC1 ; *C1(O)CCN(Cc2ccc3nc[nH]c3c2)CC1 ; *C1CCN(Cc2ccc(C)cc2)CC1 ; *C1CCN(Cc2ccc(C)c(F)c2)CC1]

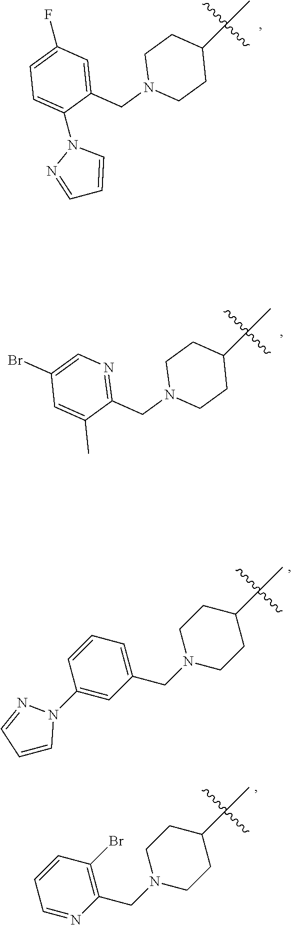 [SMILES: *C1CCN(Cc2cc(F)ccc2-n2cccn2)CC1 ; *C1CCN(Cc2ncc(Br)cc2C)CC1 ; *C1CCN(Cc2cccc(-n3cccn3)c2)CC1 ; *C1CCN(Cc2ncccc2Br)CC1]

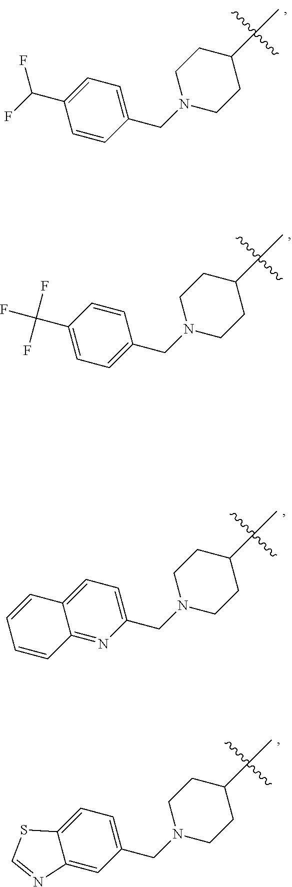 [SMILES: *C1CCN(Cc2ccc(C(F)F)cc2)CC1 ; *C1CCN(Cc2ccc(C(F)(F)F)cc2)CC1 ; *C1CCN(Cc2ccc3ccccc3n2)CC1 ; *C1CCN(Cc2ccc3scnc3c2)CC1]

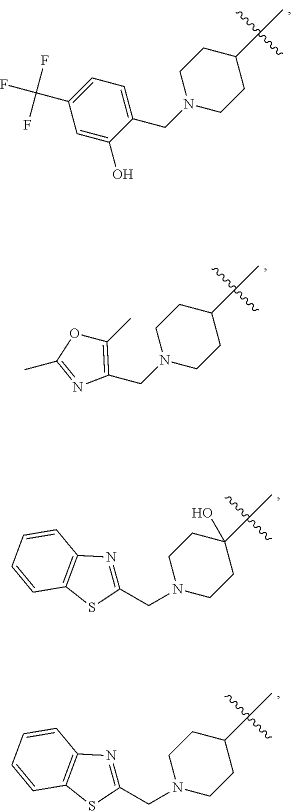 [SMILES: *C1CCN(Cc2ccc(C(F)(F)F)cc2O)CC1 ; *C1CCN(Cc2nc(C)oc2C)CC1 ; *C1(O)CCN(Cc2nc3ccccc3s2)CC1 ; *C1CCN(Cc2nc3ccccc3s2)CC1]

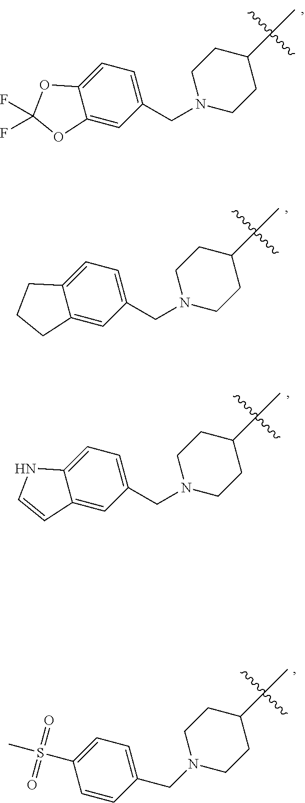 [SMILES: *C1CCN(Cc2ccc3c(c2)OC(F)(F)O3)CC1 ; *C1CCN(Cc2ccc3c(c2)CCC3)CC1 ; *C1CCN(Cc2ccc3[nH]ccc3c2)CC1 ; *C1CCN(Cc2ccc(S(C)(=O)=O)cc2)CC1]

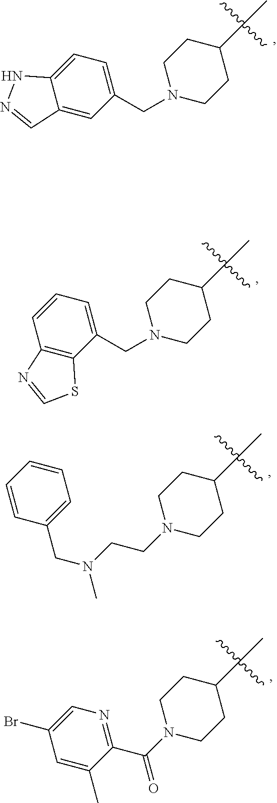 [SMILES: *C1CCN(Cc2ccc3[nH]ncc3c2)CC1 ; *C1CCN(Cc2cccc3ncsc23)CC1 ; *C1CCN(CCN(C)Cc2ccccc2)CC1 ; *C1CCN(C(=O)c2ncc(Br)cc2C)CC1]

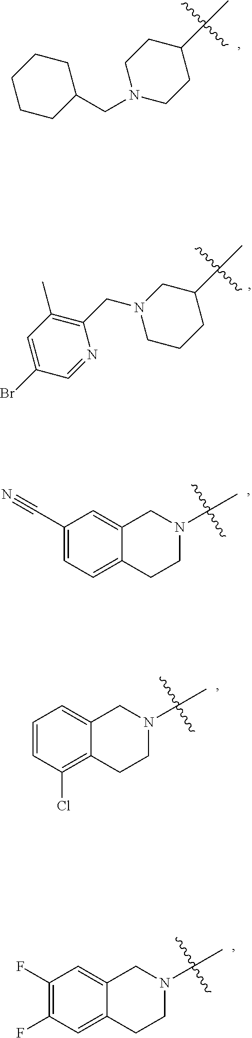 [SMILES: *C1CCN(CC2CCCCC2)CC1 ; *C1CCCN(Cc2ncc(Br)cc2C)C1 ; *N1CCc2ccc(C#N)cc2C1 ; *N1CCc2c(Cl)cccc2C1 ; *N1CCc2cc(F)c(F)cc2C1]

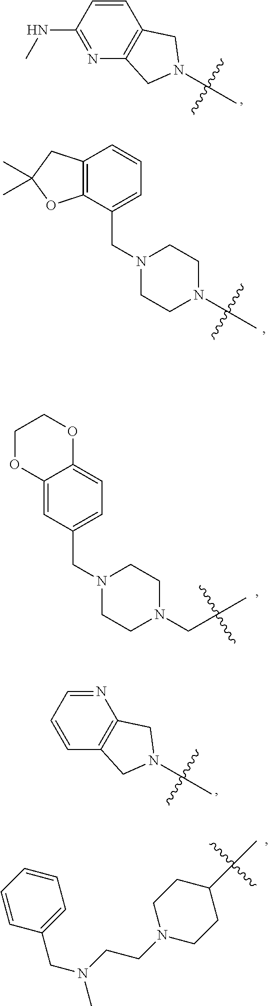 [SMILES: *N1Cc2ccc(NC)nc2C1 ; *N1CCN(Cc2cccc3c2OC(C)(C)C3)CC1 ; *CN1CCN(Cc2ccc3c(c2)OCCO3)CC1 ; *N1Cc2cccnc2C1 ; *C1CCN(CCN(C)Cc2ccccc2)CC1]

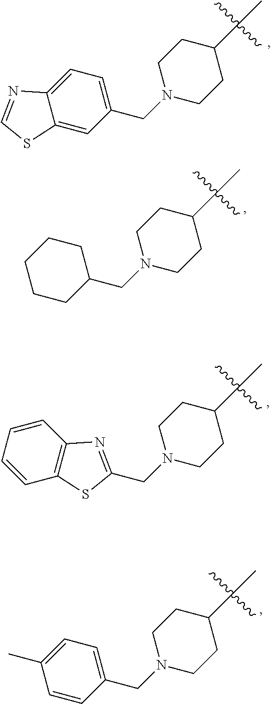 [SMILES: *C1CCN(Cc2ccc3ncsc3c2)CC1 ; *C1CCN(CC2CCCCC2)CC1 ; *C1CCN(Cc2nc3ccccc3s2)CC1 ; *C1CCN(Cc2ccc(C)cc2)CC1]

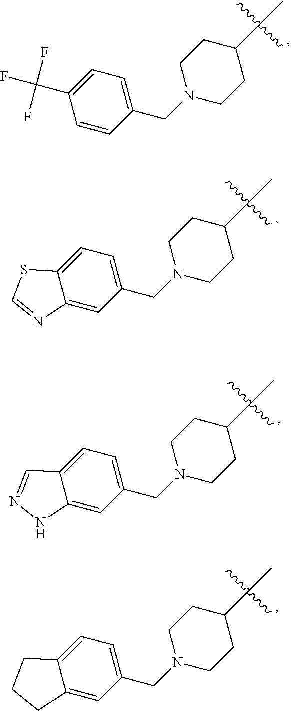 [SMILES: *C1CCN(Cc2ccc(C(F)(F)F)cc2)CC1 ; *C1CCN(Cc2ccc3scnc3c2)CC1 ; *C1CCN(Cc2ccc3cn[nH]c3c2)CC1 ; *C1CCN(Cc2ccc3c(c2)CCC3)CC1]

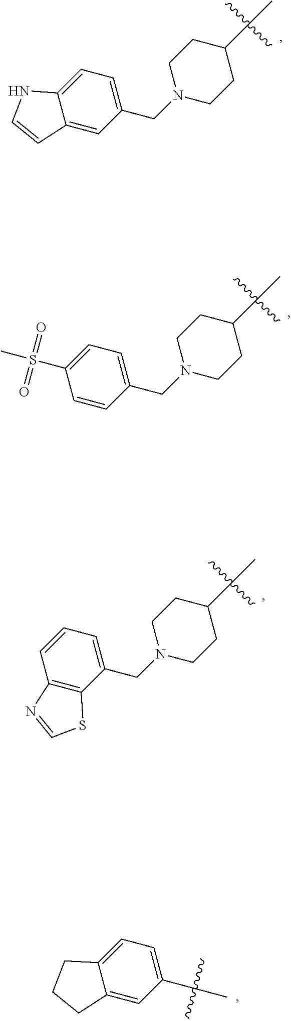 [SMILES: *C1CCN(Cc2ccc3[nH]ccc3c2)CC1 ; *C1CCN(Cc2ccc(S(C)(=O)=O)cc2)CC1 ; *C1CCN(Cc2cccc3ncsc23)CC1 ; *c1ccc2c(c1)CCC2]

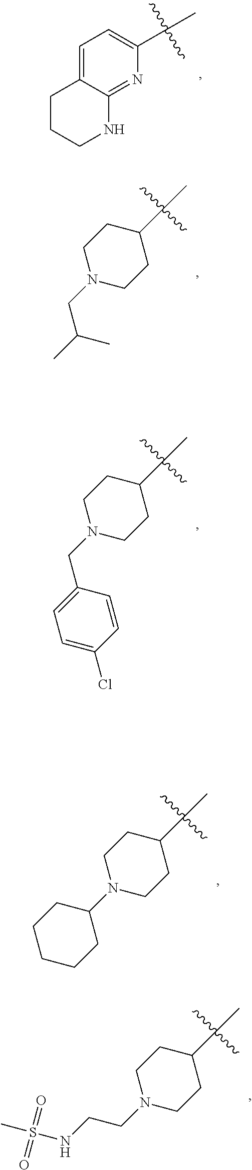 [SMILES: *c1ccc2c(n1)NCCC2 ; *C1CCN(CC(C)C)CC1 ; *C1CCN(Cc2ccc(Cl)cc2)CC1 ; *C1CCN(C2CCCCC2)CC1 ; *C1CCN(CCNS(C)(=O)=O)CC1]

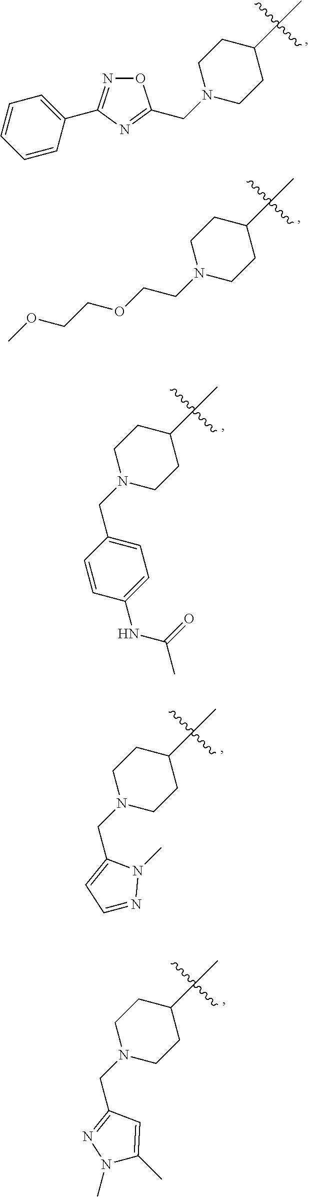 [SMILES: *C1CCN(Cc2nc(-c3ccccc3)no2)CC1 ; *C1CCN(CCOCCOC)CC1 ; *C1CCN(Cc2ccc(NC(C)=O)cc2)CC1 ; *C1CCN(Cc2ccnn2C)CC1 ; *C1CCN(Cc2cc(C)n(C)n2)CC1]

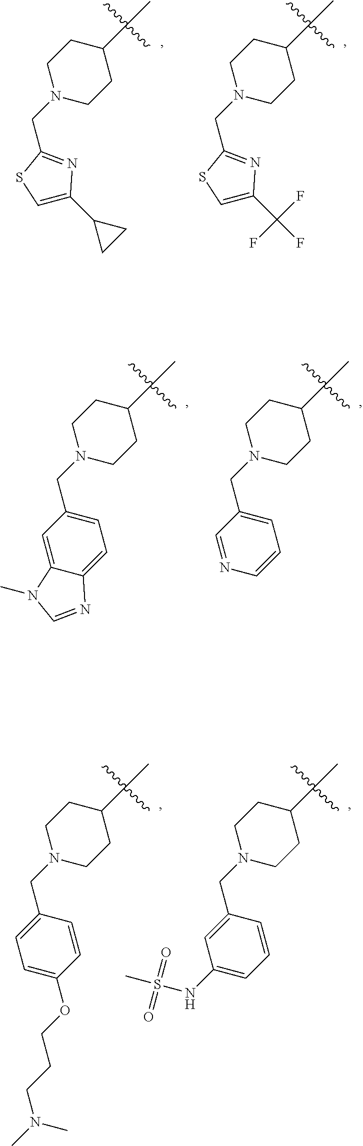 [SMILES: *C1CCN(Cc2nc(C3CC3)cs2)CC1 ; *C1CCN(Cc2nc(C(F)(F)F)cs2)CC1 ; *C1CCN(Cc2ccc3ncn(C)c3c2)CC1 ; *C1CCN(Cc2cccnc2)CC1 ; *C1CCN(Cc2ccc(OCCCN(C)C)cc2)CC1 ; *C1CCN(Cc2cccc(NS(C)(=O)=O)c2)CC1]

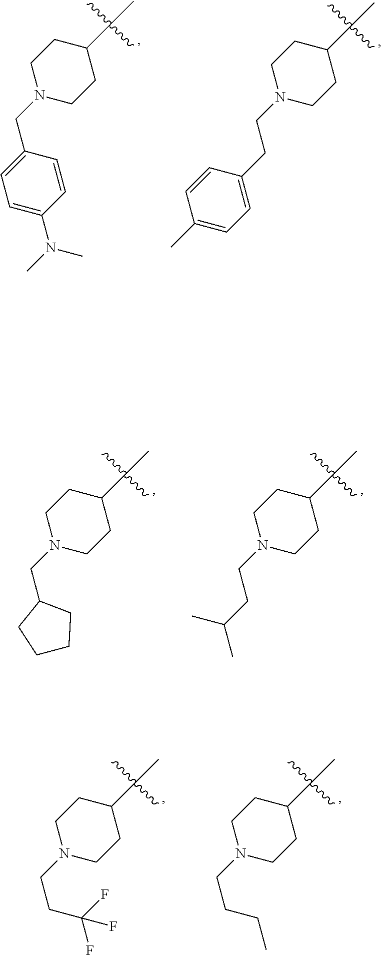 [SMILES: *C1CCN(Cc2ccc(N(C)C)cc2)CC1 ; *C1CCN(CCc2ccc(C)cc2)CC1 ; *C1CCN(CC2CCCC2)CC1 ; *C1CCN(CCC(C)C)CC1 ; *C1CCN(CCC(F)(F)F)CC1 ; *C1CCN(CCCC)CC1]

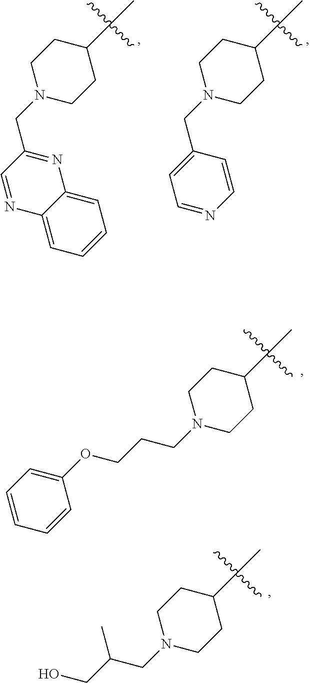 [SMILES: *C1CCN(Cc2cnc3ccccc3n2)CC1 ; *C1CCN(Cc2ccncc2)CC1 ; *C1CCN(CCCOc2ccccc2)CC1 ; *C1CCN(CC(C)CO)CC1]

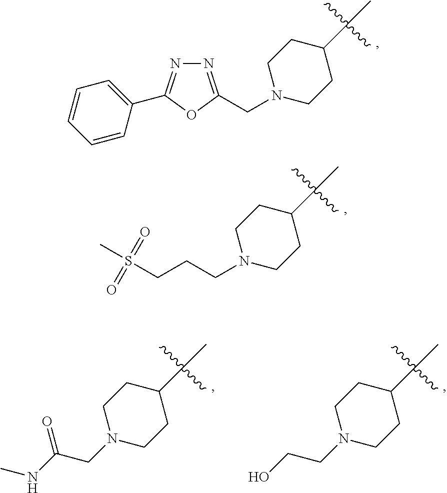 [SMILES: *C1CCN(Cc2nnc(-c3ccccc3)o2)CC1 ; *C1CCN(CCCS(C)(=O)=O)CC1 ; *C1CCN(CC(=O)NC)CC1 ; *C1CCN(CCO)CC1]

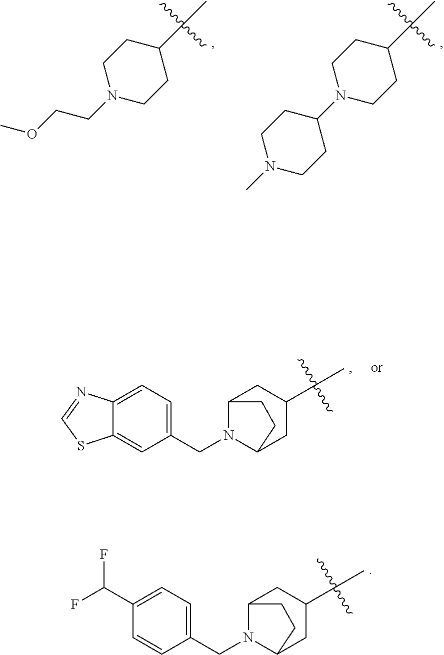 [SMILES: *C1CCN(CCOC)CC1 ; *C1CCN(C2CCN(C)CC2)CC1 ; *C1CC2CCC(C1)N2Cc1ccc2ncsc2c1 ; *C1CC2CCC(C1)N2Cc1ccc(C(F)F)cc1]

In some embodiments, the

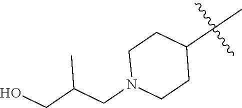 [SMILES: *C1CCN(CC(C)CO)CC1]

moiety of Ring A is

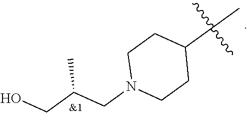 [SMILES: *C1CCN(C[C@@H](C)CO)CC1]

In some embodiments, the

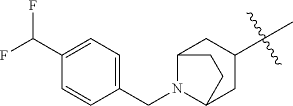 [SMILES: *C1CC2CCC(C1)N2Cc1ccc(C(F)F)cc1]

moiety of Ring A is

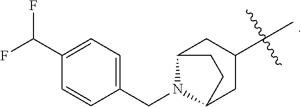 [SMILES: *C1C[C@H]2CC[C@@H](C1)N2Cc1ccc(C(F)F)cc1]

In some embodiments, Ring A is optionally substituted 5-9 membered heteroaryl that contains 1-3 heteroatoms selected from nitrogen, oxygen, and sulfur. In some embodiments, Ring A is 5-9 membered heteroaryl that contains 1-3 heteroatoms selected from nitrogen, oxygen, and sulfur, optionally substituted with optionally substituted C₁-C₃ alkyl, optionally substituted amine, or C₄-C₆ cycloalkyl. In some embodiments, Ring A is 5-9 membered heteroaryl that contains 1-3 heteroatoms selected from nitrogen, oxygen, and sulfur, optionally substituted with C₁-C₃ alkyl, amine, or C₄ cycloalkyl. In some embodiments, Ring A is 5-9 membered heteroaryl that contains 1-3 heteroatoms selected from nitrogen, oxygen, and sulfur, optionally substituted with C₁ alkyl, amine, or C₄ cycloalkyl. In some embodiments, Ring A is 5-9 membered heteroaryl that contains 1-3 heteroatoms selected from nitrogen, oxygen, and sulfur, optionally substituted with —CH₃, amine, or cyclobutyl. In some embodiments, Ring A is

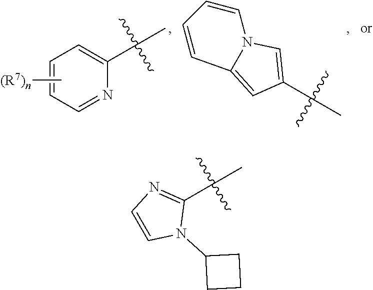

wherein R⁷ is optionally substituted C₁-C₃ alkyl or optionally substituted amine and n is 0, 1, 2, 3, or 4. In some embodiments, the

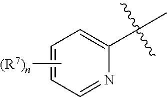

moiety of Ring A is

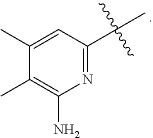 [SMILES: *c1cc(C)c(C)c(N)n1]

In some embodiments, the compound of Formula (I) is a compound of Formula (IIa), (IIb), (IIc), or (IId):

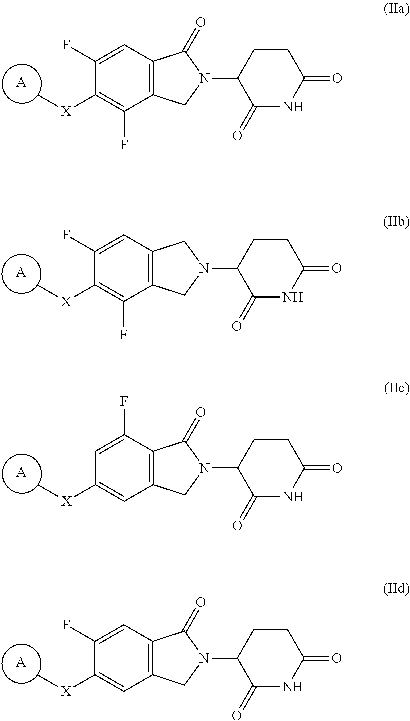

wherein X and Ring A are as as described for Formula (I).

In some embodiments, the compound of Formula (I) is a compound of Formula (IIIa), (IIIb), (IIIc), or (IIId):

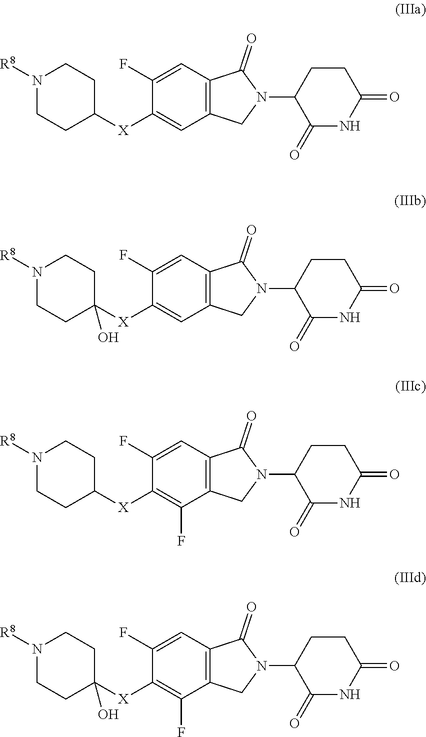

wherein R⁸ is optionally substituted C₁-C₆ alkyl or optionally substituted C₃-C₆ cycloalkyl.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of R², X, Ring A, R³, R⁴, R⁵, R⁶, R⁷, and R⁸ the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (IIa), (IIb), (IIc), (IId), (IIIa), (IIIb), (IIIc), and (IIId), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | | |
|---|---|---|
| No. | Structure | Name |
| 1 | 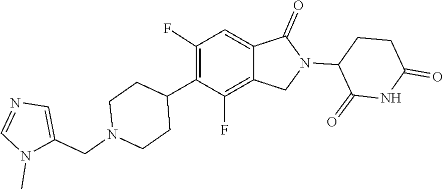 [SMILES: Cn1cncc1CN1CCC(c2c(F)cc3c(c2F)CN(C2CCC(=O)NC2=O)C3=O)CC1] | 3-(4,6-difluoro-5-(1-((1- methyl-1H-imidazol-5- yl)methyl)piperidin-4-yl)-1- oxoisoindolin-2- yl)piperidine-2,6-dione |
| 2 | 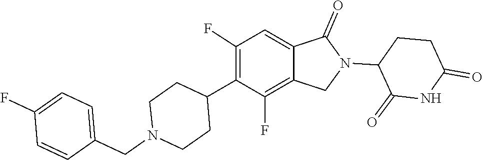 [SMILES: O=C1CCC(N2Cc3c(cc(F)c(C4CCN(Cc5ccc(F)cc5)CC4)c3F)C2=O)C(=O)N1] | 3-(4,6-difluoro-5-(1-(4- fluorobenzyl)piperidin-4-yl)- 1-oxoisoindolin-2- yl)piperidine-2,6-dione |
| 3 | 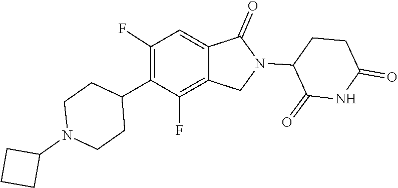 [SMILES: O=C1CCC(N2Cc3c(cc(F)c(C4CCN(C5CCC5)CC4)c3F)C2=O)C(=O)N1] | 3-(5-(1-cyclobutylpiperidin- 4-yl)-4,6-difluoro-1- oxoisoindolin-2- yl)piperidine-2,6-dione |
| 4 | 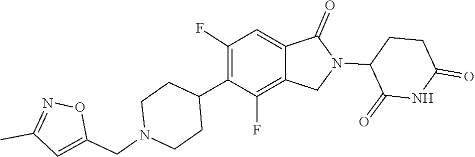 [SMILES: Cc1cc(CN2CCC(c3c(F)cc4c(c3F)CN(C3CCC(=O)NC3=O)C4=O)CC2)on1] | 3-(4,6-difluoro-5-(1-((3- methylisoxazol-5- yl)methyl)piperidin-4-yl)-1- oxoisoindolin-2- yl)piperidine-2,6-dione |
| 5 | 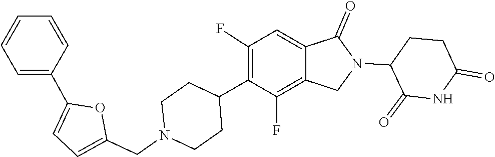 [SMILES: O=C1CCC(N2Cc3c(cc(F)c(C4CCN(Cc5ccc(-c6ccccc6)o5)CC4)c3F)C2=O)C(=O)N1] | 3-(4,6-difluoro-1-oxo-5-(1- ((5-phenylfuran-2- yl)methyl)piperidin-4- yl)isoindolin-2-yl)piperidine- 2,6-dione |
| 6 | 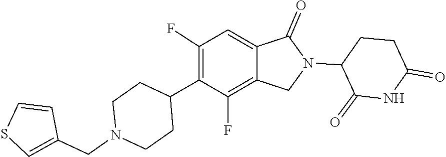 [SMILES: O=C1CCC(N2Cc3c(cc(F)c(C4CCN(Cc5ccsc5)CC4)c3F)C2=O)C(=O)N1] | 3-(4,6-difluoro-1-oxo-5-(1- (thiophen-3- ylmethyl)piperidin-4- yl)isoindolin-2-yl)piperidine- 2,6-dione |
| 7 | 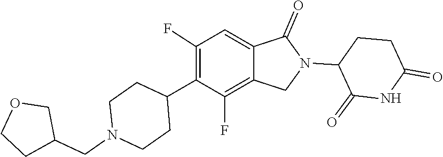 [SMILES: O=C1CCC(N2Cc3c(cc(F)c(C4CCN(CC5CCOC5)CC4)c3F)C2=O)C(=O)N1] | 3-(4,6-difluoro-1-oxo-5-(1- ((tetrahydrofuran-3- yl)methyl)piperidin-4- yl)isoindolin-2-yl)piperidine- 2,6-dione |
| 8 | 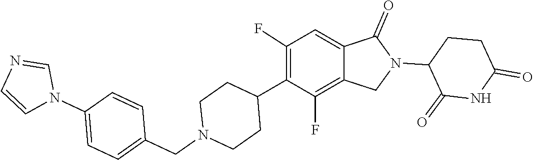 [SMILES: O=C1CCC(N2Cc3c(cc(F)c(C4CCN(Cc5ccc(-n6ccnc6)cc5)CC4)c3F)C2=O)C(=O)N1] | 3-(5-(1-(4-(1H-imidazol-1- yl)benzyl)piperidin-4-yl)- 4,6-difluoro-1-oxoisoindolin- 2-yl)piperidine-2,6-dione |
| 9 | 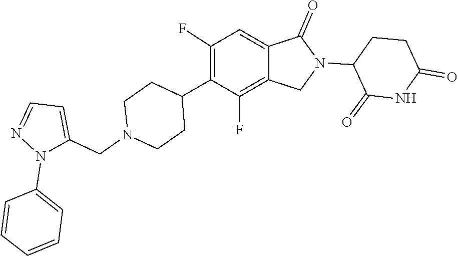 [SMILES: O=C1CCC(N2Cc3c(cc(F)c(C4CCN(Cc5ccnn5-c5ccccc5)CC4)c3F)C2=O)C(=O)N1] | 3-(4,6-difluoro-1-oxo-5-(1- ((1-phenyl-1H-pyrazol-5- yl)methyl)piperidin-4- yl)isoindolin-2-yl)piperidine- 2,6-dione |
| 10 | 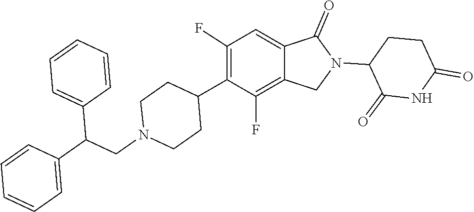 [SMILES: O=C1CCC(N2Cc3c(cc(F)c(C4CCN(CC(c5ccccc5)c5ccccc5)CC4)c3F)C2=O)C(=O)N1] | 3-(5-(1-(2,2- diphenylethyl)piperidin-4- yl)-4,6-difluoro-1- oxoisoindolin-2- yl)piperidine-2,6-dione |
| 11 | 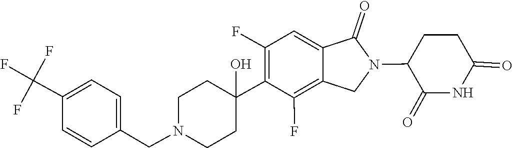 [SMILES: O=C1CCC(N2Cc3c(cc(F)c(C4(O)CCN(Cc5ccc(C(F)(F)F)cc5)CC4)c3F)C2=O)C(=O)N1] | 3-(4,6-difluoro-5-(4- hydroxy-1-(4- (trifluoromethyl)benzyl)piper idin-4-yl)-1-oxoisoindolin-2- yl)piperidine-2,6-dione |
| 12 | 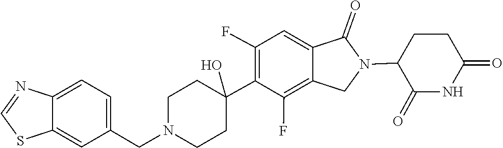 [SMILES: O=C1CCC(N2Cc3c(cc(F)c(C4(O)CCN(Cc5ccc6ncsc6c5)CC4)c3F)C2=O)C(=O)N1] | 3-(5-(1-(benzo[d]thiazol-6- ylmethyl)-4- hydroxypiperidin-4-yl)-4,6- difluoro-1-oxoisoindolin-2- yl)piperidine-2,6-dione |
| 13 | 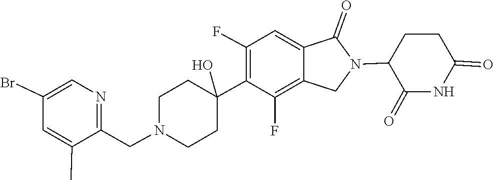 [SMILES: Cc1cc(Br)cnc1CN1CCC(O)(c2c(F)cc3c(c2F)CN(C2CCC(=O)NC2=O)C3=O)CC1] | 3-(5-(1-((5-bromo-3- methylpyridin-2-yl)methyl)- 4-hydroxypiperidin-4-yl)- 4,6-difluoro-1-oxoisoindolin- 2-yl)piperidine-2,6-dione |
| 14 | 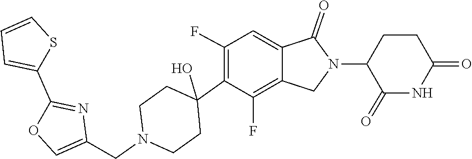 [SMILES: O=C1CCC(N2Cc3c(cc(F)c(C4(O)CCN(Cc5coc(-c6cccs6)n5)CC4)c3F)C2=O)C(=O)N1] | 3-(4,6-difluoro-5-(4- hydroxy-1-((2-(thiophen-2- yl)oxazol-4- yl)methyl)piperidin-4-yl)-1- oxoisoindolin-2- yl)piperidine-2,6-dione |
| 15 | 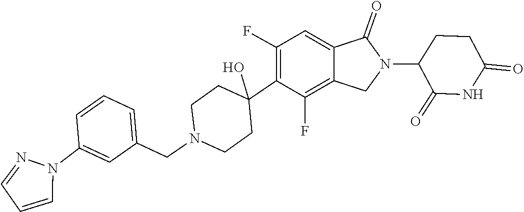 [SMILES: O=C1CCC(N2Cc3c(cc(F)c(C4(O)CCN(Cc5cccc(-n6cccn6)c5)CC4)c3F)C2=O)C(=O)N1] | 3-(5-(1-(3-(1H-pyrazol-1- yl)benzyl)-4- hydroxypiperidin-4-yl)-4,6- difluoro-1-oxoisoindolin-2- yl)piperidine-2,6-dione |
| 16 | 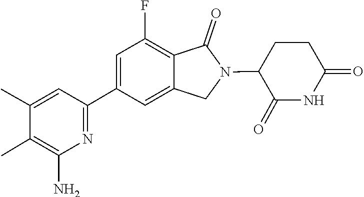 [SMILES: Cc1cc(-c2cc(F)c3c(c2)CN(C2CCC(=O)NC2=O)C3=O)nc(N)c1C] | 3-(5-(6-amino-4,5- dimethylpyridin-2-yl)-7- fluoro-1-oxoisoindolin-2- yl)piperidine-2,6-dione |
| 17 | 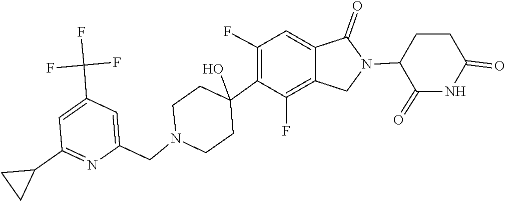 [SMILES: O=C1CCC(N2Cc3c(cc(F)c(C4(O)CCN(Cc5cc(C(F)(F)F)cc(C6CC6)n5)CC4)c3F)C2=O)C(=O)N1] | 3-(5-(1-((6-cyclopropyl-4- (trifluoromethyl)pyridin-2- yl)methyl)-4- hydroxypiperidin-4-yl)-4,6- difluoro-1-oxoisoindolin-2- yl)piperidine-2,6-dione |
| 18 | 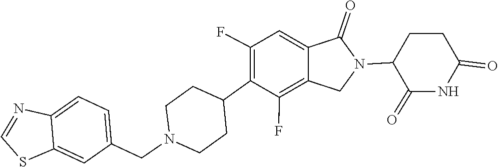 [SMILES: O=C1CCC(N2Cc3c(cc(F)c(C4CCN(Cc5ccc6ncsc6c5)CC4)c3F)C2=O)C(=O)N1] | 3-(5-(1-(benzo[d]thiazol-6- ylmethyl)piperidin-4-yl)-4,6- difluoro-1-oxoisoindolin-2- yl)piperidine-2,6-dione |
| 19 | 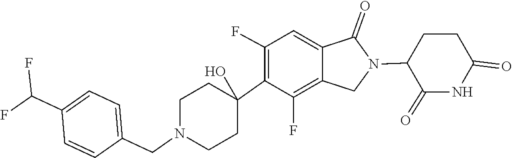 [SMILES: O=C1CCC(N2Cc3c(cc(F)c(C4(O)CCN(Cc5ccc(C(F)F)cc5)CC4)c3F)C2=O)C(=O)N1] | 3-(5-(1-(4- (difluoromethyl)benzyl)-4- hydroxypiperidin-4-yl)-4,6- difluoro-1-oxoisoindolin-2- yl)piperidine-2,6-dione |
| 20 | 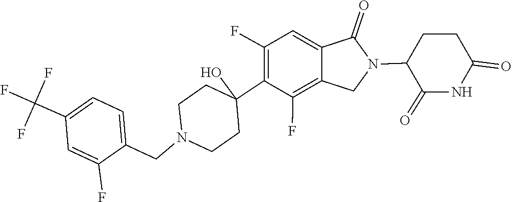 [SMILES: O=C1CCC(N2Cc3c(cc(F)c(C4(O)CCN(Cc5ccc(C(F)(F)F)cc5F)CC4)c3F)C2=O)C(=O)N1] | 3-(4,6-difluoro-5-(1-(2- fluoro-4- (trifluoromethyl)benzyl)-4- hydroxypiperidin-4-yl)-1- oxoisoindolin-2- yl)piperidine-2,6-dione |
| 21 | 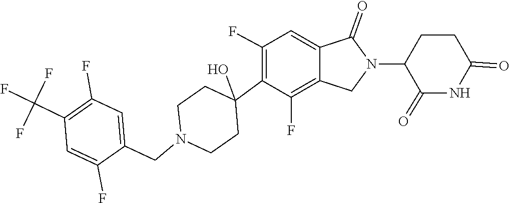 [SMILES: O=C1CCC(N2Cc3c(cc(F)c(C4(O)CCN(Cc5cc(F)c(C(F)(F)F)cc5F)CC4)c3F)C2=O)C(=O)N1] | 3-(5-(1-(2,5-difluoro-4- (trifluoromethyl)benzyl)-4- hydroxypiperidin-4-yl)-4,6- difluoro-1-oxoisoindolin-2- yl)piperidine-2,6-dione |
| 22 | 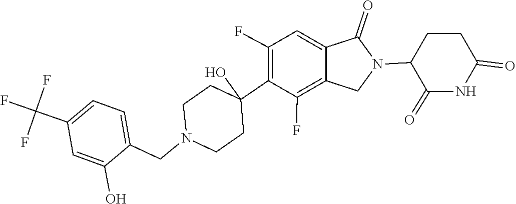 [SMILES: O=C1CCC(N2Cc3c(cc(F)c(C4(O)CCN(Cc5ccc(C(F)(F)F)cc5O)CC4)c3F)C2=O)C(=O)N1] | 3-(4,6-difluoro-5-(4- hydroxy-1-(2-hydroxy-4- (trifluoromethyl)benzyl)piper idin-4-yl)-1-oxoisoindolin-2- yl)piperidine-2,6-dione |
| 23 | 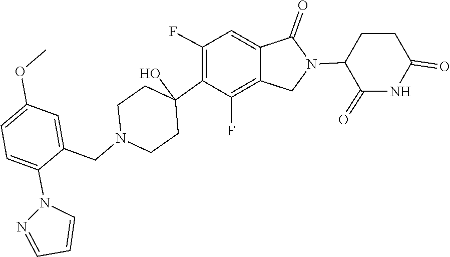 [SMILES: COc1ccc(-n2cccn2)c(CN2CCC(O)(c3c(F)cc4c(c3F)CN(C3CCC(=O)NC3=O)C4=O)CC2)c1] | 3-(4,6-difluoro-5-(4- hydroxy-1-(5-methoxy-2- (1H-pyrazol-1- yl)benzyl)piperidin-4-yl)-1- oxoisoindolin-2- yl)piperidine-2,6-dione |
| 24 | 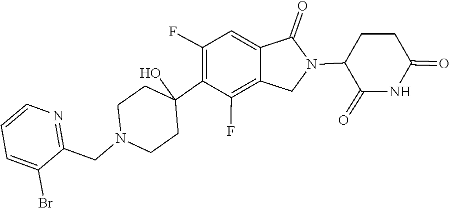 [SMILES: O=C1CCC(N2Cc3c(cc(F)c(C4(O)CCN(Cc5ncccc5Br)CC4)c3F)C2=O)C(=O)N1] | 3-(5-(1-((3-bromopyridin-2- yl)methyl)-4- hydroxypiperidin-4-yl)-4,6- difluoro-1-oxoisoindolin-2- yl)piperidine-2,6-dione |
| 25 | 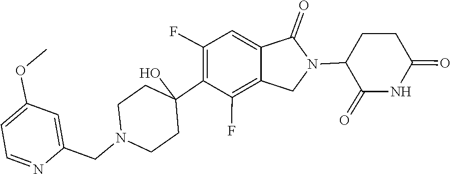 [SMILES: COc1ccnc(CN2CCC(O)(c3c(F)cc4c(c3F)CN(C3CCC(=O)NC3=O)C4=O)CC2)c1] | 3-(4,6-difluoro-5-(4- hydroxy-1-((4- methoxypyridin-2- yl)methyl)piperidin-4-yl)-1- oxoisoindolin-2- yl)piperidine-2,6-dione |
| 26 | 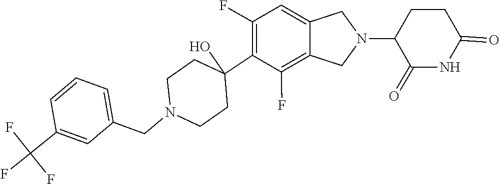 [SMILES: O=C1CCC(N2Cc3cc(F)c(C4(O)CCN(Cc5cccc(C(F)(F)F)c5)CC4)c(F)c3C2)C(=O)N1] | 3-(4,6-difluoro-5-(4- hydroxy-1-(3- (trifluoromethyl)benzyl)piper idin-4-yl)-1-oxoisoindolin-2- yl)piperidine-2,6-dione |
| 27 | 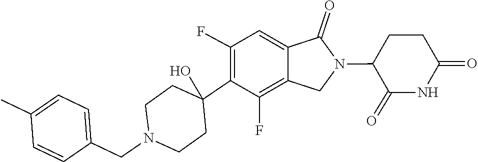 [SMILES: Cc1ccc(CN2CCC(O)(c3c(F)cc4c(c3F)CN(C3CCC(=O)NC3=O)C4=O)CC2)cc1] | 3-(4,6-difluoro-5-(4- hydroxy-1-(4- methylbenzyl)piperidin-4- yl)-1-oxoisoindolin-2- yl)piperidine-2,6-dione |
| 28 | 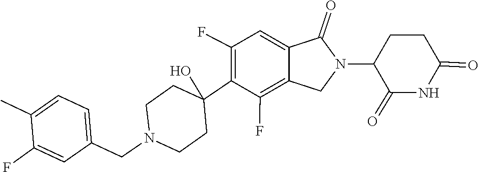 [SMILES: Cc1ccc(CN2CCC(O)(c3c(F)cc4c(c3F)CN(C3CCC(=O)NC3=O)C4=O)CC2)cc1F] | 3-(4,6-difluoro-5-(1-(3- fluoro-4-methylbenzyl)-4- hydroxypiperidin-4-yl)-1- oxoisoindolin-2- yl)piperidine-2,6-dione |
| 29 | 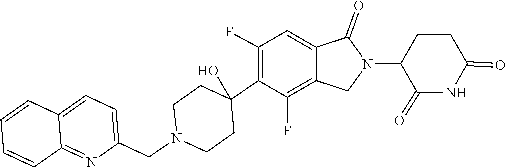 [SMILES: O=C1CCC(N2Cc3c(cc(F)c(C4(O)CCN(Cc5ccc6ccccc6n5)CC4)c3F)C2=O)C(=O)N1] | 3-(4,6-difluoro-5-(4- hydroxy-1-(quinolin-2- ylmethyl)piperidin-4-yl)-1- oxoisoindolin-2- yl)piperidine-2,6-dione |
| 30 | 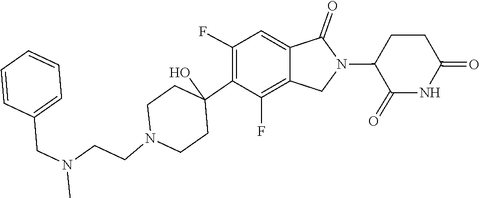 [SMILES: CN(CCN1CCC(O)(c2c(F)cc3c(c2F)CN(C2CCC(=O)NC2=O)C3=O)CC1)Cc1ccccc1] | 3-(5-(1-(2- (benzyl(methyl)amino)ethyl) -4-hydroxypiperidin-4-yl)- 4,6-difluoro-1-oxoisoindolin- 2-yl)piperidine-2,6-dione |
| 31 | 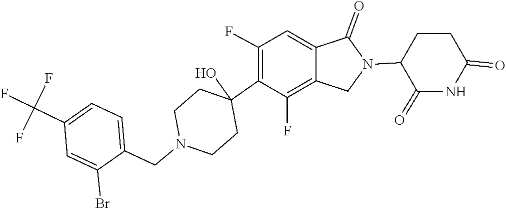 [SMILES: O=C1CCC(N2Cc3c(cc(F)c(C4(O)CCN(Cc5ccc(C(F)(F)F)cc5Br)CC4)c3F)C2=O)C(=O)N1] | 3-(5-(1-(2-bromo-4- (trifluoromethyl)benzyl)-4- hydroxypiperidin-4-yl)-4,6- difluoro-1-oxoisoindolin-2- yl)piperidine-2,6-dione |
| 32 | 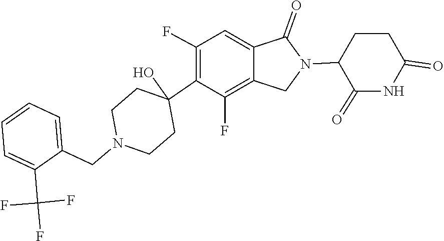 [SMILES: O=C1CCC(N2Cc3c(cc(F)c(C4(O)CCN(Cc5ccccc5C(F)(F)F)CC4)c3F)C2=O)C(=O)N1] | 3-(4,6-difluoro-5-(4- hydroxy-1-(2- (trifluoromethyl)benzyl)piper idin-4-yl)-1-oxoisoindolin-2- yl)piperidine-2,6-dione |
| 33 | 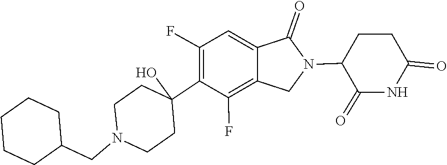 [SMILES: O=C1CCC(N2Cc3c(cc(F)c(C4(O)CCN(CC5CCCCC5)CC4)c3F)C2=O)C(=O)N1] | 3-(5-(1-(cyclohexylmethyl)- 4-hydroxypiperidin-4-yl)- 4,6-difluoro-1-oxoisoindolin- 2-yl)piperidine-2,6-dione |
| 34 | 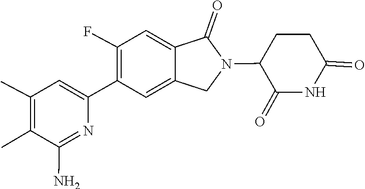 [SMILES: Cc1cc(-c2cc3c(cc2F)C(=O)N(C2CCC(=O)NC2=O)C3)nc(N)c1C] | 3-(5-(6-amino-4,5- dimethylpyridin-2-yl)-6- fluoro-1-oxoisoindolin-2- yl)piperidine-2,6-dione |
| 35 | 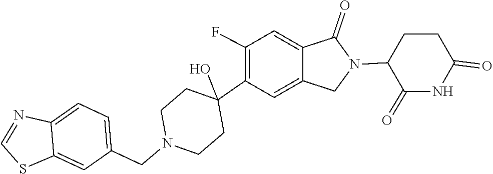 [SMILES: O=C1CCC(N2Cc3cc(C4(O)CCN(Cc5ccc6ncsc6c5)CC4)c(F)cc3C2=O)C(=O)N1] | 3-(5-(1-(benzo[d]thiazol-6- ylmethyl)-4- hydroxypiperidin-4-yl)-6- fluoro-1-oxoisoindolin-2- yl)piperidine-2,6-dione |
| 36 | 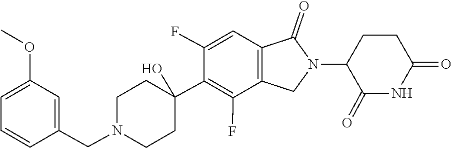 [SMILES: COc1cccc(CN2CCC(O)(c3c(F)cc4c(c3F)CN(C3CCC(=O)NC3=O)C4=O)CC2)c1] | 3-(4,6-difluoro-5-(4- hydroxy-1-(3- methoxybenzyl)piperidin-4- yl)-1-oxoisoindolin-2- yl)piperidine-2,6-dione |
| 37 | 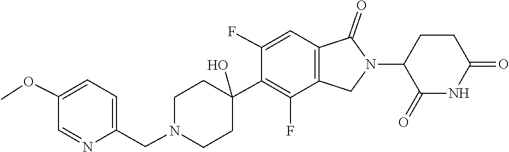 [SMILES: COc1ccc(CN2CCC(O)(c3c(F)cc4c(c3F)CN(C3CCC(=O)NC3=O)C4=O)CC2)nc1] | 3-(4,6-difluoro-5-(4- hydroxy-1-((5- methoxypyridin-2- yl)methyl)piperidin-4-yl)-1- oxoisoindolin-2- yl)piperidine-2,6-dione |
| 38 | 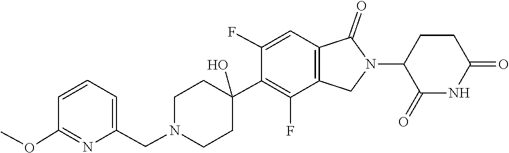 [SMILES: COc1cccc(CN2CCC(O)(c3c(F)cc4c(c3F)CN(C3CCC(=O)NC3=O)C4=O)CC2)n1] | 3-(4,6-difluoro-5-(4- hydroxy-1-((6- methoxypyridin-2- yl)methyl)piperidin-4-yl)-1- oxoisoindolin-2- yl)piperidine-2,6-dione |
| 39 | 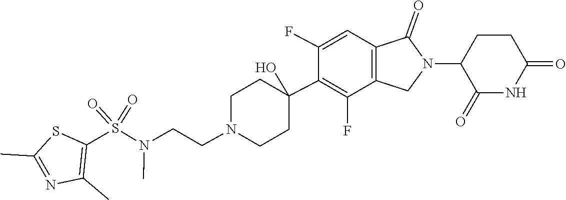 [SMILES: Cc1nc(C)c(S(=O)(=O)N(C)CCN2CCC(O)(c3c(F)cc4c(c3F)CN(C3CCC(=O)NC3=O)C4=O)CC2)s1] | N-(2-(4-(2-(2,6- dioxopiperidin-3-yl)-4,6- difluoro-1-oxoisoindolin-5- yl)-4-hydroxypiperidin-1- yl)ethyl)-N,2,4- trimethylthiazole-5- sulfonamide |
| 40 | 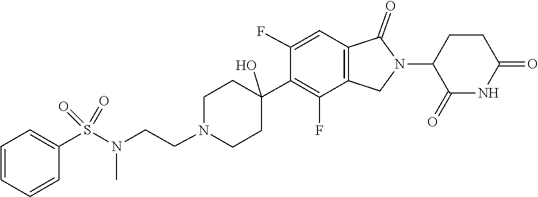 [SMILES: CN(CCN1CCC(O)(c2c(F)cc3c(c2F)CN(C2CCC(=O)NC2=O)C3=O)CC1)S(=O)(=O)c1ccccc1] | N-(2-(4-(2-(2,6- dioxopiperidin-3-yl)-4,6- difluoro-1-oxoisoindolin-5- yl)-4-hydroxypiperidin-1- yl)ethyl)-N- methylbenzenesulfonamide |
| 41 | 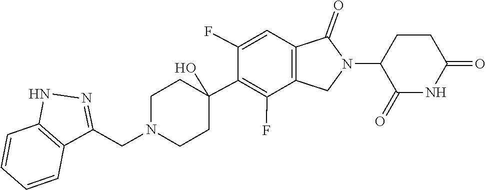 [SMILES: O=C1CCC(N2Cc3c(cc(F)c(C4(O)CCN(Cc5n[nH]c6ccccc56)CC4)c3F)C2=O)C(=O)N1] | 3-(5-(1-((1H-indazol-3- yl)methyl)-4- hydroxypiperidin-4-yl)-4,6- difluoro-1-oxoisoindolin-2- yl)piperidine-2,6-dione |
| 42 | 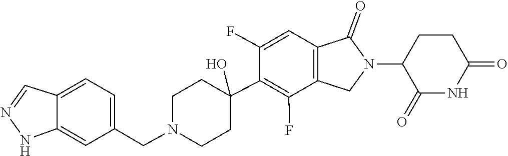 | 3-(5-(1-((1H-indazol-6- yl)methyl)-4- hydroxypiperidin-4-yl)-4,6- difluoro-1-oxoisoindolin-2- yl)piperidine-2,6-dione |
| 43 | 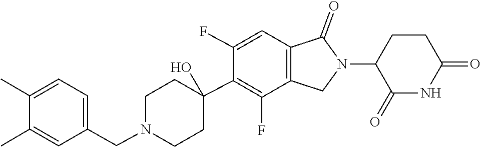 [SMILES: Cc1ccc(CN2CCC(O)(c3c(F)cc4c(c3F)CN(C3CCC(=O)NC3=O)C4=O)CC2)cc1C] | 3-(5-(1-(3,4- dimethylbenzyl)-4- hydroxypiperidin-4-yl)-4,6- difluoro-1-oxoisoindolin-2- yl)piperidine-2,6-dione |
| 44 | 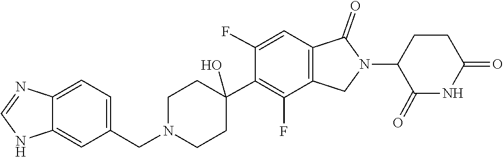 [SMILES: O=C1CCC(N2Cc3c(cc(F)c(C4(O)CCN(Cc5ccc6nc[nH]c6c5)CC4)c3F)C2=O)C(=O)N1] | 3-(5-(1-((1H- benzo[d]imidazol-6- yl)methyl)-4- hydroxypiperidin-4-yl)-4,6- difluoro-1-oxoisoindolin-2- yl)piperidine-2,6-dione |
| 45 | 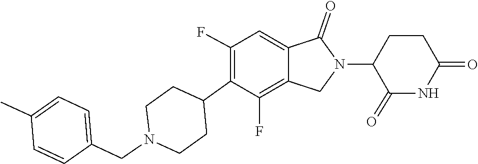 [SMILES: Cc1ccc(CN2CCC(c3c(F)cc4c(c3F)CN(C3CCC(=O)NC3=O)C4=O)CC2)cc1] | 3-(4,6-difluoro-5-(1-(4- methylbenzyl)piperidin-4- yl)-1-oxoisoindolin-2- yl)piperidine-2,6-dione |
| 46 | 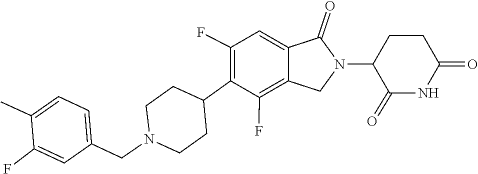 [SMILES: Cc1ccc(CN2CCC(c3c(F)cc4c(c3F)CN(C3CCC(=O)NC3=O)C4=O)CC2)cc1F] | 3-(4,6-difluoro-5-(1-(3- fluoro-4- methylbenzyl)piperidin-4- yl)-1-oxoisoindolin-2- yl)piperidine-2,6-dione |
| 47 | 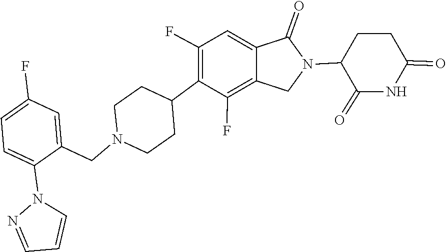 [SMILES: O=C1CCC(N2Cc3c(cc(F)c(C4CCN(Cc5cc(F)ccc5-n5cccn5)CC4)c3F)C2=O)C(=O)N1] | 3-(4,6-difluoro-5-(1-(5- fluoro-2-(1H-pyrazol-1- yl)benzyl)piperidin-4-yl)-1- oxoisoindolin-2- yl)piperidine-2,6-dione |
| 48 | 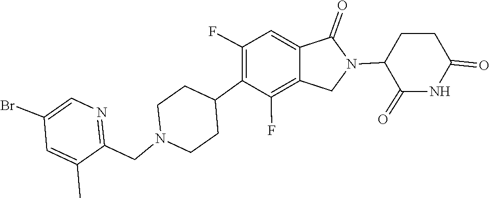 [SMILES: Cc1cc(Br)cnc1CN1CCC(c2c(F)cc3c(c2F)CN(C2CCC(=O)NC2=O)C3=O)CC1] | 3-(5-(1-((5-bromo-3- methylpyridin-2- yl)methyl)piperidin-4-yl)- 4,6-difluoro-1-oxoisoindolin- 2-yl)piperidine-2,6-dione |
| 49 | 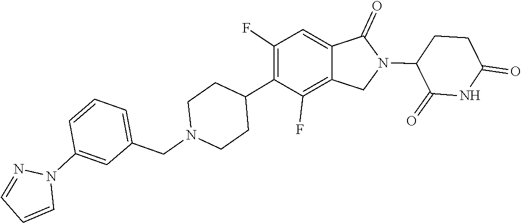 [SMILES: O=C1CCC(N2Cc3c(cc(F)c(C4CCN(Cc5cccc(-n6cccn6)c5)CC4)c3F)C2=O)C(=O)N1] | 3-(5-(1-(3-(1H-pyrazol-1- yl)benzyl)piperidin-4-yl)- 4,6-difluoro-1-oxoisoindolin- 2-yl)piperidine-2,6-dione |
| 50 | 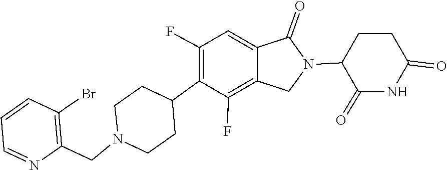 [SMILES: O=C1CCC(N2Cc3c(cc(F)c(C4CCN(Cc5ncccc5Br)CC4)c3F)C2=O)C(=O)N1] | 3-(5-(1-((3-bromopyridin-2- yl)methyl)piperidin-4-yl)- 4,6-difluoro-1-oxoisoindolin- 2-yl)piperidine-2,6-dione |
| 51 | 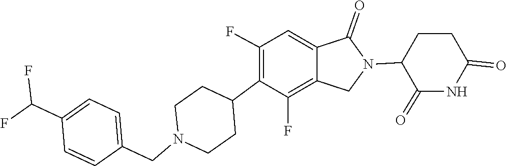 [SMILES: O=C1CCC(N2Cc3c(cc(F)c(C4CCN(Cc5ccc(C(F)F)cc5)CC4)c3F)C2=O)C(=O)N1] | 3-(5-(1-(4- (difluoromethyl)benzyl) piperidin-4-yl)-4,6-difluoro-1- oxoisoindolin-2- yl)piperidine-2,6-dione |
| 52 | 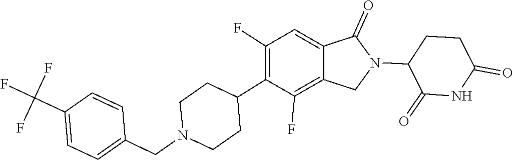 [SMILES: O=C1CCC(N2Cc3c(cc(F)c(C4CCN(Cc5ccc(C(F)(F)F)cc5)CC4)c3F)C2=O)C(=O)N1] | 3-(4,6-difluoro-1-oxo-5-(1- (4- (trifluoromethyl)benzyl) piperidin-4-yl)isoindolin-2- yl)piperidine-2,6-dione |
| 53 | 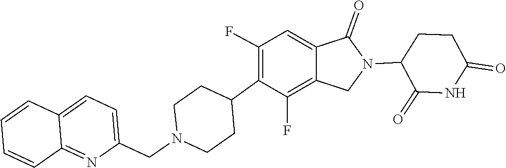 [SMILES: O=C1CCC(N2Cc3c(cc(F)c(C4CCN(Cc5ccc6ccccc6n5)CC4)c3F)C2=O)C(=O)N1] | 3-(4,6-difluoro-1-oxo-5-(1- (quinolin-2- ylmethyl)piperidin-4- yl)isoindolin-2-yl)piperidine- 2,6-dione |
| 54 | 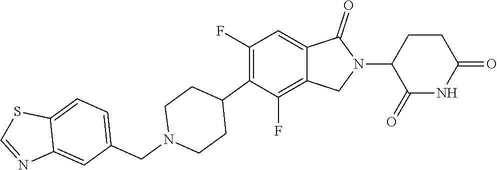 [SMILES: O=C1CCC(N2Cc3c(cc(F)c(C4CCN(Cc5ccc6scnc6c5)CC4)c3F)C2=O)C(=O)N1] | 3-(5-(1-(benzo[d]thiazol-5- ylmethyl)piperidin-4-yl)-4,6- difluoro-1-oxoisoindolin-2- yl)piperidine-2,6-dione |
| 55 | 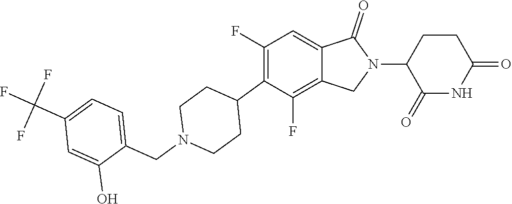 [SMILES: O=C1CCC(N2Cc3c(cc(F)c(C4CCN(Cc5ccc(C(F)(F)F)cc5O)CC4)c3F)C2=O)C(=O)N1] | 3-(4,6-difluoro-5-(1-(2- hydroxy-4-(trifluoromethyl) benzyl)piperidin- 4-yl)-1-oxoisoindolin-2- yl)piperidine-2,6-dione |
| 56 | 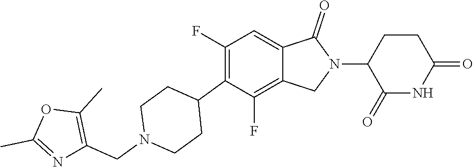 [SMILES: Cc1nc(CN2CCC(c3c(F)cc4c(c3F)CN(C3CCC(=O)NC3=O)C4=O)CC2)c(C)o1] | 3-(5-(1-((2,5- dimethyloxazol-4- yl)methyl)piperidin-4-yl)- 4,6-difluoro-1-oxoisoindolin- 2-yl)piperidine-2,6-dione |
| 57 | 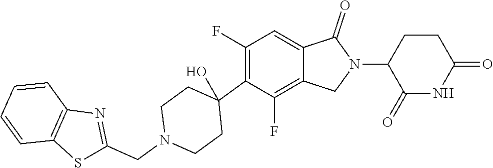 [SMILES: O=C1CCC(N2Cc3c(cc(F)c(C4(O)CCN(Cc5nc6ccccc6s5)CC4)c3F)C2=O)C(=O)N1] | 3-(5-(1-(benzo[d]thiazol-2- ylmethyl)-4- hydroxypiperidin-4-yl)-4,6- difluoro-1-oxoisoindolin-2- yl)piperidine-2,6-dione |
| 58 | 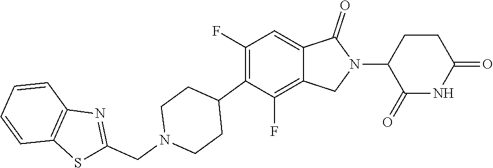 [SMILES: O=C1CCC(N2Cc3c(cc(F)c(C4CCN(Cc5nc6ccccc6s5)CC4)c3F)C2=O)C(=O)N1] | 3-(5-(1-(benzo[d]thiazol-2- ylmethyl)piperidin-4-yl)-4,6- difluoro-1-oxoisoindolin-2- yl)piperidine-2,6-dione |
| 59 | 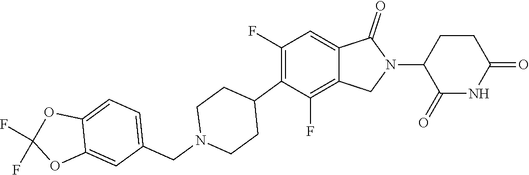 [SMILES: O=C1CCC(N2Cc3c(cc(F)c(C4CCN(Cc5ccc6c(c5)OC(F)(F)O6)CC4)c3F)C2=O)C(=O)N1] | 3-(5-(1-((2,2- difluorobenzo[d][1,3]dioxol- 5-yl)methyl)piperidin-4-yl)- 4,6-difluoro-1-oxoisoindolin- 2-yl)piperidine-2,6-dione |
| 60 | 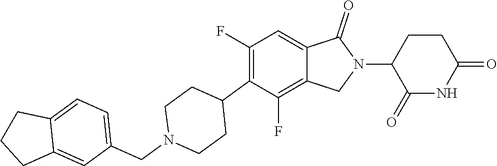 [SMILES: O=C1CCC(N2Cc3c(cc(F)c(C4CCN(Cc5ccc6c(c5)CCC6)CC4)c3F)C2=O)C(=O)N1] | 3-(5-(1-((2,3-dihydro-1H- inden-5-yl)methyl)piperidin- 4-yl)-4,6-difluoro-1- oxoisoindolin-2- yl)piperidine-2,6-dione |
| 61 | 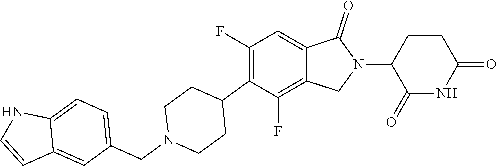 [SMILES: O=C1CCC(N2Cc3c(cc(F)c(C4CCN(Cc5ccc6[nH]ccc6c5)CC4)c3F)C2=O)C(=O)N1] | 3-(5-(1-((1H-indol-5- yl)methyl)piperidin-4-yl)- 4,6-difluoro-1-oxoisoindolin- 2-yl)piperidine-2,6-dione |
| 62 | 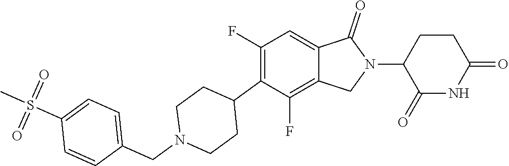 [SMILES: CS(=O)(=O)c1ccc(CN2CCC(c3c(F)cc4c(c3F)CN(C3CCC(=O)NC3=O)C4=O)CC2)cc1] | 3-(4,6-difluoro-5-(1-(4- (methylsulfonyl)benzyl)piper idin-4-yl)-1-oxoisoindolin-2- yl)piperidine-2,6-dione |
| 63 | 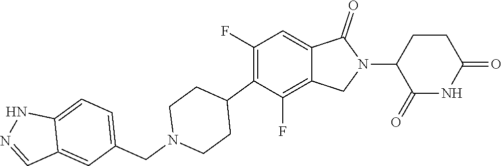 [SMILES: O=C1CCC(N2Cc3c(cc(F)c(C4CCN(Cc5ccc6[nH]ncc6c5)CC4)c3F)C2=O)C(=O)N1] | 3-(5-(1-((1H-indazol-5- yl)methyl)piperidin-4-yl)- 4,6-difluoro-1-oxoisoindolin- 2-yl)piperidine-2,6-dione |
| 64 | 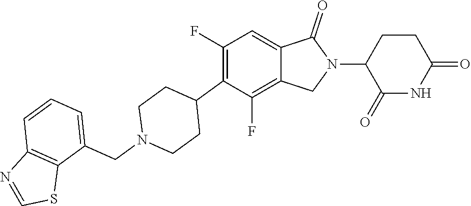 [SMILES: O=C1CCC(N2Cc3c(cc(F)c(C4CCN(Cc5cccc6ncsc56)CC4)c3F)C2=O)C(=O)N1] | 3-(5-(1-(benzo[d]thiazol-7- ylmethyl)piperidin-4-yl)-4,6- difluoro-1-oxoisoindolin-2- yl)piperidine-2,6-dione |
| 65 | 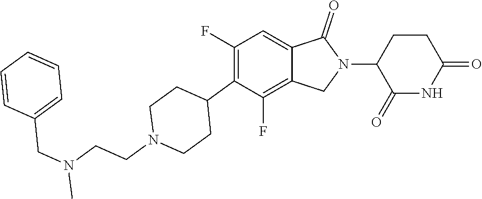 [SMILES: CN(CCN1CCC(c2c(F)cc3c(c2F)CN(C2CCC(=O)NC2=O)C3=O)CC1)Cc1ccccc1] | 3-(5-(1-(2- (benzyl(methyl)amino)ethyl) piperidin-4-yl)-4,6-difluoro- 1-oxoisoindolin-2- yl)piperidine-2,6-dione |
| 66 | 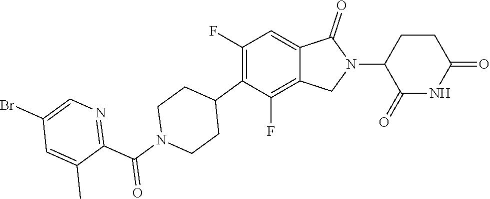 [SMILES: Cc1cc(Br)cnc1C(=O)N1CCC(c2c(F)cc3c(c2F)CN(C2CCC(=O)NC2=O)C3=O)CC1] | 3-(5-(1-(5-bromo-3- methylpicolinoyl)piperidin- 4-yl)-4,6-difluoro-1- oxoisoindolin-2- yl)piperidine-2,6-dione |
| 67 | 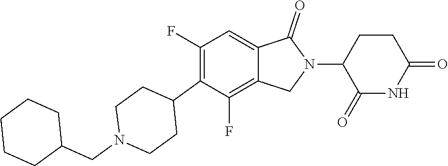 [SMILES: O=C1CCC(N2Cc3c(cc(F)c(C4CCN(CC5CCCCC5)CC4)c3F)C2=O)C(=O)N1] | 3-(5-(1- (cyclohexylmethyl)piperidin- 4-yl)-4,6-difluoro-1- oxoisoindolin-2- yl)piperidine-2,6-dione |
| 68 | 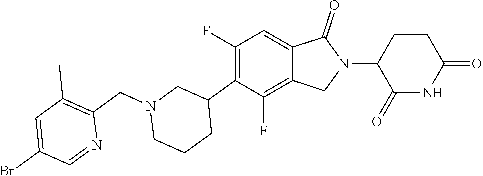 [SMILES: Cc1cc(Br)cnc1CN1CCCC(c2c(F)cc3c(c2F)CN(C2CCC(=O)NC2=O)C3=O)C1] | 3-(5-(1-((5-bromo-3- methylpyridin-2- yl)methyl)piperidin-3-yl)- 4,6-difluoro-1-oxoisoindolin- 2-yl)piperidine-2,6-dione |
| 69 | 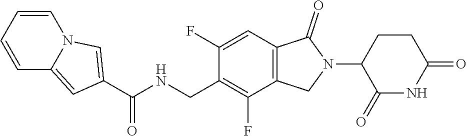 [SMILES: O=C1CCC(N2Cc3c(cc(F)c(CNC(=O)c4cc5ccccn5c4)c3F)C2=O)C(=O)N1] | N-((2-(2,6-dioxopiperidin-3- yl)-4,6-difluoro-1- oxoisoindolin-5- yl)methyl)indolizine-2- carboxamide |
| 70 | 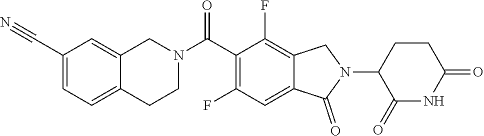 [SMILES: N#Cc1ccc2c(c1)CN(C(=O)c1c(F)cc3c(c1F)CN(C1CCC(=O)NC1=O)C3=O)CC2] | 2-(2-(2,6-dioxopiperidin-3- yl)-4,6-difluoro-1- oxoisoindoline-5-carbonyl)- 1,2,3,4- tetrahydroisoquinoline-7- carbonitrile |
| 71 | 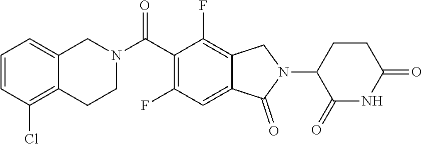 [SMILES: O=C1CCC(N2Cc3c(cc(F)c(C(=O)N4CCc5c(Cl)cccc5C4)c3F)C2=O)C(=O)N1] | 3-(5-(5-chloro-1,2,3,4- tetrahydroisoquinoline-2- carbonyl)-4,6-difluoro-1- oxoisoindolin-2- yl)piperidine-2,6-dione |
| 72 | 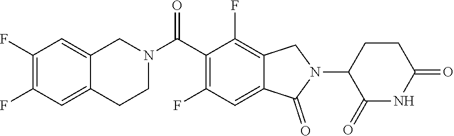 [SMILES: O=C1CCC(N2Cc3c(cc(F)c(C(=O)N4CCc5cc(F)c(F)cc5C4)c3F)C2=O)C(=O)N1] | 3-(5-(6,7-difluoro-1,2,3,4- tetrahydroisoquinoline-2- carbonyl)-4,6-difluoro-1- oxoisoindolin-2- yl)piperidine-2,6-dione |
| 73 | 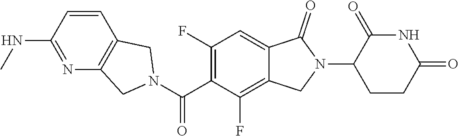 [SMILES: CNc1ccc2c(n1)CN(C(=O)c1c(F)cc3c(c1F)CN(C1CCC(=O)NC1=O)C3=O)C2] | 3-(4,6-difluoro-5-(2- (methylamino)-6,7-dihydro- 5H-pyrrolo[3,4-b]pyridine-6- carbonyl)-1-oxoisoindolin-2- yl)piperidine-2,6-dione |
| 74 | 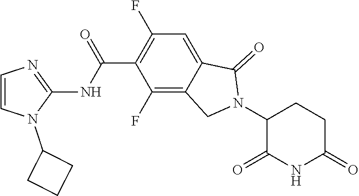 [SMILES: O=C1CCC(N2Cc3c(cc(F)c(C(=O)Nc4nccn4C4CCC4)c3F)C2=O)C(=O)N1] | N-(1-cyclobutyl-1H- imidazol-2-yl)-2-(2,6- dioxopiperidin-3-yl)-4,6- difluoro-1-oxoisoindoline-5- carboxamide |
| 75 | 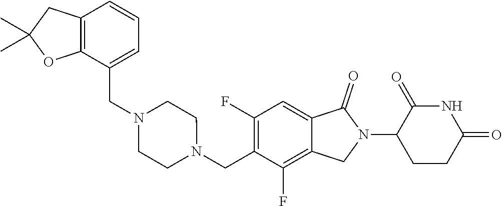 [SMILES: CC1(C)Cc2cccc(CN3CCN(Cc4c(F)cc5c(c4F)CN(C4CCC(=O)NC4=O)C5=O)CC3)c2O1] | 3-(5-((4-((2,2-dimethyl-2,3- dihydrobenzofuran-7- yl)methyl)piperazin-1- yl)methyl)-4,6-difluoro-1- oxoisoindolin-2- yl)piperidine-2,6-dione |
| 76 | 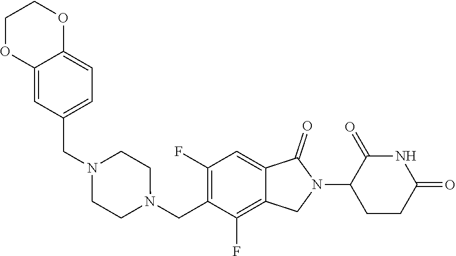 [SMILES: O=C1CCC(N2Cc3c(cc(F)c(CN4CCN(Cc5ccc6c(c5)OCCO6)CC4)c3F)C2=O)C(=O)N1] | 3-(5-((4-((2,3- dihydrobenzo[b][1,4]dioxin- 6-yl)methyl)piperazin-1- yl)methyl)-4,6-difluoro-1- oxoisoindolin-2- yl)piperidine-2,6-dione |
| 77 | 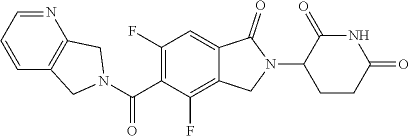 [SMILES: O=C1CCC(N2Cc3c(cc(F)c(C(=O)N4Cc5cccnc5C4)c3F)C2=O)C(=O)N1] | 3-(5-(6,7-dihydro-5H- pyrrolo[3,4-b]pyridine-6- carbonyl)-4,6-difluoro-1- oxoisoindolin-2- yl)piperidine-2,6-dione |
| 78 | 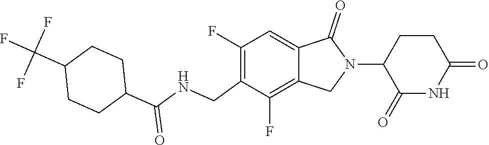 [SMILES: O=C1CCC(N2Cc3c(cc(F)c(CNC(=O)C4CCC(C(F)(F)F)CC4)c3F)C2=O)C(=O)N1] | N-((2-(2,6-dioxopiperidin-3- yl)-4,6-difluoro-1- oxoisoindolin-5-yl)methyl)- 4- (trifluoromethyl)cyclohexane- 1-carboxamide |
| 79 | 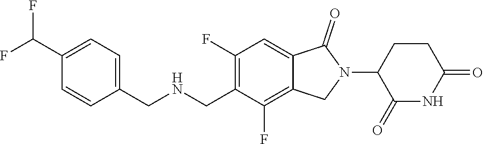 [SMILES: O=C1CCC(N2Cc3c(cc(F)c(CNCc4ccc(C(F)F)cc4)c3F)C2=O)C(=O)N1] | 3-(5-(((4- (difluoromethyl)benzyl) amino)methyl)-4,6-difluoro-1- oxoisoindolin-2- yl)piperidine-2,6-dione |
| 80 | 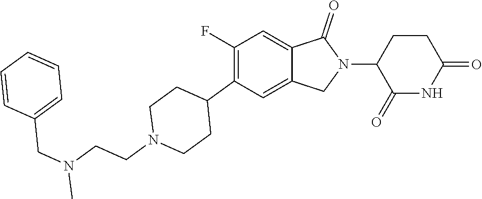 [SMILES: CN(CCN1CCC(c2cc3c(cc2F)C(=O)N(C2CCC(=O)NC2=O)C3)CC1)Cc1ccccc1] | 3-(5-(1-(2- (benzyl(methyl)amino)ethyl) piperidin-4-yl)-6-fluoro-1- oxoisoindolin-2- yl)piperidine-2,6-dione |
| 81 | 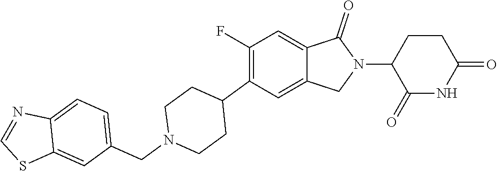 [SMILES: O=C1CCC(N2Cc3cc(C4CCN(Cc5ccc6ncsc6c5)CC4)c(F)cc3C2=O)C(=O)N1] | 3-(5-(1-(benzo[d]thiazol-6- ylmethyl)piperidin-4-yl)-6- fluoro-1-oxoisoindolin-2- yl)piperidine-2,6-dione |
| 82 | 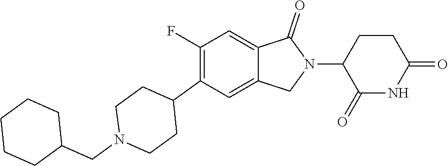 [SMILES: O=C1CCC(N2Cc3cc(C4CCN(CC5CCCCC5)CC4)c(F)cc3C2=O)C(=O)N1] | 3-(5-(1- (cyclohexylmethyl)piperidin- 4-yl)-6-fluoro-1- oxoisoindolin-2- yl)piperidine-2,6-dione |
| 83 | 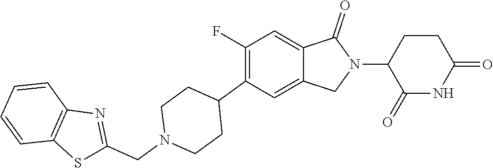 [SMILES: O=C1CCC(N2Cc3cc(C4CCN(Cc5nc6ccccc6s5)CC4)c(F)cc3C2=O)C(=O)N1] | 3-(5-(1-(benzo[d]thiazol-2- ylmethyl)piperidin-4-yl)-6- fluoro-1-oxoisoindolin-2- yl)piperidine-2,6-dione |
| 84 | 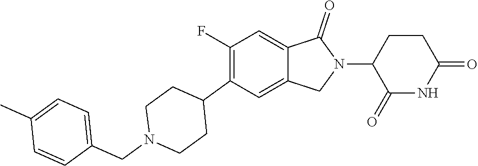 [SMILES: Cc1ccc(CN2CCC(c3cc4c(cc3F)C(=O)N(C3CCC(=O)NC3=O)C4)CC2)cc1] | 3-(6-fluoro-5-(1-(4- methylbenzyl)piperidin-4- yl)-1-oxoisoindolin-2- yl)piperidine-2,6-dione |
| 85 | 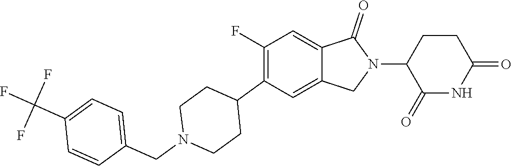 [SMILES: O=C1CCC(N2Cc3cc(C4CCN(Cc5ccc(C(F)(F)F)cc5)CC4)c(F)cc3C2=O)C(=O)N1] | 3-(6-fluoro-1-oxo-5-(1-(4- (trifluoromethyl)benzyl) piperidin-4-yl)isoindolin-2- yl)piperidine-2,6-dione |
| 86 | 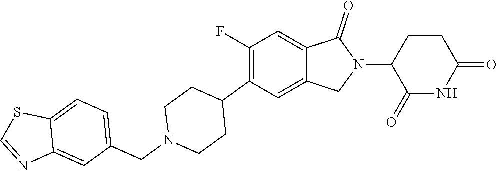 [SMILES: O=C1CCC(N2Cc3cc(C4CCN(Cc5ccc6scnc6c5)CC4)c(F)cc3C2=O)C(=O)N1] | 3-(5-(1-(benzo[d]thiazol-5- ylmethyl)piperidin-4-yl)-6- fluoro-1-oxoisoindolin-2- yl)piperidine-2,6-dione |
| 87 | 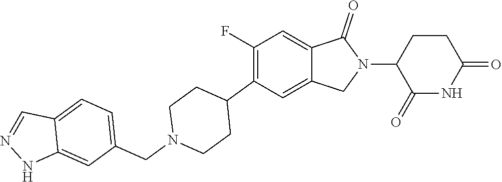 [SMILES: O=C1CCC(N2Cc3cc(C4CCN(Cc5ccc6cn[nH]c6c5)CC4)c(F)cc3C2=O)C(=O)N1] | 3-(5-(1-((1H-indazol-6- yl)methyl)piperidin-4-yl)-6- fluoro-1-oxoisoindolin-2- yl)piperidine-2,6-dione |
| 88 | 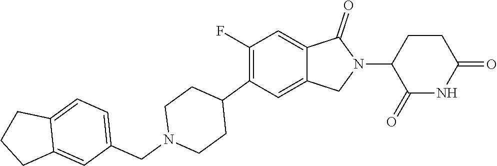 [SMILES: O=C1CCC(N2Cc3cc(C4CCN(Cc5ccc6c(c5)CCC6)CC4)c(F)cc3C2=O)C(=O)N1] | 3-(5-(1-((2,3-dihydro-1H- inden-5-yl)methyl)piperidin- 4-yl)-6-fluoro-1- oxoisoindolin-2- yl)piperidine-2,6-dione |
| 89 | 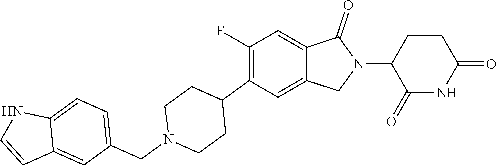 [SMILES: O=C1CCC(N2Cc3cc(C4CCN(Cc5ccc6[nH]ccc6c5)CC4)c(F)cc3C2=O)C(=O)N1] | 3-(5-(1-((1H-indol-5- yl)methyl)piperidin-4-yl)-6- fluoro-1-oxoisoindolin-2- yl)piperidine-2,6-dione |
| 90 | 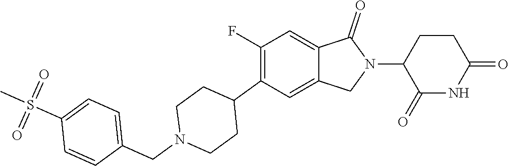 [SMILES: CS(=O)(=O)c1ccc(CN2CCC(c3cc4c(cc3F)C(=O)N(C3CCC(=O)NC3=O)C4)CC2)cc1] | 3-(6-fluoro-5-(1-(4- (methylsulfonyl)benzyl)piper idin-4-yl)-1-oxoisoindolin-2- yl)piperidine-2,6-dione |
| 91 | 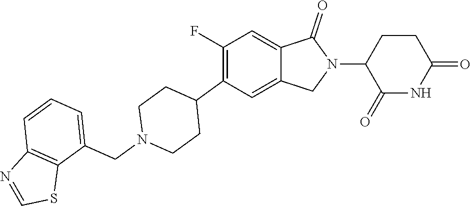 [SMILES: O=C1CCC(N2Cc3cc(C4CCN(Cc5cccc6ncsc56)CC4)c(F)cc3C2=O)C(=O)N1] | 3-(5-(1-(benzo[d]thiazol-7- ylmethyl)piperidin-4-yl)-6- fluoro-1-oxoisoindolin-2- yl)piperidine-2,6-dione |
| 92 | 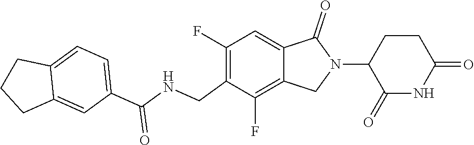 [SMILES: O=C1CCC(N2Cc3c(cc(F)c(CNC(=O)c4ccc5c(c4)CCC5)c3F)C2=O)C(=O)N1] | N-((2-(2,6-dioxopiperidin-3- yl)-4,6-difluoro-1- oxoisoindolin-5-yl)methyl)- 2,3-dihydro-1H-indene-5- carboxamide |
| 93 | 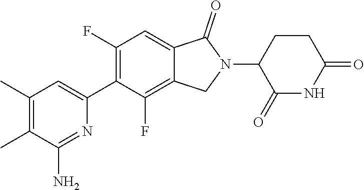 [SMILES: Cc1cc(-c2c(F)cc3c(c2F)CN(C2CCC(=O)NC2=O)C3=O)nc(N)c1C] | 3-(5-(6-amino-4,5- dimethylpyridin-2-yl)-4,6- difluoro-1-oxoisoindolin-2- yl)piperidine-2,6-dione |
| 94 | 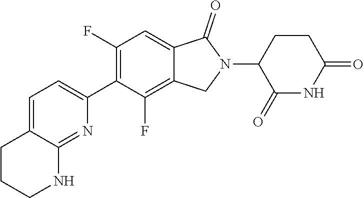 [SMILES: O=C1CCC(N2Cc3c(cc(F)c(-c4ccc5c(n4)NCCC5)c3F)C2=O)C(=O)N1] | 3-(4,6-difluoro-1-oxo-5- (5,6,7,8-tetrahydro-1,8- naphthyridin-2-yl)isoindolin- 2-yl)piperidine-2,6-dione |
| 95 | 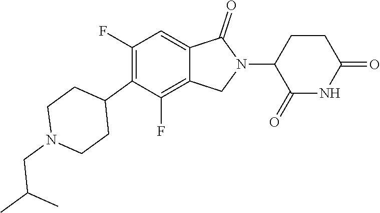 [SMILES: CC(C)CN1CCC(c2c(F)cc3c(c2F)CN(C2CCC(=O)NC2=O)C3=O)CC1] | 3-(4,6-difluoro-5-(1- isobutylpiperidin-4-yl)-1- oxoisoindolin-2- yl)piperidine-2,6-dione |
| 96 | 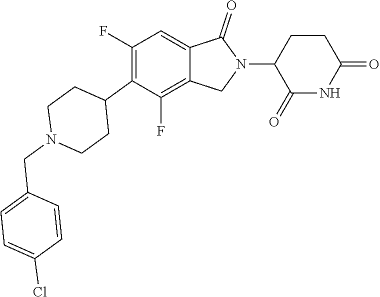 [SMILES: O=C1CCC(N2Cc3c(cc(F)c(C4CCN(Cc5ccc(Cl)cc5)CC4)c3F)C2=O)C(=O)N1] | 3-(5-(1-(4- chlorobenzyl)piperidin-4-yl)- 4,6-difluoro-1-oxoisoindolin- 2-yl)piperidine-2,6-dione |
| 97 | 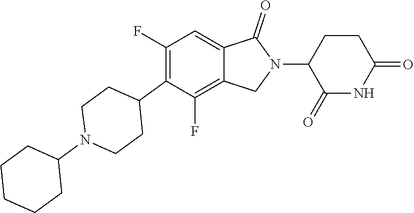 [SMILES: O=C1CCC(N2Cc3c(cc(F)c(C4CCN(C5CCCCC5)CC4)c3F)C2=O)C(=O)N1] | 3-(5-(1-cyclohexylpiperidin- 4-yl)-4,6-difluoro-1- oxoisoindolin-2- yl)piperidine-2,6-dione |
| 98 | 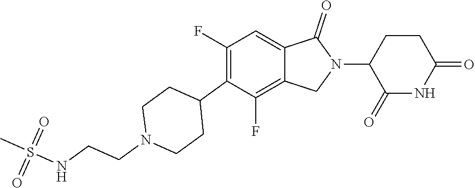 [SMILES: CS(=O)(=O)NCCN1CCC(c2c(F)cc3c(c2F)CN(C2CCC(=O)NC2=O)C3=O)CC1] | N-(2-(4-(2-(2,6- dioxopiperidin-3-yl)-4,6- difluoro-1-oxoisoindolin-5- yl)piperidin-1- yl)ethyl)methanesulfonamide |
| 99 | 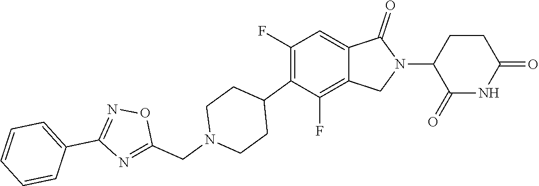 [SMILES: O=C1CCC(N2Cc3c(cc(F)c(C4CCN(Cc5nc(-c6ccccc6)no5)CC4)c3F)C2=O)C(=O)N1] | 3-(4,6-difluoro-1-oxo-5-(1- ((3-phenyl-1,2,4-oxadiazol- 5-yl)methyl)piperidin-4- yl)isoindolin-2-yl)piperidine- 2,6-dione |
| 100 | 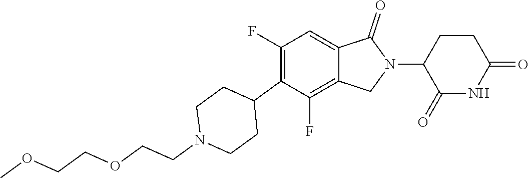 [SMILES: COCCOCCN1CCC(c2c(F)cc3c(c2F)CN(C2CCC(=O)NC2=O)C3=O)CC1] | 3-(4,6-difluoro-5-(1-(2-(2- methoxyethoxy)ethyl)piperidin- 4-yl)-1-oxoisoindolin-2- yl)piperidine-2,6-dione |
| 101 | 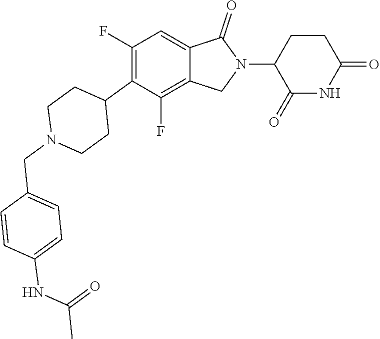 [SMILES: CC(=O)Nc1ccc(CN2CCC(c3c(F)cc4c(c3F)CN(C3CCC(=O)NC3=O)C4=O)CC2)cc1] | N-(4-((4-(2-(2,6- dioxopiperidin-3-yl)-4,6- difluoro-1-oxoisoindolin-5- yl)piperidin-1- yl)methyl)phenyl)acetamide |
| 102 | 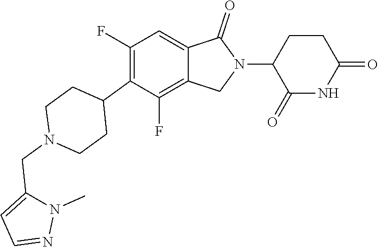 [SMILES: Cn1nccc1CN1CCC(c2c(F)cc3c(c2F)CN(C2CCC(=O)NC2=O)C3=O)CC1] | 3-(4,6-difluoro-5-(1-((1- methyl-1H-pyrazol-5- yl)methyl)piperidin-4-yl)-1- oxoisoindolin-2- yl)piperidine-2,6-dione |
| 103 | 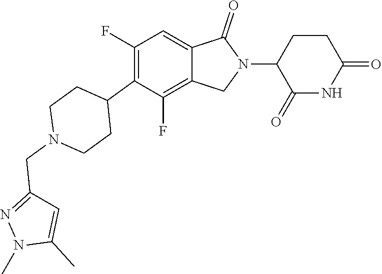 [SMILES: Cc1cc(CN2CCC(c3c(F)cc4c(c3F)CN(C3CCC(=O)NC3=O)C4=O)CC2)nn1C] | 3-(5-(1-((1,5-dimethyl-1H- pyrazol-3- yl)methyl)piperidin-4-yl)- 4,6-difluoro-1-oxoisoindolin- 2-yl)piperidine-2,6-dione |
| 104 | 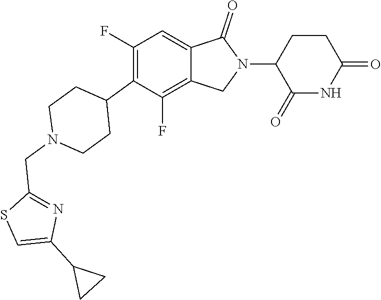 [SMILES: O=C1CCC(N2Cc3c(cc(F)c(C4CCN(Cc5nc(C6CC6)cs5)CC4)c3F)C2=O)C(=O)N1] | 3-(5-(1-((4- cyclopropylthiazol-2- yl)methyl)piperidin-4-yl)- 4,6-difluoro-1-oxoisoindolin- 2-yl)piperidine-2,6-dione |
| 105 | 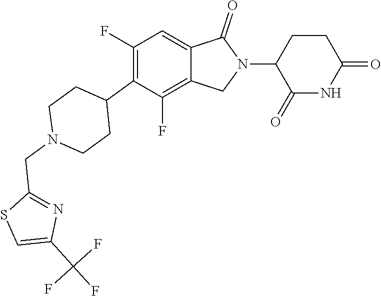 [SMILES: O=C1CCC(N2Cc3c(cc(F)c(C4CCN(Cc5nc(C(F)(F)F)cs5)CC4)c3F)C2=O)C(=O)N1] | 3-(4,6-difluoro-1-oxo-5-(1- ((4-(trifluoromethyl)thiazol- 2-yl)methyl)piperidin-4- yl)isoindolin-2-yl)piperidine- 2,6-dione |
| 106 | 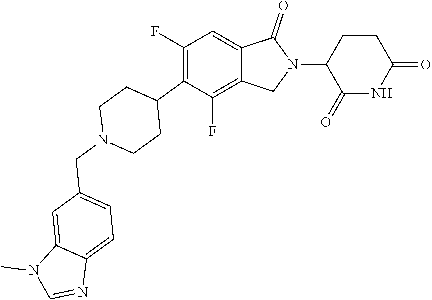 [SMILES: Cn1cnc2ccc(CN3CCC(c4c(F)cc5c(c4F)CN(C4CCC(=O)NC4=O)C5=O)CC3)cc21] | 3-(4,6-difluoro-5-(1-((1- methyl-1H- benzo[d]imidazol-6- yl)methyl)piperidin-4-yl)-1- oxoisoindolin-2- yl)piperidine-2,6-dione |
| 107 | 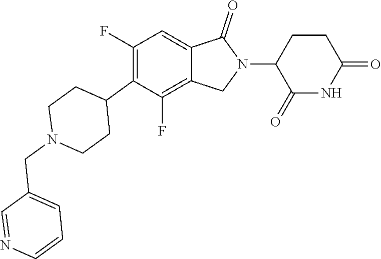 [SMILES: O=C1CCC(N2Cc3c(cc(F)c(C4CCN(Cc5cccnc5)CC4)c3F)C2=O)C(=O)N1] | 3-(4,6-difluoro-1-oxo-5-(1- (pyridin-3- ylmethyl)piperidin-4- yl)isoindolin-2-yl)piperidine- 2,6-dione |
| 108 | 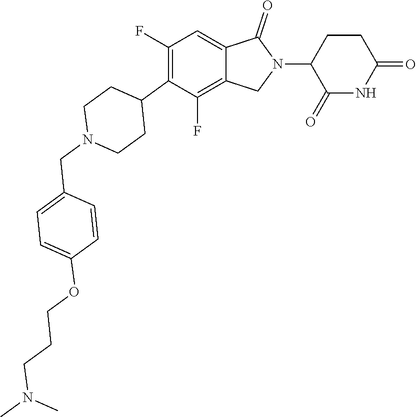 [SMILES: CN(C)CCCOc1ccc(CN2CCC(c3c(F)cc4c(c3F)CN(C3CCC(=O)NC3=O)C4=O)CC2)cc1] | 3-(5-(1-(4-(3- (dimethylamino)propoxy) benzyl)piperidin-4-yl)-4,6- difluoro-1-oxoisoindolin-2- yl)piperidine-2,6-dione |
| 109 | 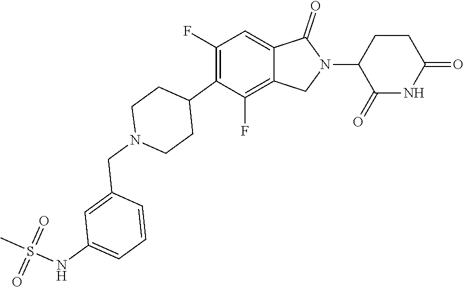 [SMILES: CS(=O)(=O)Nc1cccc(CN2CCC(c3c(F)cc4c(c3F)CN(C3CCC(=O)NC3=O)C4=O)CC2)c1] | N-(3-((4-(2-(2,6- dioxopiperidin-3-yl)-4,6- difluoro-1-oxoisoindolin-5- yl)piperidin-1- yl)methyl)phenyl) methanesulfonamide |
| 110 | 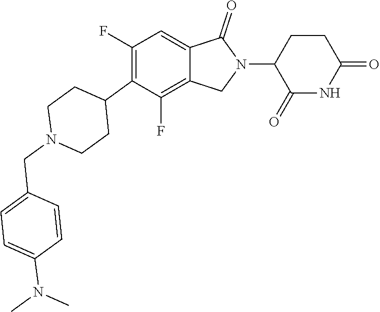 [SMILES: CN(C)c1ccc(CN2CCC(c3c(F)cc4c(c3F)CN(C3CCC(=O)NC3=O)C4=O)CC2)cc1] | 3-(5-(1-(4- (dimethylamino)benzyl) piperidin-4-yl)-4,6-difluoro-1- oxoisoindolin-2- yl)piperidine-2,6-dione |
| 111 | 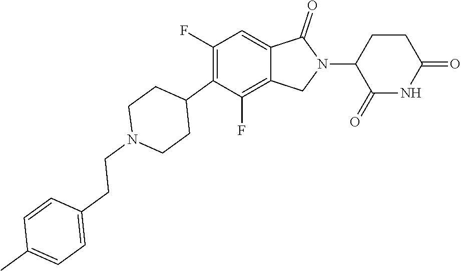 [SMILES: Cc1ccc(CCN2CCC(c3c(F)cc4c(c3F)CN(C3CCC(=O)NC3=O)C4=O)CC2)cc1] | 3-(4,6-difluoro-5-(1-(4- methylphenethyl)piperidin- 4-yl)-1-oxoisoindolin-2- yl)piperidine-2,6-dione |
| 112 | 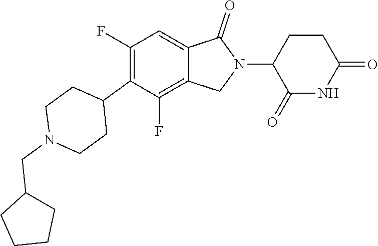 [SMILES: O=C1CCC(N2Cc3c(cc(F)c(C4CCN(CC5CCCC5)CC4)c3F)C2=O)C(=O)N1] | 3-(5-(1- (cyclopentylmethyl)piperidin- 4-yl)-4,6-difluoro-1- oxoisoindolin-2- yl)piperidine-2,6-dione |
| 113 | 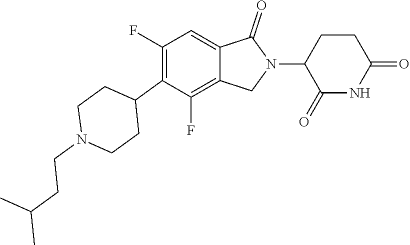 [SMILES: CC(C)CCN1CCC(c2c(F)cc3c(c2F)CN(C2CCC(=O)NC2=O)C3=O)CC1] | 3-(4,6-difluoro-5-(1- isopentylpiperidin-4-yl)-1- oxoisoindolin-2- yl)piperidine-2,6-dione |
| 114 | 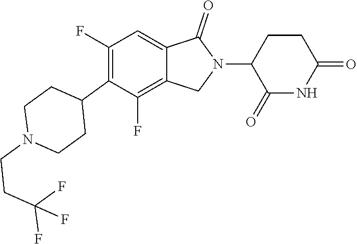 [SMILES: O=C1CCC(N2Cc3c(cc(F)c(C4CCN(CCC(F)(F)F)CC4)c3F)C2=O)C(=O)N1] | 3-(4,6-difluoro-1-oxo-5-(1- (3,3,3- trifluoropropyl)piperidin-4- yl)isoindolin-2-yl)piperidine- 2,6-dione |
| 115 | 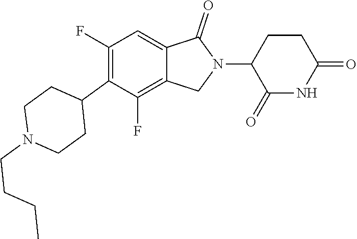 [SMILES: CCCCN1CCC(c2c(F)cc3c(c2F)CN(C2CCC(=O)NC2=O)C3=O)CC1] | 3-(5-(1-butylpiperidin-4-yl)- 4,6-difluoro-1-oxoisoindolin- 2-yl)piperidine-2,6-dione |
| 116 | 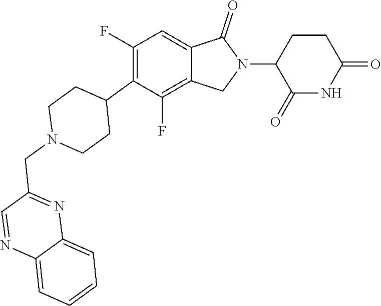 [SMILES: O=C1CCC(N2Cc3c(cc(F)c(C4CCN(Cc5cnc6ccccc6n5)CC4)c3F)C2=O)C(=O)N1] | 3-(4,6-difluoro-1-oxo-5-(1- (quinoxalin-2- ylmethyl)piperidin-4- yl)isoindolin-2-yl)piperidine- 2,6-dione |
| 117 | 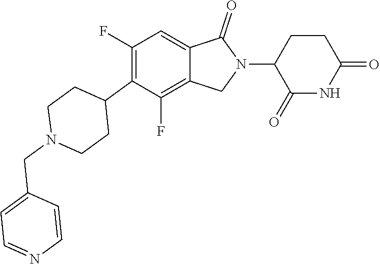 [SMILES: O=C1CCC(N2Cc3c(cc(F)c(C4CCN(Cc5ccncc5)CC4)c3F)C2=O)C(=O)N1] | 3-(4,6-difluoro-1-oxo-5-(1- (pyridin-4- ylmethyl)piperidin-4- yl)isoindolin-2-yl)piperidine- 2,6-dione |
| 118 | 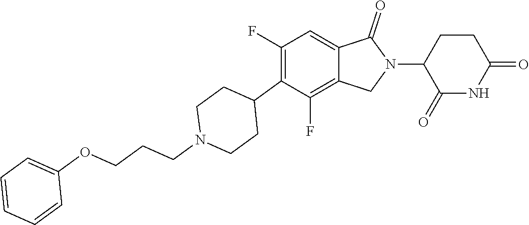 [SMILES: O=C1CCC(N2Cc3c(cc(F)c(C4CCN(CCCOc5ccccc5)CC4)c3F)C2=O)C(=O)N1] | 3-(4,6-difluoro-1-oxo-5-(1- (3-phenoxypropyl)piperidin- 4-yl)isoindolin-2- yl)piperidine-2,6-dione |
| 119 | 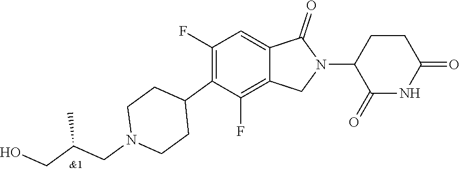 [SMILES: C[C@@H](CO)CN1CCC(c2c(F)cc3c(c2F)CN(C2CCC(=O)NC2=O)C3=O)CC1] | rac-3-(4,6-difluoro-5-(1- ((R)-3-hydroxy-2- methylpropyl)piperidin-4- yl)-1-oxoisoindolin-2- yl)piperidine-2,6-dione |
| 120 | 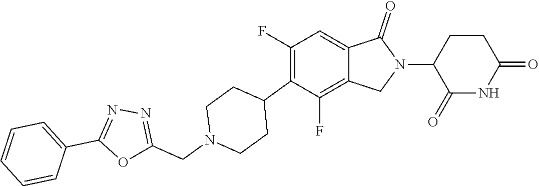 [SMILES: O=C1CCC(N2Cc3c(cc(F)c(C4CCN(Cc5nnc(-c6ccccc6)o5)CC4)c3F)C2=O)C(=O)N1] | 3-(4,6-difluoro-1-oxo-5-(1- ((5-phenyl-1,3,4-oxadiazol- 2-yl)methyl)piperidin-4- yl)isoindolin-2-yl)piperidine- 2,6-dione |
| 121 | 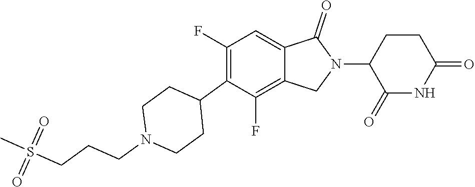 [SMILES: CS(=O)(=O)CCCN1CCC(c2c(F)cc3c(c2F)CN(C2CCC(=O)NC2=O)C3=O)CC1] | 3-(4,6-difluoro-5-(1-(3- (methylsulfonyl)propyl)piper idin-4-yl)-1-oxoisoindolin-2- yl)piperidine-2,6-dione |
| 122 | 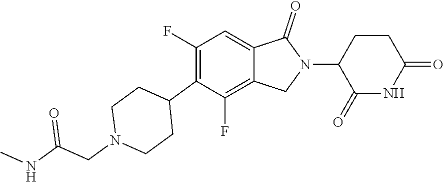 [SMILES: CNC(=O)CN1CCC(c2c(F)cc3c(c2F)CN(C2CCC(=O)NC2=O)C3=O)CC1] | 2-(4-(2-(2,6-dioxopiperidin- 3-yl)-4,6-difluoro-1- oxoisoindolin-5-yl)piperidin- 1-yl)-N-methylacetamide |
| 123 | 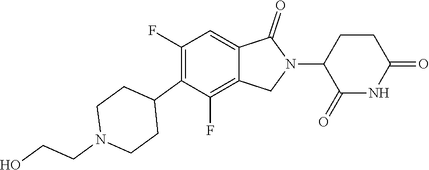 [SMILES: O=C1CCC(N2Cc3c(cc(F)c(C4CCN(CCO)CC4)c3F)C2=O)C(=O)N1] | 3-(4,6-difluoro-5-(1-(2- hydroxyethyl)piperidin-4- yl)-1-oxoisoindolin-2- yl)piperidine-2,6-dione |
| 124 | 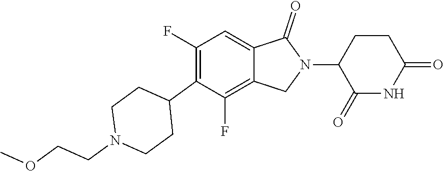 [SMILES: COCCN1CCC(c2c(F)cc3c(c2F)CN(C2CCC(=O)NC2=O)C3=O)CC1] | 3-(4,6-difluoro-5-(1-(2- methoxyethyl)piperidin-4- yl)-1-oxoisoindolin-2- yl)piperidine-2,6-dione |
| 125 | 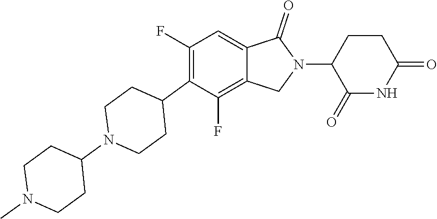 [SMILES: CN1CCC(N2CCC(c3c(F)cc4c(c3F)CN(C3CCC(=O)NC3=O)C4=O)CC2)CC1] | 3-(4,6-difluoro-5-(1′-methyl- [1,4′-bipiperidin]-4-yl)-1- oxoisoindolin-2- yl)piperidine-2,6-dione |
| 126 | 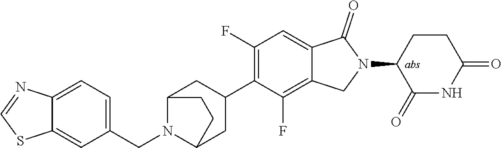 [SMILES: O=C1CC[C@H](N2Cc3c(cc(F)c(C4CC5CCC(C4)N5Cc4ccc5ncsc5c4)c3F)C2=O)C(=O)N1] | (3S)-3-(5-(8- (benzo[d]thiazol-6- ylmethyl)-8- azabicyclo[3.2.1]octan-3-yl)- 4,6-difluoro-1-oxoisoindolin- 2-yl)piperidine-2,6-dione |
| 127 | 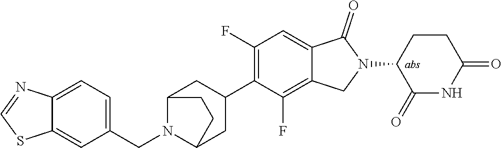 [SMILES: O=C1CC[C@@H](N2Cc3c(cc(F)c(C4CC5CCC(C4)N5Cc4ccc5ncsc5c4)c3F)C2=O)C(=O)N1] | (3R)-3-(5-(8- (benzo[d]thiazol-6- ylmethyl)-8- azabicyclo[3.2.1]octan-3-yl)- 4,6-difluoro-1-oxoisoindolin- 2-yl)piperidine-2,6-dione |
| 128 | 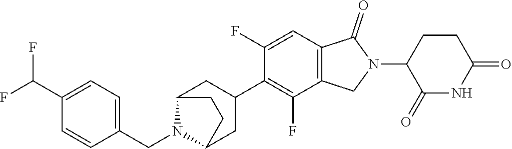 [SMILES: O=C1CCC(N2Cc3c(cc(F)c(C4C[C@H]5CC[C@@H](C4)N5Cc4ccc(C(F)F)cc4)c3F)C2=O)C(=O)N1] | 3-(5-((1R,5S)-8-(4- (difluoromethyl)benzyl)-8- azabicyclo[3.2.1]octan-3-yl)- 4,6-difluoro-1-oxoisoindolin- 2-yl)piperidine-2,6-dione |

&1 represents a stereocenter.
abs represents a chiral center.

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in General Schemes 1-4, as well as in the examples set forth herein. It should be noted that one skilled in the art would know how to modify the procedures set forth in the illustrative schemes and examples to arrive at the desired products.

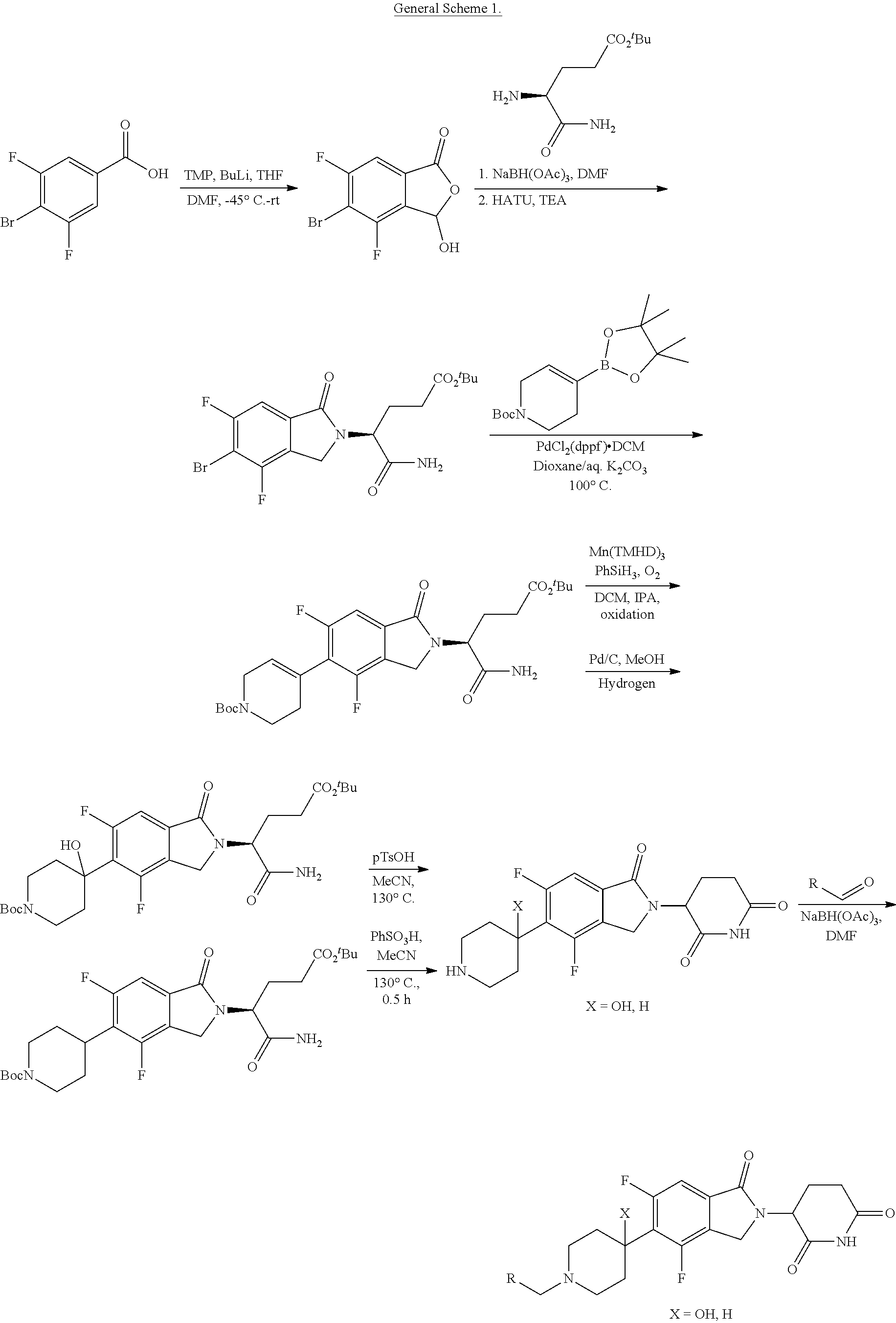

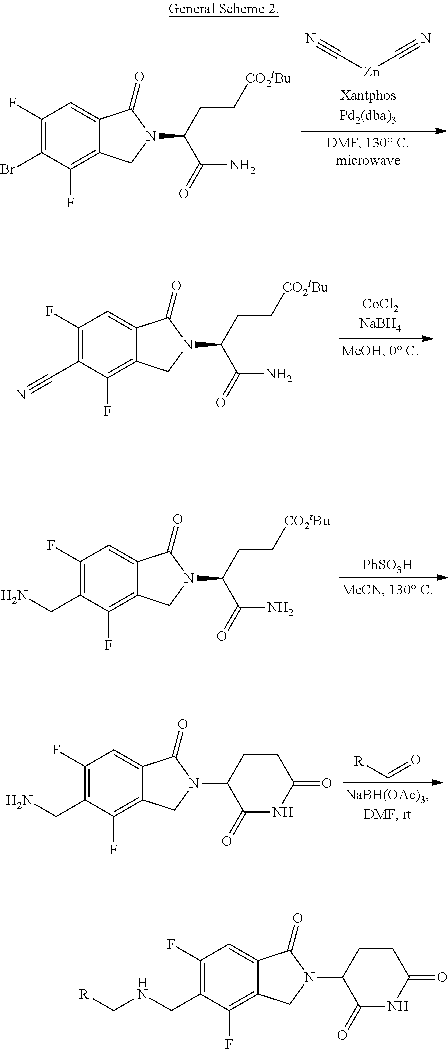

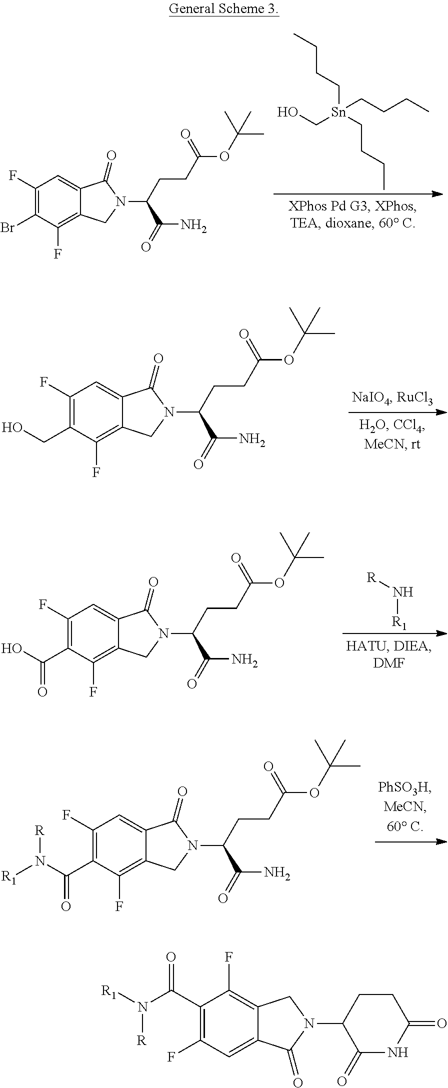

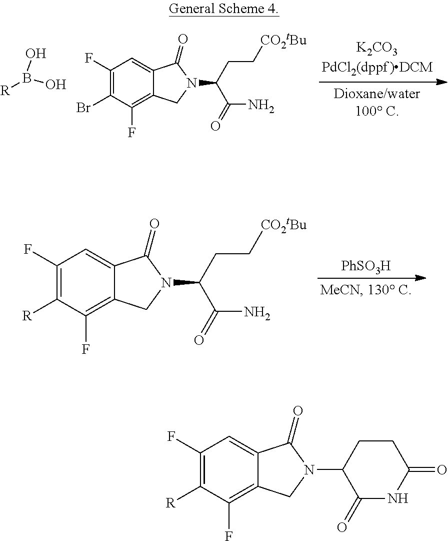

### Methods of Use

Embodiments of the present disclosure provide a method for degrading an engineered polypeptide in a cell, a method for reducing engineered polypeptide levels in a cell, and a method of treating diseases such as cancer in a subject in need thereof.

In some embodiments, a method of reducing the level of an engineered polypeptide comprising a degradation domain is provided, comprising contacting the engineered polypeptide with a compound of Formula (I). In some embodiments, the contacting occurs in a cell, and the a compound of Formula (I) binds to the degradation domain and a ubiquitin ligase, resulting in ubiquitination and degradation of the engineered polypeptide. In some embodiments, degradation of the engineered polypeptide results in a decrease of at least one activity of the cell and/or an increase of at least one activity of the cell and/or death of the cell. Nonlimiting exemplary effects include lowing the threshold for cell (such as T cell) activation, increasing functional persistence of the cell (such as a T cell), promoting survival of the cell, and increased proliferation of the cell. In some embodiments, the method comprises administering ta compound of Formula (I) to a subject, wherein the subject comprises cells that comprise the engineered polypeptide.

In some embodiments, the engineered polypeptide is degraded in the presence of a compound of Formula (I). In some embodiments, a compound of Formula (I) interacts with the degradation domain and with a ubiquitin ligase, such as cereblon. In some embodiments, a compound of Formula (I) mediates a complex comprising the degradation domain, a compound of Formula (I), and the ubiquitin ligase, resulting in ubiquitination of the engineered polypeptide.

The modified cells provided herein, such as T lymphocytes (i.e., T cells) modified to comprise/express an engineered polypeptide (e.g., CAR cells), can be used to treat an individual who would benefit from the modified cells, for example, because the individual has a cancer that expresses a target of a CAR. In some embodiments, the cell is a T effector cell. In some embodiments, the cell is a CD4+ T cell or a CD8+ T cell. In some embodiments, either the T cell, T effector cell, CD4+ T cell or a CD8+ T cell comprises the engineered polypeptide.

In one aspect, provided herein is a method for degrading an engineered polypeptide comprising a degradation domain in a cell in a subject in need thereof, the method comprising contacting a cell with an effective amount of a compound of Formula (I). Degradation of the engineered polypeptide in a cell can be assessed and demonstrated by a wide variety of methods known in the art. Kits and commercially available assays, including cell-based assays, can be utilized for determining whether and to what degree the engineered polypeptide in a cell has been degraded. In some embodiments, the compound of Formula (I) partially degrades an engineered polypeptide in a cell. In some embodiments, the compound of Formula (I) fully degrades an engineered polypeptide in a cell.

In some embodiments, a compound of Formula (I) degrades an engineered polypeptide in a cell by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades an engineered polypeptide in a cell by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, provided herein is a method for reducing engineered polypeptide levels in a cell, the method comprising contacting a cell with an effective amount of a compound of Formula (I). Reduction of engineered polypeptide levels in a cell can be assessed and demonstrated by a wide variety of methods known in the art. Kits and commercially available assays, including cell-based assays, can be utilized for determining whether and to what degree kinase protein levels have been reduced.

In some embodiments, a compound of Formula (I) reduces engineered polypeptide levels in a cell by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) reduces engineered polypeptide levels in a cell by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, a compound of Formula (I) has an EC₅₀ value as measured in a engineered polypeptide degradation assay of from about 0.0003 μM to about 1 μM or from about 0.0003 μM to about 0.2 μM or from about 0.0003 μM to about 0.05 μM. In some embodiments, a compound of Formula (I) has an EC₅₀ of from about 0.05 μM to about 0.2 μM. In some embodiments, a compound of Formula (I) has an EC₅₀ of from about 0.2 μM to about 1 μM. In some embodiments, a compound of Formula (I) has an EC50 of less than about 1 μM. In some embodiments, a compound of Formula (I) has an EC50 value of less than 0.2 μM, less than 0.05 μM, less than 0.001 μM, or less than about 0.0003 μM.

In some embodiments, following administration of a CAR cell comprising an engineered polypeptide comprising a degradation domain, it may be desirable to reduce or eliminate expression of the CAR and thus reduce or eliminate targeted cell killing. In some such embodiments, the method may further comprise administering a compound of Formula (I) to the subject. Administration of a compound of Formula (I) results in degradation of the engineered polypeptide (e.g., the CAR), and reduces or eliminates targeting of the modified cells to cells expressing the antigen bound by the antigen-binding domain of the CAR. In this way, the activity of treatments with CAR cells may be modulated, and safety may be improved.

In some embodiments, said population of modified cells is administered first to the subject, followed by administration of a compound of Formula (I) at a specified period of time after administration of the modified cell population, e.g., 30 minutes, 1 hour, 6 hours, 12 hours, 1 day, 2 days, 3 days, 4 days, 5 days, 6 days, or 1 week after administration of the cell population.

A non-limiting list of cancers that can be treated in accordance with the methods of treatment described herein includes lymphoma, leukemia, lung cancer, breast cancer, prostate cancer, adrenocortical carcinoma, thyroid carcinoma, nasopharyngeal carcinoma, melanoma, skin carcinoma, colorectal carcinoma, desmoid tumor, aesmoplastic small round cell tumor, endocrine tumor, Ewing sarcoma, peripheral primitive neuroectodermal tumor, solid germ cell tumor, hepatoblastoma, neuroblastoma, non-rhabdomyosarcoma soft tissue sarcoma, osteosarcoma, retinoblastoma, rhabdomyosarcoma, Wilms tumor, glioma, glioblastoma, myxoma, fibroma, and lipoma. Exemplary lymphomas and leukemias include, without limitation, chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma.

Efficacy of the modified cells described herein, such as CAR cells, in treatment of a disease or disorder, e.g., in treatment of an individual having cancer, can be assessed by one or more criteria specific to the particular disease or disorder, known to those of ordinary skill in the art, to be indicative of progress of the disease or disorder. Generally, administration of CAR cells (e.g., CAR T lymphocytes) to an individual having a disease/disorder (e.g., cancer) is effective when one or more of said criteria detectably, e.g., significantly, moves from a disease state value or range to, or towards, a normal value or range.

In some embodiments, compounds of Formula (I) are useful in the manufacture of a medicament for reducing engineered polypeptide levels in a cell.

The methods and uses of the present disclosure may include a compound of Formula (I) used alone or in combination with one or more additional therapies (e.g., non-drug treatments or therapeutic agents).

A compound of Formula (I) may be administered before, after, or concurrently with one or more of such additional therapies. When combined, dosages of the compound of Formula (I) and dosages of the one or more additional therapies (e.g., non-drug treatment or therapeutic agent) may provide a therapeutic effect (e.g., synergistic or additive therapeutic effect). A compound of Formula (I) and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment). For example, in some embodiments, the compounds of Formula (I) can be used in combination with a therapeutic agent that treats nausea. Examples of agents that can be used to treat nausea include, but are not limited to, dronabinol, granisetron, metoclopramide, ondansetron, prochlorperazine, and pharmaceutically acceptable salts thereof.

In some embodiments, one or more additional therapies includes a non-drug treatment (e.g., surgery or radiation therapy). In some embodiments, one or more additional therapies includes a therapeutic agent (e.g., a compound or biologic that is an antiproliferative agent). In some embodiments, one or more additional therapies includes a non-drug treatment and a therapeutic agent. In other embodiments, one or more additional therapies includes two therapeutic agents. In still other embodiments, one or more additional therapies includes three therapeutic agents. In some embodiments, one or more additional therapies includes four or more therapeutic agents.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In any given case, the amount of the compound of Formula (I) administered will depend on such factors as the solubility of the active component, the formulation used and the route of administration.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

EtOAc: Ethyl acetate
DCM: Dichloromethane
DMF: Dimethylformamide
DMSO: Dimethyl sulfoxide
MeCN: Acetonitrile
THF: Tetrahydrofuran
MeOH: Methanol
EtOH: Ethanol
AcOH: Acetic acid
TFA: Trifluoroacetic acid
NaBH₄: Sodium borohydride
NaBH(OAc)₃: Sodium triacetoxyborohydride
DIPEA: N,N-Diisopropylethylamine
TEA: Triethylamine
TMP: 2,2,6,6-Tetramethylpiperidine
n-BuLi: n-Butyllithium
N₂: Nitrogen
Na₂SO₄: Sodium sulfate
MgSO₄: Magnesium sulfate
PdCl₂(dtbpf): [1,1′-Bis(di-tert-butylphosphino)ferrocene]dichloropalladium (II)
Pd(dppf)Cl₂: [1,1′-Bis(diphenylphosphino)ferrocene]dichloropalladium (II)
Pd(dppf)Cl₂·DCM: [1,1′-Bis(diphenylphosphino)ferrocene]dichloropalladium (II) complex with dichloromethane
Pd₂(dba)₃: Tris(dibenzylideneacetone) dipalladium
Pd/C: Palladium on carbon
HATU: (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium)

### SYNTHETIC EXAMPLES

### Analytical Methods

Preparative HPLC Method 1: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 15% B, 15-50% B over 25 min, then a 6-min hold at 100% B; Flow Rate: 20 mL/min; Column Temperature: 25° C. Fraction collection was triggered by MS signals.

Preparative HPLC Method 2:1-Phen Luna Axia C18 5u 30×100 mm; Mobile Phase A: 95% H₂O/5% ACN/0.05% TFA; Mobile Phase B: 5% H₂O/95% ACN/0.05% TFA; Gradient: a 0-min hold at 2% B, 2-100% B over 12 min, then a 5-min hold at 100% B; Flow Rate: 25 mL/min; Column Temperature: 25° C. Fraction collection was triggered by UV (220) nm.

Analytical HPLC Method 1: Waters XBridge C18, 2.1 mm×50 mm, 1.7 μm particles; Mobile Phase A: 5:95 acetonitrile: water with 0.1% trifluoroacetic acid; Mobile Phase B: 95:5 acetonitrile: water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm).

### Example S1. 3-(4,6-difluoro-5-(4-hydroxy-1-(4-(trifluoromethyl)benzyl) piperidin-4-yl)-1-oxoisoindolin-2-yl) piperidine-2,6-dione (Compound No 11)

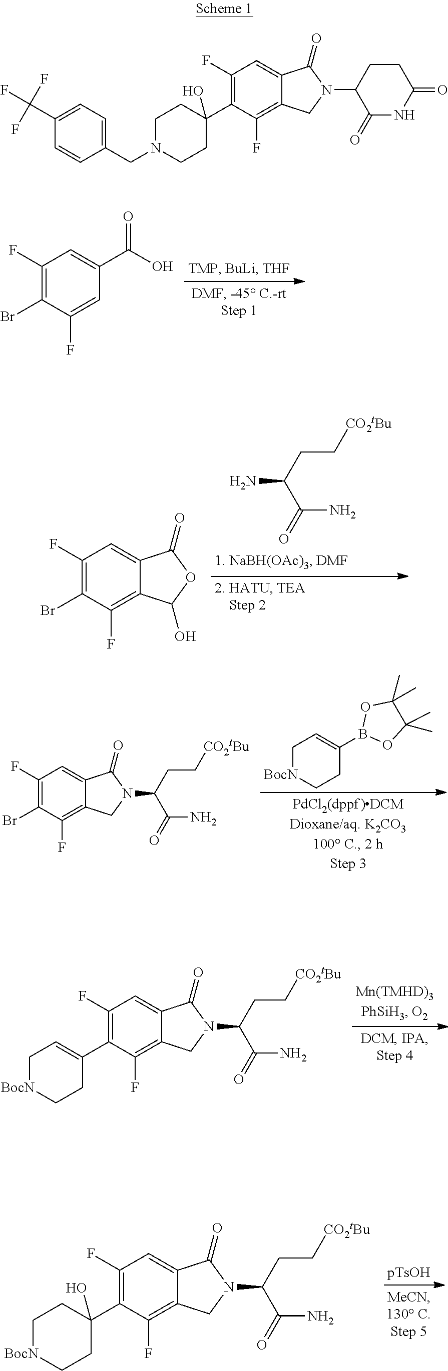

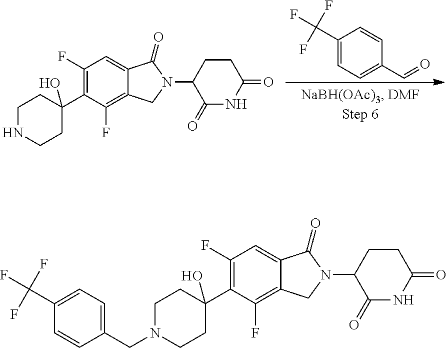 [SMILES: O=C1CCC(N2Cc3c(cc(F)c(C4(O)CCNCC4)c3F)C2=O)C(=O)N1 ; O=Cc1ccc(C(F)(F)F)cc1 ; O=C1CCC(N2Cc3c(cc(F)c(C4(O)CCN(Cc5ccc(C(F)(F)F)cc5)CC4)c3F)C2=O)C(=O)N1]

Step 1: To a stirred solution of 2,2,6,6-tetramethylpiperidine (17.95 mL, 105 mmol) in THF (150 mL) at 0° C. under an atmosphere of nitrogen was added n-BuLi (63.3 mL, 101 mmol) dropwise, and the resulting mixture was stirred for 30 min at 0° C. The reaction mixture was then cooled to about −45° C. (using dry ice/MeCN bath) and 4-bromo-3,5-difluorobenzoic acid (10.0 g, 42.2 mmol), dissolved in THF (25 mL), was added dropwise and stirring was continued at −45° C. After 3 h, DMF (4.88 mL, 63.3 mmol) was added dropwise and the reaction mixture was allowed to warm to room temperature and stirred overnight. The reaction mixture was quenched with aq. 3M HCl (40 mL) at 0° C. and extracted with DCM (×3). The combined organic phases were dried over Na₂SO₄, filtered, and concentrated to dryness. The crude product was purified via silica gel chromatography eluting with 0-80% EtOAc/hexanes to afford 5-bromo-4,6-difluoro-3-hydroxyisobenzofuran-1 (3H)-one (4.612 g, 33.0% yield).

¹H NMR (400 MHZ, CHLOROFORM-d) δ 10.27 (s, 1H), 7.53 (dd, J=8.2, 1.7 Hz, 1H).

Step 2: To a solution 5-bromo-4,6-difluoro-3-hydroxyisobenzofuran-1 (3H)-one (4.0 g, 15.09 mmol) in DMF (100 mL) was added tert-butyl(S)-4,5-diamino-5-oxopentanoate, HCl (3.60 g, 15.09 mmol) followed by NaBH(OAc)₃ (4.80 g, 22.64 mmol). It was stirred at room temperature for 16 h. To this mixture was added HATU (7.17 g, 18.87 mmol) and triethylamine (8.42 mL, 60.4 mmol) stirred at room temperature for 2 h. The reaction was quenched by adding 10% aq. LiCl, and then the product was extracted with EtOAc. The organic layer was washed with brine and dried over MgSO₄ and then concentrated. The crude was purified using a 120 g silica gel column by ISCO, eluting with 0-80% EtOAc/hexanes to obtain(S)-5-amino-4-(5-bromo-4,6-difluoro-1-oxoisoindolin-2-yl)-5-oxopentanoate (2.01 g, 56.1% yield).

¹H NMR (400 MHZ, CHLOROFORM-d) δ ppm 7.40-7.51 (m, 1H) 6.11-6.26 (m, 1H) 5.39 (br s, 1H) 4.91 (dd, J=8.68, 6.15 Hz, 1H) 4.67-4.76 (m, 1H) 4.49-4.58 (m, 1H) 2.26-2.43 (m, 3H) 2.11-2.21 (m, 1H) 1.45 (s, 9H)

Step 3: To a solution of tert-butyl 4-(4,4,5,5-tetramethyl-1,3,2-dioxaborolan-2-yl)-3,6-dihydropyridine-1 (2H)-carboxylate (1.383 g, 4.47 mmol) and tert-butyl(S)-5-amino-4-(5-bromo-4,6-difluoro-1-oxoisoindolin-2-yl)-5-oxopentanoate (1.55 g, 3.58 mmol) in dioxane (30 mL) was added K₂CO₃ (1.236 g, 8.94 mmol) dissolved in water (15 mL). To this was added PdCl₂(dppf)·DCM (0.146 g, 0.179 mmol) and the air was replaced with nitrogen. It was heated to 100° C. for 1 h. It was cooled to room temperature, diluted with EtOAc and quenched with brine and the organic layer separated. The organic layer was washed with brine, dried over Na₂SO₄, filtered and concentrated. The crude product was purified by flash column chromatography eluting with 0-15% B/DCM [where B=15% ethanol in EtOAc+0.1% TEA].

LC/MS (ESI) m/z 536.5 [(M+H), calcd for C₂₇H₃₅F₂N₃O₆ 535.2].

Step 4: Oxygen was bubble through a solution of tert-butyl(S)-4-(2-(1-amino-5-(tert-butoxy)-1,5-dioxopentan-2-yl)-4,6-difluoro-1-oxoisoindolin-5-yl)-3,6-dihydropyridine-1 (2H)-carboxylate (1.00 g, 1.867 mmol) in DCM (10 mL) and 2-Propanol (40 mL) for 5 min. Then, tris(2,2,6,6-tetramethyl-3,5-heptanedionato) manganese (III) (0.113 g, 0.187 mmol) and phenylsilane (0.404 g, 3.73 mmol) were added. The reaction mixture was stirred under oxygen balloon at room temperature for 2 days. The reaction mixture was diluted with EtOAc and washed with sodium thiosulfate solution. The organic layer was dried and concentrated and purified by ISCO, using a 40 g silica gel column and eluting with 0-5% MeOH/DCM to give 417 mg of the desired product as a white solid.

LC/MS (ESI) m/z 498.4 [(M-55), calcd for C₂₇H₃₇F₂N₃O₇ 553.3].

Step 5: A 20 mL microwave vial was charged with (tert-butyl(S)-4-(2-(1-amino-5-(tert-butoxy)-1,5-dioxopentan-2-yl)-4,6-difluoro-1-oxoisoindolin-5-yl)-4-hydroxypiperidine-1-carboxylate (410 mg, 0.741 mmol), 4-methylbenzenesulfonic acid (255 mg, 1.481 mmol) and acetonitrile (10 mL). It was heated to 120° C. for 1 hour in the microwave. It was concentrated to dryness, and the residues rinsed with ether to remove the excess pTsOH. The precipitate was air-dried to obtain 391 mg of product as a mono-pTsOH salt.

LC/MS (ESI) m/z 380.3 [(M+H), calcd for C₁₈H₁₉F₂N₃O₄ 379.1]. The enantiomeric excess of this material and subsequent compounds were not determined.

Step 6: To a solution of 4-(trifluoromethyl)benzaldehyde (12.85 mg, 0.074 mmol) and(S)-3-(4,6-difluoro-5-(4-hydroxypiperidin-4-yl)-1-oxoisoindolin-2-yl) piperidine-2,6-dione (14 mg, 0.037 mmol) in DMF (1 mL) was added 2 drops of AcOH, and then stirred at room temperature. After 0.5 h, NaBH(OAc)₃ (23.46 mg, 0.111 mmol) was added and the resulting solution stirred at room temperature for 16 h. The reaction mixture was purified by Preparative HPLC Method 1 to obtain 7.7 mg of product.

LC/MS (ESI) m/z 538.1 [(M+H)⁺, calcd for C₂₆H₂₄F₅N₃O₄ 537.2]; HPLC^{a} T_{Ret}=1.25 min; ¹H NMR (500 MHZ, DMSO-d₆) δ 11.01 (s, 1H), 7.87-7.82 (m, 2H), 7.76 (br d, J=7.9 Hz, 2H), 7.44 (br d, J=10.4 Hz, 1H), 5.11-5.05 (m, 1H), 4.53 (br d, J=17.1 Hz, 1H), 4.41-4.35 (m, 1H), 2.95-2.85 (m, 1H), 2.62 (br d, J=17.1 Hz, 1H), 2.51 (br s, 4H), 2.47-2.33 (m, 2H), 2.25 (br d, J=13.7 Hz, 2H), 2.06-1.99 (m, 1H).

### Example S2. 3-(4,6-difluoro-1-oxo-5-(1-(4-(trifluoromethyl)benzyl) piperidin-4-yl) isoindolin-2-yl) piperidine-2,6-dione (Compound No 52)

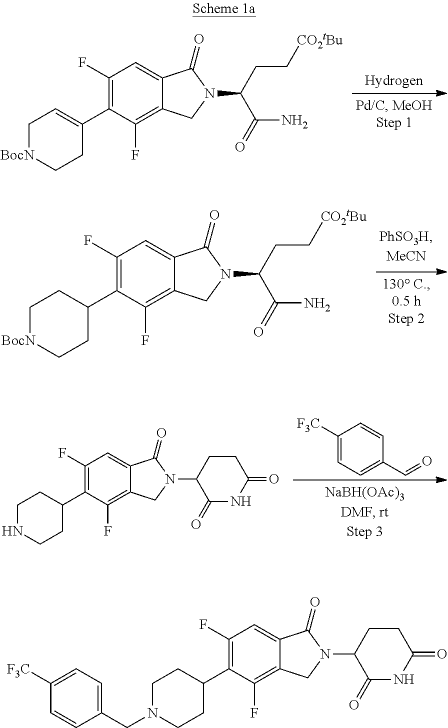 [SMILES: CC(C)(C)OC(=O)CC[C@@H](C(N)=O)N1Cc2c(cc(F)c(C3=CCN(C(=O)OC(C)(C)C)CC3)c2F)C1=O ; CC(C)(C)OC(=O)CC[C@@H](C(N)=O)N1Cc2c(cc(F)c(C3CCN(C(=O)OC(C)(C)C)CC3)c2F)C1=O ; O=C1CCC(N2Cc3c(cc(F)c(C4CCNCC4)c3F)C2=O)C(=O)N1 ; O=Cc1ccc(C(F)(F)F)cc1 ; O=C1CCC(N2Cc3c(cc(F)c(C4CCN(Cc5ccc(C(F)(F)F)cc5)CC4)c3F)C2=O)C(=O)N1]

Step 1: A 200 mL round bottom flask was charged with tert-butyl(S)-4-(2-(1-amino-5-(tert-butoxy)-1,5-dioxopentan-2-yl)-4,6-difluoro-1-oxoisoindolin-5-yl)-3,6-dihydropyridine-1 (2H)-carboxylate (1.20 g, 2.241 mmol), MeOH (50 mL) and Pd/C (0.119 g, 0.112 mmol). The air was replaced with hydrogen and stirred vigorously at room temperature for 16 h over an atmosphere of hydrogen. It was filtered through a pad of celite and concentrated to obtain the desired product.

LC/MS (ESI) m/z 538.5 [(M+H)⁺, calcd for C₂₇H₃₇F₂N₃O₆ 537.3]

Step 2: A 20 mL microwave vial was charged with tert-butyl(S)-4-(2-(1-amino-5-(tert-butoxy)-1,5-dioxopentan-2-yl)-4,6-difluoro-1-oxoisoindolin-5-yl) piperidine-1-carboxylate (770 mg, 1.432 mmol), benzenesulfonic acid (453 mg, 2.86 mmol) and MeCN (15 mL). It was heated to 130° C. for 0.5 h in the microwave. It was diluted with ether, and the precipitates were collected by filtration and then air dried to obtain a mono-PhSO₃H salt of the desired product. The TFA salt form of this intermediate was obtained after subjecting it to preparative HPLC Method 2.

MS: C₁₈H₁₉F₂N₃O₃ [M+H]⁺ 364, found [M+H]⁺ 364. ¹H NMR (300 MHz, DMSO-d₆) δ 11.02 (s, 1H), 8.65 (d, J=11.3 Hz, 1H), 8.38 (d, J=10.8 Hz, 1H), 7.74-7.55 (m, 3H), 7.50 (d, J=8.8 Hz, 1H), 7.40-7.23 (m, 5H), 5.12 (dd, J=13.3, 5.1 Hz, 1H), 4.55 (d, J=17.3 Hz, 1H), 4.38 (d, J=17.3 Hz, 1H), 3.39-3.32 (m, 2H), 3.09 (m, 2H), 2.98-2.84 (m, 1H), 2.63-2.50 (m, 1H), 2.41 (m, 2H), 2.29-2.10 (m, 2H), 2.07-1.94 (m, 1H), 1.94-1.82 (d, J=13.6 Hz, 2H).

Step 3: To a solution of 4-(trifluoromethyl)benzaldehyde (13.42 mg, 0.077 mmol) and(S)-3-(4,6-difluoro-1-oxo-5-(piperidin-4-yl) isoindolin-2-yl) piperidine-2,6-dione, benzenesulfonic acid salt (20 mg, 0.038 mmol) dissolved in 1 mL DMF was added two drops of AcOH, and then stirred at room temperature. After 0.5 h, NaBH(OAc)₃ (24.50 mg, 0.116 mmol) was added and the resulting solution stirred at room temperature for 16 h. It was purified by Preparative HPLC Method 1 to obtain 7.2 mg of the titled compound. LC/MS (ESI) m/z 522.1 [(M+H)⁺, calcd for C₂₆H₂₄F₅N₃O₃ 521.2]; HPLC^{a} T_{Ret}=1.30 min; ¹H NMR (500 MHZ, DMSO-d₆) δ 11.01 (s, 1H), 7.65 (br d, J=7.6 Hz, 2H), 7.53 (br d, J=7.9 Hz, 2H), 7.40 (br d, J=8.5 Hz, 1H), 5.10-5.00 (m, 1H), 4.54-4.45 (m, 1H), 4.37-4.28 (m, 1H), 3.63-3.48 (m, 1H), 3.01-2.80 (m, 4H), 2.63-2.54 (m, 1H), 2.46 (br s, 4H), 2.41-2.29 (m, 1H), 2.09-1.89 (m, 5H), 1.70-1.60 (m, 2H).

### Example S4. 3-(5-(((4-(difluoromethyl)benzyl)amino)methyl)-4,6-difluoro-1-oxoisoindolin-2-yl) piperidine-2,6-dione (Compound 79)

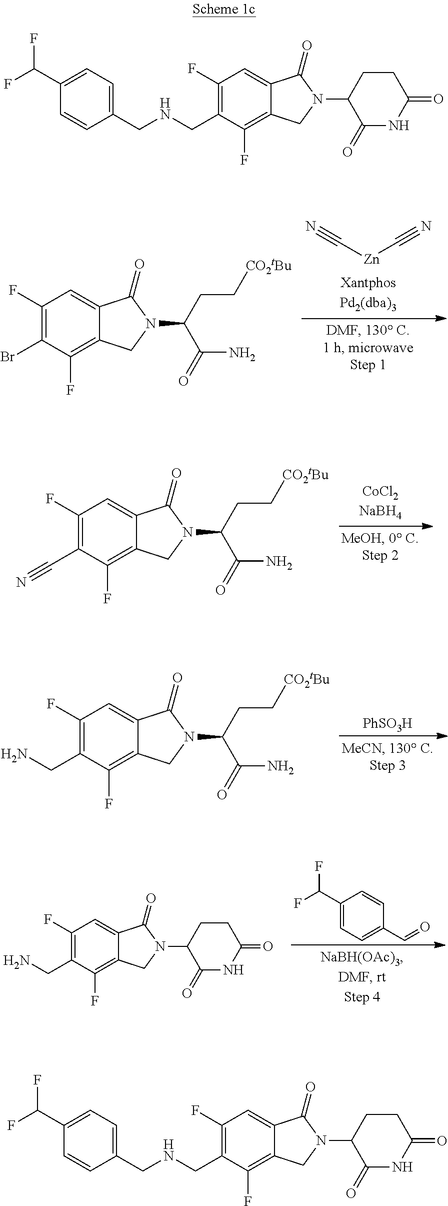

Step 1: To a microwave vial charged with tert-butyl(S)-5-amino-4-(5-bromo-4,6-difluoro-1-oxoisoindolin-2-yl)-5-oxopentanoate (1.00 g, 2.308 mmol), dicyanozinc (0.271 g, 2.308 mmol), Xantphos (0.033 g, 0.057 mmol), and Pd₂(dba)₃ (0.106 g, 0.115 mmol) was added DMF (20 mL). The air was replaced with argon and heated in the microwave at 130° C. for 1 h. It was diluted with EtOAc, washed with aq. 10% LiCl, and brine, dried over MgSO₄, and then concentrated. The crude product was purified by flash column chromatography eluting with 5-80% EtOAc/hexanes to obtain 501 mg (57% yield) of the desired product.

LC/MS (ESI) m/z 324.2 [(M-55)⁺, calcd for C₁₈H₁₉F₂N₃O₄ 379.1

Step 2: To a solution of tert-butyl(S)-5-amino-4-(5-cyano-4,6-difluoro-1-oxoisoindolin-2-yl)-5-oxopentanoate (200 mg, 0.527 mmol) in methanol (20 mL) at 0° C. was added cobalt (II) chloride (137 mg, 1.054 mmol) followed by NaBH₄ (19.94 mg, 0.527 mmol) and the air replaced with N₂. After 10 min the cooling bath was removed, and the reaction allowed to warm to room temperature. After 2 h, it was concentrated to dryness, and the residue suspended in EtOAc, washed with 1.5M aq. KH₂PO₄ solution. The organic layer was separated, and washed with brine, dried over Na₂SO₄, filtered and concentrated. The crude product was purified by flash column chromatography eluting with 0-50% B/DCM [where B=15% EtOH in EtOAc+0.1% TEA], to obtain tert-butyl 5-amino-4-(5-(aminomethyl)-4,6-difluoro-1-oxoisoindolin-2-yl)-5-oxopentanoate (150 mg, 74.2% yield).

LC/MS (ESI) m/z 384.2 [(M+H)⁺, calcd for C₁₈H₂₃F₂N₃O₄ 383.2

Step 3: A 5 mL microwave vial was charged with tert-butyl(S)-5-amino-4-(5-(aminomethyl)-4,6-difluoro-1-oxoisoindolin-2-yl)-5-oxopentanoate (149 mg, 0.389 mmol), benzenesulfonic acid (123 mg, 0.777 mmol) and MeCN (3 mL). It was heated to 130° C. for 0.5 h. It was concentrated to dryness, and the residues rinsed with ether to obtain a mono-PhSO₃H salt of the desired product (151 mg, 86% yield).

LC/MS (ESI) m/z 310.2 [(M+H)⁺, calcd for C₁₄H₁₃F₂N₃O₃ 309.3

Step 4: The titled compound was obtained in 18% yield, by reacting 3-(5-(aminomethyl)-4,6-difluoro-1-oxoisoindolin-2-yl) piperidine-2,6-dione with 4-(difluoromethyl)benzaldehyde via a reductive amination, by following the procedure outlined for the synthesis of Example S1.

LC/MS (ESI) m/z 450.0 [(M+H)⁺, calcd for C₂₂H₁₉F₄N₃O₃ 449.1]; HPLC^{a} T_{Ret}=1.11 min; ¹H NMR (500 MHz, DMSO-d₆) δ 11.05-10.95 (m, 1H), 7.55-7.46 (m, 5H), 5.12 (dd, J=13.2, 5.2 Hz, 1H), 4.58-4.50 (m, 1H), 4.39 (br d, J=17.6 Hz, 1H), 3.91-3.79 (m, 4H), 2.97-2.87 (m, 1H), 2.66-2.57 (m, 1H), 2.49-2.39 (m, 1H), 2.08-1.98 (m, 1H)

Examples of compounds prepared by following the procedures outlined for Scheme 1 and Schemes 1a-1c, using the appropriate aldehydes are listed in Table 2.

TABLE 2

| Compound | Example | | |
|---|---|---|---|
| No | SNo. | Structure | Chemical name |
| 13 | 5 | 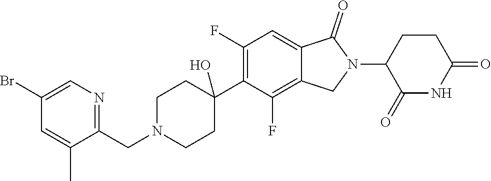 [SMILES: Cc1cc(Br)cnc1CN1CCC(O)(c2c(F)cc3c(c2F)CN(C2CCC(=O)NC2=O)C3=O)CC1] | 3-(5-(1-((5-bromo-3- methylpyridin-2-yl)methyl)-4- hydroxypiperidin-4-yl)-4,6- difluoro-1-oxoisoindolin-2- yl)piperidine-2,6-dione |
| 15 | 6 | 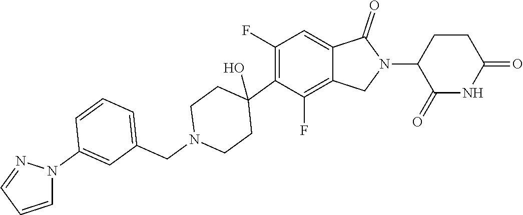 [SMILES: O=C1CCC(N2Cc3c(cc(F)c(C4(O)CCN(Cc5cccc(-n6cccn6)c5)CC4)c3F)C2=O)C(=O)N1] | 3-(5-(1-(3-(1H-pyrazol-1- yl)benzyl)-4- hydroxypiperidin-4-yl)-4,6- difluoro-1-oxoisoindolin-2- yl)piperidine-2,6-dione |
| 19 | 7 | 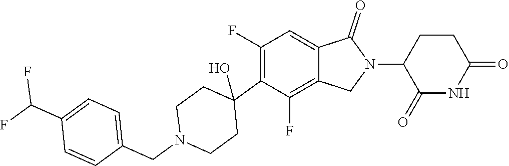 [SMILES: O=C1CCC(N2Cc3c(cc(F)c(C4(O)CCN(Cc5ccc(C(F)F)cc5)CC4)c3F)C2=O)C(=O)N1] | 3-(5-(1-(4- (difluoromethyl)benzyl)-4- hydroxypiperidin-4-yl)-4,6- difluoro-1-oxoisoindolin-2- yl)piperidine-2,6-dione |
| 20 | 8 | 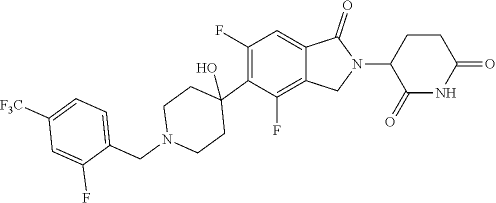 [SMILES: O=C1CCC(N2Cc3c(cc(F)c(C4(O)CCN(Cc5ccc(C(F)(F)F)cc5F)CC4)c3F)C2=O)C(=O)N1] | 3-(4,6-difluoro-5-(1-(2-fluoro- 4-(trifluoromethyl)benzyl)-4- hydroxypiperidin-4-yl)-1- oxoisoindolin-2-yl)piperidine- 2,6-dione |
| 21 | 9 | 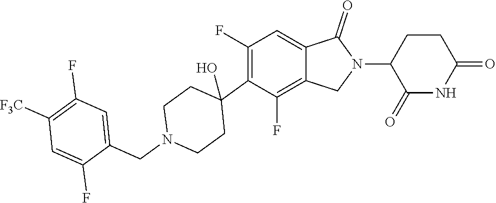 [SMILES: O=C1CCC(N2Cc3c(cc(F)c(C4(O)CCN(Cc5cc(F)c(C(F)(F)F)cc5F)CC4)c3F)C2=O)C(=O)N1] | 3-(5-(1-(2,5-difluoro-4- (trifluoromethyl)benzyl)-4- hydroxypiperidin-4-yl)-4,6- difluoro-1-oxoisoindolin-2- yl)piperidine-2,6-dione |
| 22 | 10 | 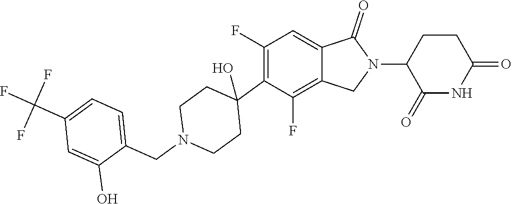 [SMILES: O=C1CCC(N2Cc3c(cc(F)c(C4(O)CCN(Cc5ccc(C(F)(F)F)cc5O)CC4)c3F)C2=O)C(=O)N1] | 3-(4,6-difluoro-5-(4-hydroxy- 1-(2-hydroxy-4- (trifluoromethyl)benzyl) piperidin- 4-yl)-1-oxoisoindolin-2- yl)piperidine-2,6-dione |
| 23 | 11 | 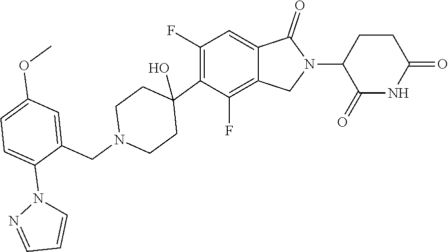 [SMILES: COc1ccc(-n2cccn2)c(CN2CCC(O)(c3c(F)cc4c(c3F)CN(C3CCC(=O)NC3=O)C4=O)CC2)c1] | 3-(4,6-difluoro-5-(4-hydroxy- 1-(5-methoxy-2-(1H-pyrazol- 1-yl)benzyl)piperidin-4-yl)-1- oxoisoindolin-2-yl)piperidine- 2,6-dione |
| 24 | 13 | 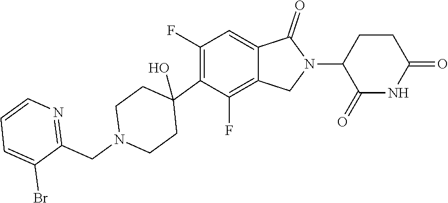 [SMILES: O=C1CCC(N2Cc3c(cc(F)c(C4(O)CCN(Cc5ncccc5Br)CC4)c3F)C2=O)C(=O)N1] | 3-(5-(1-((3-bromopyridin-2- yl)methyl)-4- hydroxypiperidin-4-yl)-4,6- difluoro-1-oxoisoindolin-2- yl)piperidine-2,6-dione |
| 25 | 14 | 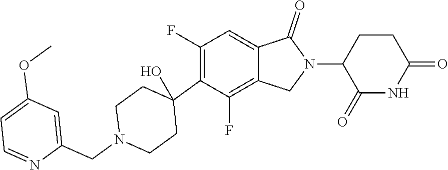 [SMILES: COc1ccnc(CN2CCC(O)(c3c(F)cc4c(c3F)CN(C3CCC(=O)NC3=O)C4=O)CC2)c1] | 3-(4,6-difluoro-5-(4-hydroxy- 1-((4-methoxypyridin-2- yl)methyl)piperidin-4-yl)-1- oxoisoindolin-2-yl)piperidine- 2,6-dione |
| 26 | 15 | 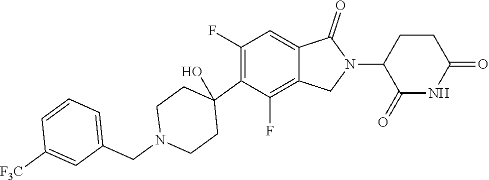 [SMILES: O=C1CCC(N2Cc3c(cc(F)c(C4(O)CCN(Cc5cccc(C(F)(F)F)c5)CC4)c3F)C2=O)C(=O)N1] | 3-(4,6-difluoro-5-(4-hydroxy- 1-(3- (trifluoromethyl)benzyl) piperidin- 4-yl)-1-oxoisoindolin-2- yl)piperidine-2,6-dione |
| 32 | 16 | 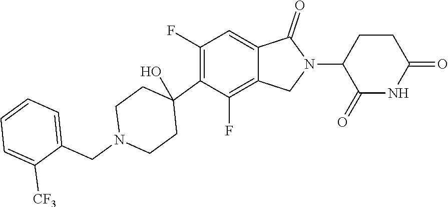 [SMILES: O=C1CCC(N2Cc3c(cc(F)c(C4(O)CCN(Cc5ccccc5C(F)(F)F)CC4)c3F)C2=O)C(=O)N1] | 3-(4,6-difluoro-5-(4-hydroxy- 1-(2- (trifluoromethyl)benzyl) piperidin- 4-yl)-1-oxoisoindolin-2- yl)piperidine-2,6-dione |
| 47 | 17 | 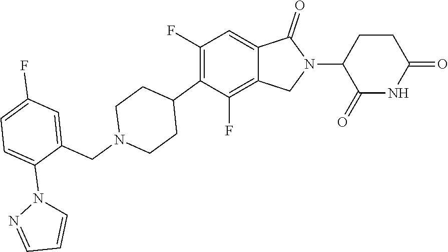 [SMILES: O=C1CCC(N2Cc3c(cc(F)c(C4CCN(Cc5cc(F)ccc5-n5cccn5)CC4)c3F)C2=O)C(=O)N1] | 3-(4,6-difluoro-5-(1-(5-fluoro- 2-(1H-pyrazol-1- yl)benzyl)piperidin-4-yl)-1- oxoisoindolin-2-yl)piperidine- 2,6-dione |
| 48 | 18 | 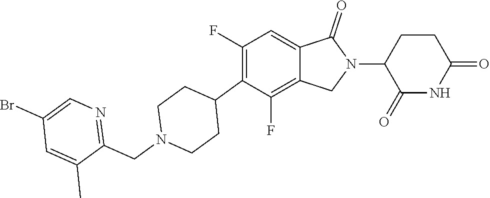 [SMILES: Cc1cc(Br)cnc1CN1CCC(c2c(F)cc3c(c2F)CN(C2CCC(=O)NC2=O)C3=O)CC1] | 3-(5-(1-((5-bromo-3- methylpyridin-2- yl)methyl)piperidin-4-yl)-4,6- difluoro-1-oxoisoindolin-2- yl)piperidine-2,6-dione |
| 49 | 19 | 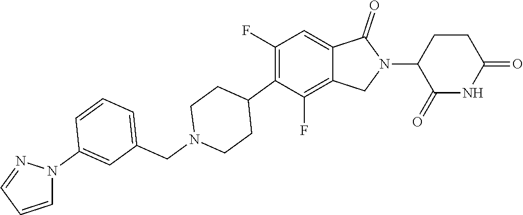 [SMILES: O=C1CCC(N2Cc3c(cc(F)c(C4CCN(Cc5cccc(-n6cccn6)c5)CC4)c3F)C2=O)C(=O)N1] | 3-(5-(1-(3-(1H-pyrazol-1- yl)benzyl)piperidin-4-yl)-4,6- difluoro-1-oxoisoindolin-2- yl)piperidine-2,6-dione |
| 50 | 20 | 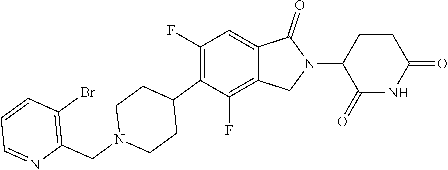 [SMILES: O=C1CCC(N2Cc3c(cc(F)c(C4CCN(Cc5ncccc5Br)CC4)c3F)C2=O)C(=O)N1] | 3-(5-(1-((3-bromopyridin-2- yl)methyl)piperidin-4-yl)-4,6- difluoro-1-oxoisoindolin-2- yl)piperidine-2,6-dione |
| 51 | 21 | 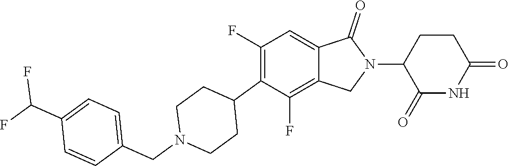 [SMILES: O=C1CCC(N2Cc3c(cc(F)c(C4CCN(Cc5ccc(C(F)F)cc5)CC4)c3F)C2=O)C(=O)N1] | 3-(5-(1-(4- (difluoromethyl)benzyl) piperidin- 4-yl)-4,6-difluoro-1- oxoisoindolin-2-yl)piperidine- 2,6-dione |
| 52 | 2 | 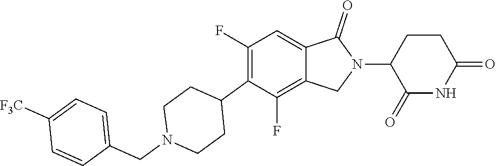 [SMILES: O=C1CCC(N2Cc3c(cc(F)c(C4CCN(Cc5ccc(C(F)(F)F)cc5)CC4)c3F)C2=O)C(=O)N1] | 3-(4,6-difluoro-1-oxo-5-(1-(4- (trifluoromethyl)benzyl) piperidin- 4-yl)isoindolin-2- yl)piperidine-2,6-dione |
| 53 | 22 | 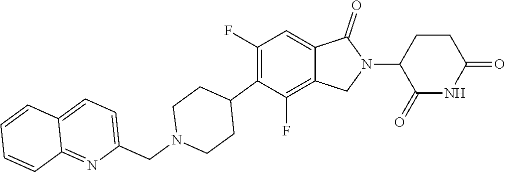 [SMILES: O=C1CCC(N2Cc3c(cc(F)c(C4CCN(Cc5ccc6ccccc6n5)CC4)c3F)C2=O)C(=O)N1] | 3-(4,6-difluoro-1-oxo-5-(1- (quinolin-2- ylmethyl)piperidin-4- yl)isoindolin-2-yl)piperidine- 2,6-dione |
| 54 | 23 | 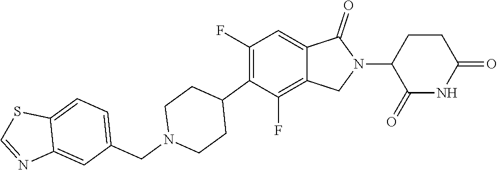 [SMILES: O=C1CCC(N2Cc3c(cc(F)c(C4CCN(Cc5ccc6scnc6c5)CC4)c3F)C2=O)C(=O)N1] | 3-(5-(1-(benzo[d]thiazol-5- ylmethyl)piperidin-4-yl)-4,6- difluoro-1-oxoisoindolin-2- yl)piperidine-2,6-dione |
| 55 | 24 | 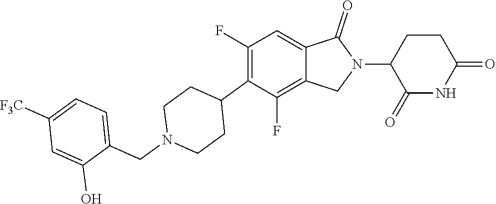 [SMILES: O=C1CCC(N2Cc3c(cc(F)c(C4CCN(Cc5ccc(C(F)(F)F)cc5O)CC4)c3F)C2=O)C(=O)N1] | 3-(4,6-difluoro-5-(1-(2- hydroxy-4- (trifluoromethyl)benzyl) piperidin- 4-yl)-1-oxoisoindolin-2- yl)piperidine-2,6-dione |
| 56 | 25 | 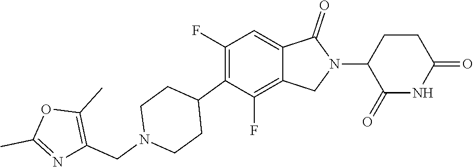 [SMILES: Cc1nc(CN2CCC(c3c(F)cc4c(c3F)CN(C3CCC(=O)NC3=O)C4=O)CC2)c(C)o1] | 3-(5-(1-((2,5-dimethyloxazol- 4-yl)methyl)piperidin-4-yl)- 4,6-difluoro-1-oxoisoindolin- 2-yl)piperidine-2,6-dione |
| 60 | 27 | 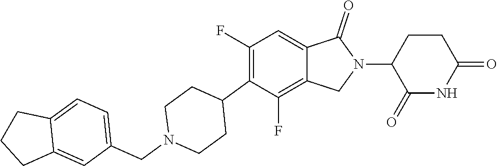 [SMILES: O=C1CCC(N2Cc3c(cc(F)c(C4CCN(Cc5ccc6c(c5)CCC6)CC4)c3F)C2=O)C(=O)N1] | 3-(5-(1-((2,3-dihydro-1H- inden-5-yl)methyl)piperidin- 4-yl)-4,6-difluoro-1- oxoisoindolin-2-yl)piperidine- 2,6-dione |
| 61 | 28 | 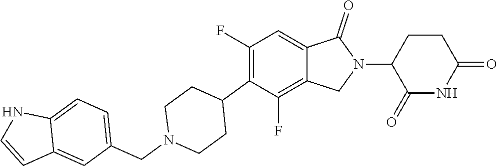 [SMILES: O=C1CCC(N2Cc3c(cc(F)c(C4CCN(Cc5ccc6[nH]ccc6c5)CC4)c3F)C2=O)C(=O)N1] | 3-(5-(1-((1H-indol-5- yl)methyl)piperidin-4-yl)-4,6- difluoro-1-oxoisoindolin-2- yl)piperidine-2,6-dione |
| 62 | 29 | 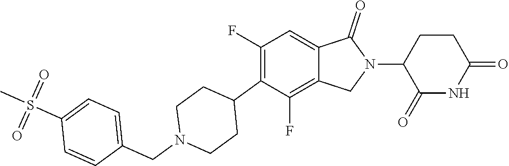 [SMILES: CS(=O)(=O)c1ccc(CN2CCC(c3c(F)cc4c(c3F)CN(C3CCC(=O)NC3=O)C4=O)CC2)cc1] | 3-(4,6-difluoro-5-(1-(4- (methylsulfonyl)benzyl) piperidin- 4-yl)-1-oxoisoindolin-2- yl)piperidine-2,6-dione |
| 63 | 30 | 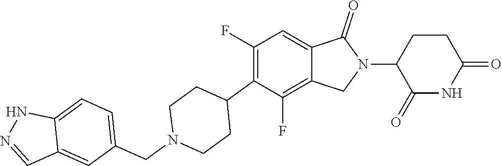 [SMILES: O=C1CCC(N2Cc3c(cc(F)c(C4CCN(Cc5ccc6[nH]ncc6c5)CC4)c3F)C2=O)C(=O)N1] | 3-(5-(1-((1H-indazol-5- yl)methyl)piperidin-4-yl)-4,6- difluoro-1-oxoisoindolin-2- yl)piperidine-2,6-dione |
| 64 | 31 | 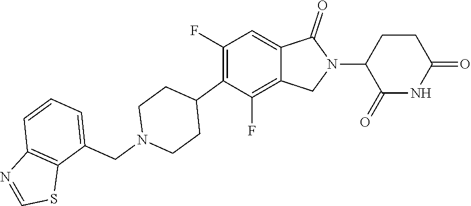 [SMILES: O=C1CCC(N2Cc3c(cc(F)c(C4CCN(Cc5cccc6ncsc56)CC4)c3F)C2=O)C(=O)N1] | 3-(5-(1-(benzo[d]thiazol-7- ylmethyl)piperidin-4-yl)-4,6- difluoro-1-oxoisoindolin-2- yl)piperidine-2,6-dione |
| 68 | 33 | 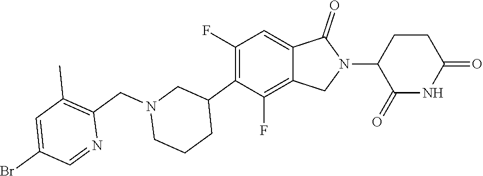 [SMILES: Cc1cc(Br)cnc1CN1CCCC(c2c(F)cc3c(c2F)CN(C2CCC(=O)NC2=O)C3=O)C1] | 3-(5-(1-((5-bromo-3- methylpyridin-2- yl)methyl)piperidin-3-yl)-4,6- difluoro-1-oxoisoindolin-2- yl)piperidine-2,6-dione |
| 37 | 35 | 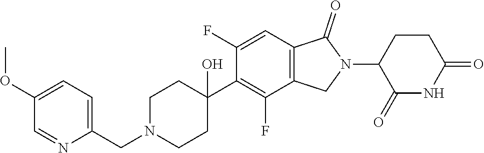 [SMILES: COc1ccc(CN2CCC(O)(c3c(F)cc4c(c3F)CN(C3CCC(=O)NC3=O)C4=O)CC2)nc1] | 3-(4,6-difluoro-5-(4-hydroxy- 1-((5-methoxypyridin-2- yl)methyl)piperidin-4-yl)-1- oxoisoindolin-2-yl)piperidine- 2,6-dione |
| 41 | 38 | 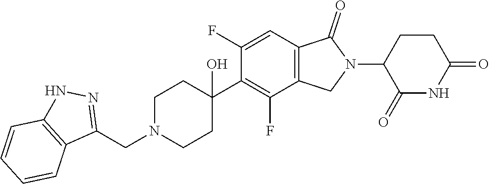 [SMILES: O=C1CCC(N2Cc3c(cc(F)c(C4(O)CCN(Cc5n[nH]c6ccccc56)CC4)c3F)C2=O)C(=O)N1] | 3-[4,6-difluoro-5-[4-hydroxy- 1-(1H-indazol-3-ylmethyl)-4- piperidyl]-1-oxo-isoindolin-2- yl] piperidine-2,6-dione |
| 42 | 40 | 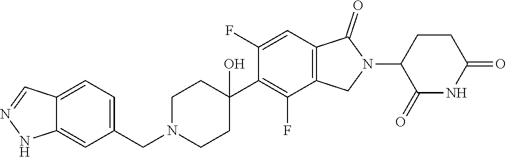 [SMILES: O=C1CCC(N2Cc3c(cc(F)c(C4(O)CCN(Cc5ccc6cn[nH]c6c5)CC4)c3F)C2=O)C(=O)N1] | 3-[4,6-difluoro-5-[4-hydroxy- 1-(1H-indazol-6-ylmethyl)-4- piperidyl]-1-oxo-isoindolin-2- yl]piperidine-2,6-dione |
| 43 | 41 | 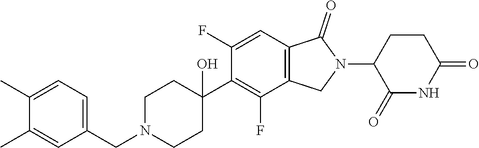 [SMILES: Cc1ccc(CN2CCC(O)(c3c(F)cc4c(c3F)CN(C3CCC(=O)NC3=O)C4=O)CC2)cc1C] | 3-[5-[1-[(3,4- dimethylphenyl)methyl]-4- hydroxy-4-piperidyl]-4,6- difluoro-1-oxo-isoindolin-2- yl]piperidine-2,6-dione |
| 44 | 42 | 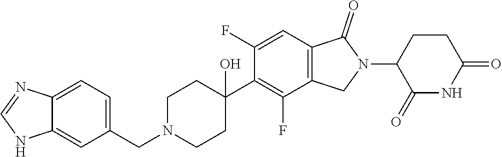 [SMILES: O=C1CCC(N2Cc3c(cc(F)c(C4(O)CCN(Cc5ccc6nc[nH]c6c5)CC4)c3F)C2=O)C(=O)N1] | 3-[5-[1-(3H-benzimidazol-5- ylmethyl)-4-hydroxy-4- piperidyl]-4,6-difluoro-1-oxo- isoindolin-2-yl]piperidine- 2,6-dione |
| 37 | 43 | 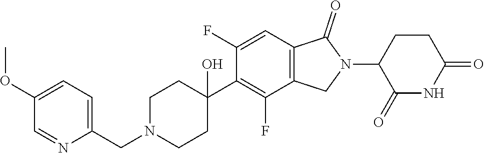 [SMILES: COc1ccc(CN2CCC(O)(c3c(F)cc4c(c3F)CN(C3CCC(=O)NC3=O)C4=O)CC2)nc1] | 3-[4,6-difluoro-5-[4-hydroxy- 1-[(5-methoxy-2- pyridyl)methyl]-4-piperidyl]- 1-oxo-isoindolin-2- yl]piperidine-2,6-dione |
| 39 | 46 | 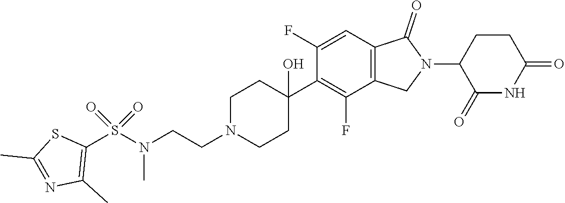 [SMILES: Cc1nc(C)c(S(=O)(=O)N(C)CCN2CCC(O)(c3c(F)cc4c(c3F)CN(C3CCC(=O)NC3=O)C4=O)CC2)s1] | N-[2-[4-[2-(2,6-dioxo-3- piperidyl)-4,6-difluoro-1-oxo- isoindolin-5-yl]-4-hydroxy-1- piperidyl]ethyl]-N,2,4- trimethyl-thiazole-5- sulfonamide |
| 40 | 47 | 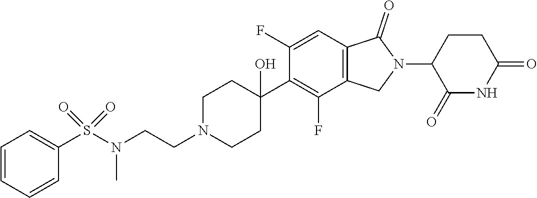 [SMILES: CN(CCN1CCC(O)(c2c(F)cc3c(c2F)CN(C2CCC(=O)NC2=O)C3=O)CC1)S(=O)(=O)c1ccccc1] | N-[2-[4-[2-(2,6-dioxo-3- piperidyl)-4,6-difluoro-1-oxo- isoindolin-5-yl]-4-hydroxy-1- piperidyl]ethyl]-N-methyl- benzenesulfonamide |
| 70 | 55 | 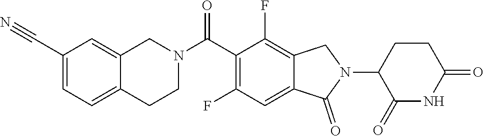 [SMILES: N#Cc1ccc2c(c1)CN(C(=O)c1c(F)cc3c(c1F)CN(C1CCC(=O)NC1=O)C3=O)CC2] | 2-[2-(2,6-dioxo-3-piperidyl)- 4,6-difluoro-1-oxo- isoindoline-5-carbonyl]-3,4- dihydro-1H-isoquinoline-7- carbonitrile |
| 71 | 56 | 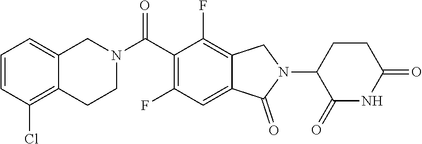 [SMILES: O=C1CCC(N2Cc3c(cc(F)c(C(=O)N4CCc5c(Cl)cccc5C4)c3F)C2=O)C(=O)N1] | 3-[5-(5-chloro-3,4-dihydro- 1H-isoquinoline-2-carbonyl)- 4,6-difluoro-1-oxo-isoindolin- 2-yl]piperidine-2,6-dione |
| 72 | 57 | 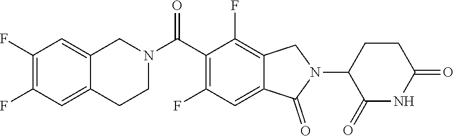 [SMILES: O=C1CCC(N2Cc3c(cc(F)c(C(=O)N4CCc5cc(F)c(F)cc5C4)c3F)C2=O)C(=O)N1] | 3-[5-(6, 7-difluoro-3,4- dihydro-1H-isoquinoline-2- carbonyl)-4,6-difluoro-1-oxo- isoindolin-2-yl] piperidine- 2,6-dione |
| 73 | 58 | 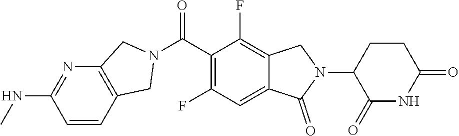 [SMILES: CNc1ccc2c(n1)CN(C(=O)c1c(F)cc3c(c1F)CN(C1CCC(=O)NC1=O)C3=O)C2] | 3-[4,6-difluoro-5-[2- (methylamino)-5,7- dihydropyrrolo[3,4-b] pyridine-6-carbonyl]-1-oxo- isoindolin-2-yl] piperidine- 2,6-dione |
| 69 | 66 | 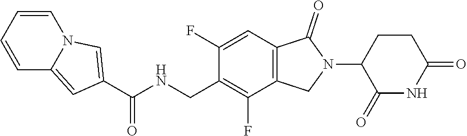 [SMILES: O=C1CCC(N2Cc3c(cc(F)c(CNC(=O)c4cc5ccccn5c4)c3F)C2=O)C(=O)N1] | N-[[2-(2,6-dioxo-3-piperidyl)- 4,6-difluoro-1-oxo-isoindolin- 5-yl]methyl]indolizine-2- carboxamide |

### Example S68. 3-(5-(1-(benzo[d]thiazol-5-ylmethyl)-4-hydroxypiperidin-4-yl)-4,6-difluoro-1-oxoisoindolin-2-yl) piperidine-2,6-dione (Compound 12)

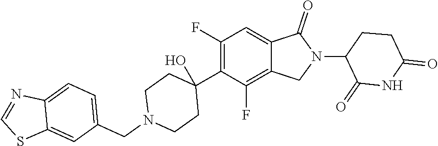 [SMILES: O=C1CCC(N2Cc3c(cc(F)c(C4(O)CCN(Cc5ccc6ncsc6c5)CC4)c3F)C2=O)C(=O)N1]

To 6-(chloromethyl)benzo[d]thiazole (7.5 mg, 0.041 mmol) was added 3-(4,6-difluoro-5-(4-hydroxypiperidin-4-yl)-1-oxoisoindolin-2-yl) piperidine-2,6-dione (14 mg, 0.037 mmol) dissolved in 1 mL DMF followed by the addition of Hunig's base (0.045 mL, 0.258 mmol). The resulting mixture was heated at 85° C. for 2 h. It was cooled to room temperature, and purified by Preparative HPLC Method 1 to obtain 6.0 mg of the titled compound.

LC/MS (ESI) m/z 527.0 [(M+H)⁺, calcd for C₂₆H₂₄F₂N₄O₄S 526.1]; HPLC^{a} T_{Ret}=0.93 min; ¹H NMR (500 MHz, DMSO-d₆) δ 10.96 (s, 1H), 9.34 (s, 1H), 8.16-8.09 (m, 1H), 8.04 (br d, J=8.9 Hz, 1H), 7.59-7.49 (m, 1H), 7.36 (br d, J=10.4 Hz, 1H), 5.62-5.40 (m, 1H), 5.05 (br dd, J=13.3, 5.0 Hz, 1H), 4.47 (br d, J=17.4 Hz, 1H), 4.30 (br d, J=17.4 Hz, 1H), 3.52-3.30 (m, 1H), 2.90-2.81 (m, 1H), 2.59-2.53 (m, 1H), 2.46 (br s, 5H), 2.43-2.32 (m, 1H), 2.22 (br s, 1H), 1.99-1.93 (m, 2H).

### Example S69. 3-(5-(1-(benzo[d]thiazol-6-ylmethyl) piperidin-4-yl)-4,6-difluoro-1-oxoisoindolin-2-yl) piperidine-2,6-dione (Compound 18)

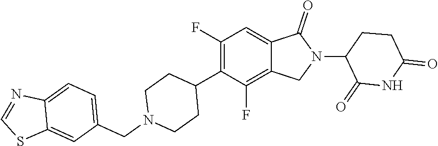 [SMILES: O=C1CCC(N2Cc3c(cc(F)c(C4CCN(Cc5ccc6ncsc6c5)CC4)c3F)C2=O)C(=O)N1]

The titled compound was obtained in 34% yield as an off-white solid by following the alkylation procedure outlined for the synthesis of Example S68 using 3-(4,6-difluoro-1-oxo-5-(piperidin-4-yl) isoindolin-2-yl) piperidine-2,6-dione as the amine coupling partner.

LC/MS (ESI) m/z 511.1 [(M+H)⁺, calcd for C₂₆H₂₄F₂N₄O₃S 510.2]; HPLC^{a} T_{Ret}=0.93 min; ¹H NMR (500 MHZ, DMSO-d₆) δ 10.96 (s, 1H), 9.47 (s, 1H), 8.29 (s, 1H), 8.16 (d, J=8.2 Hz, 1H), 7.66 (br d, J=8.5 Hz, 1H), 7.44 (br d, J=8.9 Hz, 1H), 5.06 (br dd, J=13.6, 5.0 Hz, 1H), 4.53-4.41 (m, 2H), 4.33 (br d, J=17.1 Hz, 1H), 2.92-2.80 (m, 1H), 2.56 (br d, J=17.1 Hz, 1H), 2.46 (br s, 5H), 2.43-2.31 (m, 1H), 2.29-2.16 (m, 1H), 1.99-1.89 (m, 2H).

### Example S71. 3-(5-(1-(5-bromo-3-methylpicolinoyl) piperidin-4-yl)-4,6-difluoro-1-oxoisoindolin-2-yl) piperidine-2,6-dione (Compound 66)

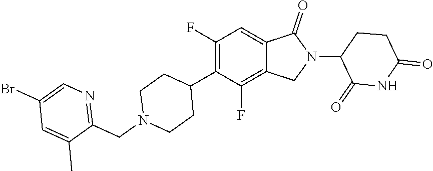 [SMILES: Cc1cc(Br)cnc1CN1CCC(c2c(F)cc3c(c2F)CN(C2CCC(=O)NC2=O)C3=O)CC1]

The titled compound was obtained in 39% yield as a white solid by following the amidation procedure outlined in General Scheme 1 using 3-(4,6-difluoro-1-oxo-5-(piperidin-4-yl) isoindolin-2-yl) piperidine-2,6-dione as the amine coupling partner.

LC/MS (ESI) m/z 561.0 [(M+H)⁺, calcd for C₂₅H₂₃BrF₂N₄O₄ 560.1]; HPLC^{a} T_{Ret}=1.52 min; ¹H NMR (500 MHz, DMSO-d₆) δ 10.96 (s, 1H), 8.51 (s, 1H), 8.02 (s, 1H), 7.42 (d, J=8.7 Hz, 1H), 5.05 (dd, J=13.3, 4.9 Hz, 1H), 4.63 (br d, J=12.6 Hz, 1H), 4.50 (d, J=17.3 Hz, 1H), 4.33 (d, J=17.1 Hz, 1H), 3.18-3.12 (m, 1H), 2.93-2.80 (m, 2H), 2.56 (br d, J=17.5 Hz, 1H), 2.46 (s, 4H), 2.43-2.33 (m, 1H), 2.23 (s, 3H), 2.01-1.79 (m, 3H), 1.63 (br d, J=12.8 Hz, ¹H

1.76 (m, 3H)

Examples of compounds prepared by following the alkylation procedures outlined for the syntheses of Example S68, Example S69, and Example S72, using the appropriate the alkyl halides, and also the amidation procedures outlined for the syntheses of Example S70 and Example S71 using the appropriate carboxylic acids are listed in Table 3.

TABLE 3

| Cmpd | Example | | |
|---|---|---|---|
| No | SNo. | Structure | Chemical name |
| 14 | 73 | 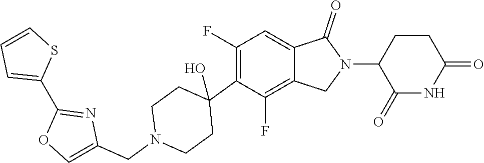 [SMILES: O=C1CCC(N2Cc3c(cc(F)c(C4(O)CCN(Cc5coc(-c6cccs6)n5)CC4)c3F)C2=O)C(=O)N1] | 3-(4,6-difluoro-5-(4-hydroxy- 1-((2-(thiophen-2-yl)oxazol-4- yl)methyl)piperidin-4-yl)-1- oxoisoindolin-2-yl)piperidine- 2,6-dione |
| 17 | 74 | 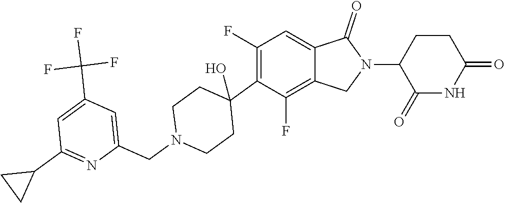 [SMILES: O=C1CCC(N2Cc3c(cc(F)c(C4(O)CCN(Cc5cc(C(F)(F)F)cc(C6CC6)n5)CC4)c3F)C2=O)C(=O)N1] | 3-(5-(1-((6-cyclopropyl-4- (trifluoromethyl)pyridin-2- yl)methyl)-4- hydroxypiperidin-4-yl)-4,6- difluoro-1-oxoisoindolin-2- yl)piperidine-2,6-dione |
| 27 | 75 | 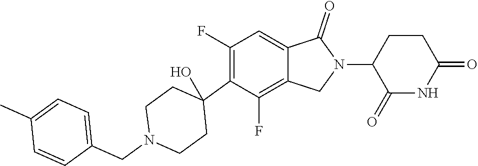 [SMILES: Cc1ccc(CN2CCC(O)(c3c(F)cc4c(c3F)CN(C3CCC(=O)NC3=O)C4=O)CC2)cc1] | 3-(4,6-difluoro-5-(4-hydroxy- 1-(4-methylbenzyl)piperidin-4- yl)-1-oxoisoindolin-2- yl)piperidine-2,6-dione |
| 28 | 76 | 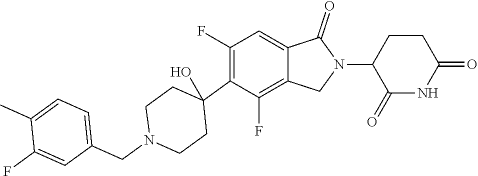 [SMILES: Cc1ccc(CN2CCC(O)(c3c(F)cc4c(c3F)CN(C3CCC(=O)NC3=O)C4=O)CC2)cc1F] | 3-(4,6-difluoro-5-(1-(3-fluoro- 4-methylbenzyl)-4- hydroxypiperidin-4-yl)-1- oxoisoindolin-2-yl)piperidine- 2,6-dione |
| 29 | 77 | 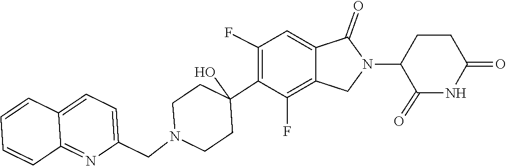 [SMILES: O=C1CCC(N2Cc3c(cc(F)c(C4(O)CCN(Cc5ccc6ccccc6n5)CC4)c3F)C2=O)C(=O)N1] | 3-(4,6-difluoro-5-(4-hydroxy- 1-(quinolin-2- ylmethyl)piperidin-4-yl)-1- oxoisoindolin-2-yl)piperidine- 2,6-dione |
| 30 | 78 | 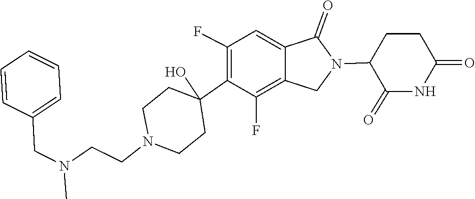 [SMILES: CN(CCN1CCC(O)(c2c(F)cc3c(c2F)CN(C2CCC(=O)NC2=O)C3=O)CC1)Cc1ccccc1] | 3-(5-(1-(2- (benzyl(methyl)amino)ethyl)- 4-hydroxypiperidin-4-yl)-4,6- difluoro-1-oxoisoindolin-2- yl)piperidine-2,6-dione |
| 31 | 79 | 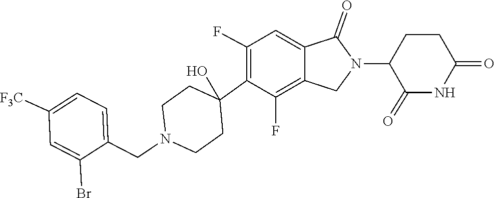 [SMILES: O=C1CCC(N2Cc3c(cc(F)c(C4(O)CCN(Cc5ccc(C(F)(F)F)cc5Br)CC4)c3F)C2=O)C(=O)N1] | 3-(5-(1-(2-bromo-4- (trifluoromethyl)benzyl)-4- hydroxypiperidin-4-yl)-4,6- difluoro-1-oxoisoindolin-2- yl)piperidine-2,6-dione |
| 33 | 80 | 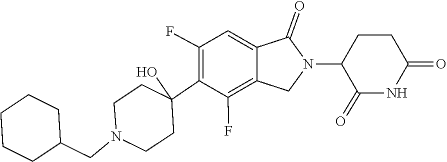 [SMILES: O=C1CCC(N2Cc3c(cc(F)c(C4(O)CCN(CC5CCCCC5)CC4)c3F)C2=O)C(=O)N1] | 3-(5-(1-(cyclohexylmethyl)-4- hydroxypiperidin-4-yl)-4,6- difluoro-1-oxoisoindolin-2- yl)piperidine-2,6-dione |
| 57 | 81 | 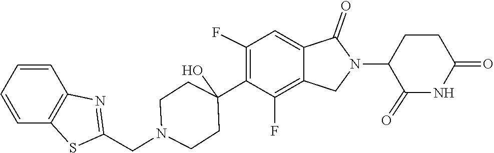 [SMILES: O=C1CCC(N2Cc3c(cc(F)c(C4(O)CCN(Cc5nc6ccccc6s5)CC4)c3F)C2=O)C(=O)N1] | 3-(5-(1-(benzo[d]thiazol-2- ylmethyl)-4-hydroxypiperidin- 4-yl)-4,6-difluoro-1- oxoisoindolin-2-yl)piperidine- 2,6-dione |
| 45 | 82 | 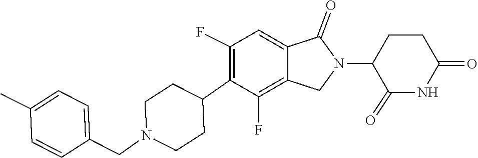 [SMILES: Cc1ccc(CN2CCC(c3c(F)cc4c(c3F)CN(C3CCC(=O)NC3=O)C4=O)CC2)cc1] | 3-(4,6-difluoro-5-(1-(4- methylbenzyl)piperidin-4-yl)- 1-oxoisoindolin-2- yl)piperidine-2,6-dione |
| 46 | 84 | 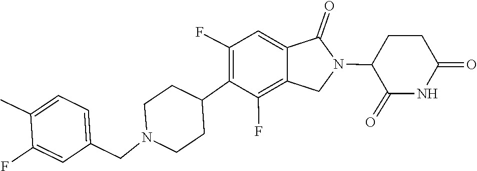 [SMILES: Cc1ccc(CN2CCC(c3c(F)cc4c(c3F)CN(C3CCC(=O)NC3=O)C4=O)CC2)cc1F] | 3-(4,6-difluoro-5-(1-(3-fluoro- 4-methylbenzyl)piperidin-4- yl)-1-oxoisoindolin-2- yl)piperidine-2,6-dione |
| 58 | 85 | 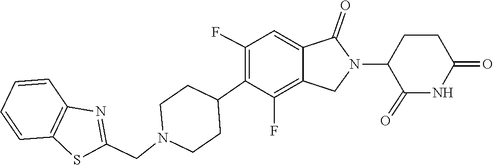 [SMILES: O=C1CCC(N2Cc3c(cc(F)c(C4CCN(Cc5nc6ccccc6s5)CC4)c3F)C2=O)C(=O)N1] | 3-(5-(1-(benzo[d]thiazol-2- ylmethyl)piperidin-4-yl)-4,6- difluoro-1-oxoisoindolin-2- yl)piperidine-2,6-dione |
| 59 | 86 | 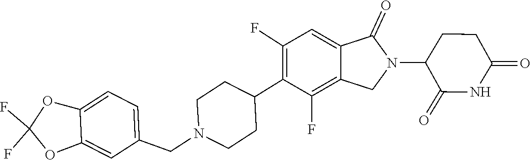 [SMILES: O=C1CCC(N2Cc3c(cc(F)c(C4CCN(Cc5ccc6c(c5)OC(F)(F)O6)CC4)c3F)C2=O)C(=O)N1] | 3-(5-(1-((2,2- difluorobenzo[d][1,3]dioxol-5- yl)methyl)piperidin-4-yl)-4,6- difluoro-1-oxoisoindolin-2- yl)piperidine-2,6-dione |
| 65 | 87 | 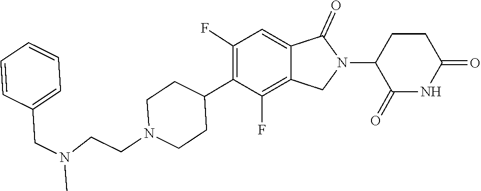 [SMILES: CN(CCN1CCC(c2c(F)cc3c(c2F)CN(C2CCC(=O)NC2=O)C3=O)CC1)Cc1ccccc1] | 3-(5-(1-(2- (benzyl(methyl)amino)ethyl)pi peridin-4-yl)-4,6-difluoro-1- oxoisoindolin-2-yl)piperidine- 2,6-dione |
| 67 | 88 | 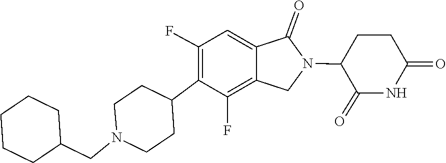 [SMILES: O=C1CCC(N2Cc3c(cc(F)c(C4CCN(CC5CCCCC5)CC4)c3F)C2=O)C(=O)N1] | 3-(5-(1- (cyclohexylmethyl)piperidin-4- yl)-4,6-difluoro-1- oxoisoindolin-2-yl)piperidine- 2,6-dione |
| 78 | 97 | 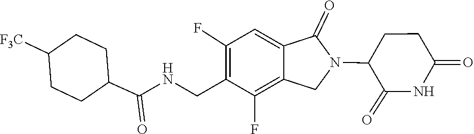 [SMILES: O=C1CCC(N2Cc3c(cc(F)c(CNC(=O)C4CCC(C(F)(F)F)CC4)c3F)C2=O)C(=O)N1] | N-((2-(2,6-dioxopiperidin-3- yl)-4,6-difluoro-1- oxoisoindolin-5-yl)methyl)-4- (trifluoromethyl)cyclohexane- 1-carboxamide |
| 92 | 98 | 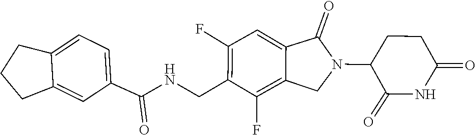 [SMILES: O=C1CCC(N2Cc3c(cc(F)c(CNC(=O)c4ccc5c(c4)CCC5)c3F)C2=O)C(=O)N1] | N-((2-(2,6-dioxopiperidin-3- yl)-4,6-difluoro-1- oxoisoindolin-5-yl)methyl)- 2,3-dihydro-1H-indene-5- carboxamide |

### Example S101. N-[2-[4-[2-(2,6-dioxo-3-piperidyl)-4,6-difluoro-1-oxo-isoindolin-5-yl]-4-hydroxy-1-piperidyl]ethyl]-N,2,4-trimethyl-thiazole-5-sulfonamide (Compound 39)

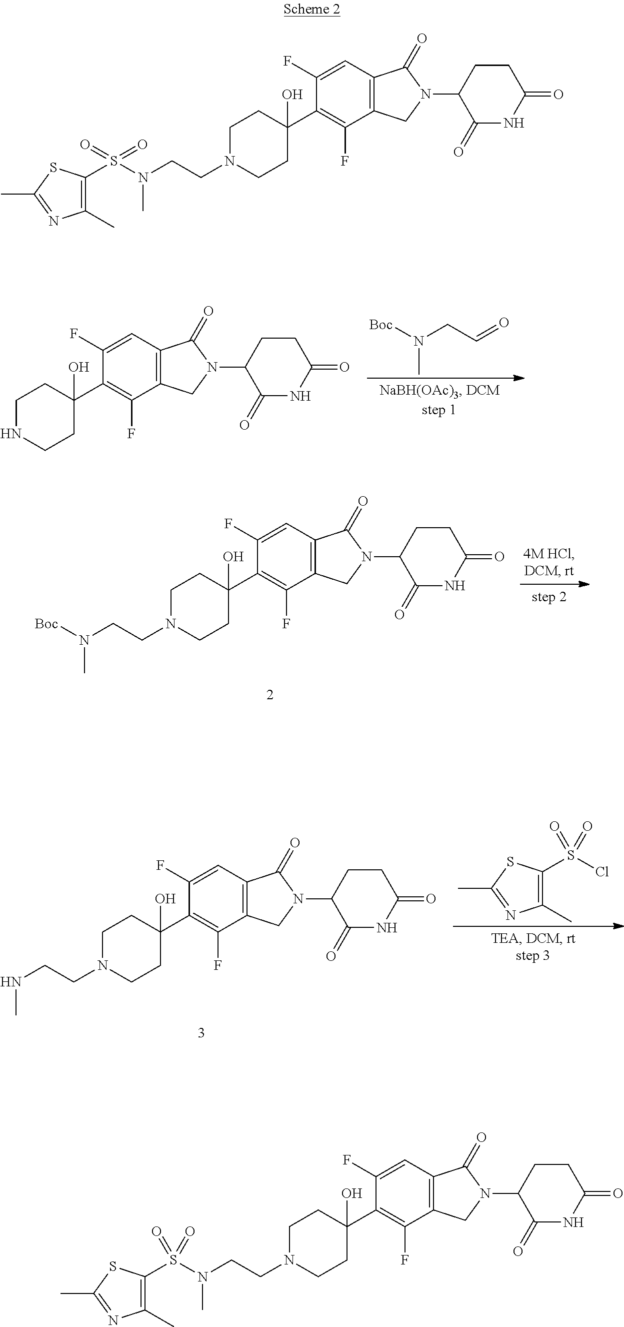

Step 1: To a solution of rac-(3S)-3-[4,6-difluoro-5-(4-hydroxy-4-piperidyl)-1-oxo-isoindolin-2-yl]piperidine-2,6-dione (150 mg, 0.4 mmol) in DCM (5 mL) was added tert-butyl N-methyl-N-(2-oxoethyl) carbamate (205.4 mg, 1.19 mmol) and NaBH(OAc)₃ (251.4 mg, 1.19 mmol). The resulting solution was stirred at room temperature for 2 h. The reaction was monitored by LCMS. The mixture was concentrated under reduced pressure. The product was purified reverse flash chromatography (column, C18 silica gel; mobile phase, ACN and water (0.05% TFA), 10% ACN to 70% ACN gradient in 20 min; detector, UV 254 nm) to afford tert-butyl N-[2-[4-[4,6-difluoro-1-oxo-2-[rac-(3S)-2,6-dioxo-3-piperidyl]isoindolin-5-yl]-4-hydroxy-1-piperidyl]ethyl]-N-methyl-carbamate (150 mg, 63.6% yield) as a light brown solid.

MS: m/z: Calc'd for C₂₆H₃₄F₂N₄O₆ [M+H]⁺ 537; Found 537.

Step 2: To a solution of tert-butyl N-[2-[4-[4,6-difluoro-1-oxo-2-[rac-(3S)-2,6-dioxo-3-piperidyl]isoindolin-5-yl]-4-hydroxy-1-piperidyl]ethyl]-N-methyl-carbamate (150 mg, 0.28 mmol) in DCM (4 mL) was added 4M HCl (1 mL) in 1,4-dioxane. The resulting mixture was stirred at room temperature for 3 h. The reaction was monitored by LCMS. The mixture was concentrated under reduced pressure to afford rac-(3S)-3-[4,6-difluoro-5-[4-hydroxy-1-[2-(methylamino)ethyl]-4-piperidyl]-1-oxo-isoindolin-2-yl]piperidine-2,6-dione (130 mg crude) as a brown solid.

MS: m/z: Calc'd for C₂₁H₂₆F₂N₄O₄ [M+H]⁺437; Found 437.

Step 3: To a solution of rac-(3S)-3-[4,6-difluoro-5-[4-hydroxy-1-[2-(methylamino)ethyl]-4-piperidyl]-1-oxo-isoindolin-2-yl]piperidine-2,6-dione (65 mg, 0.15 mmol) in DCM (2 mL) was added 2,4-dimethylthiazole-5-sulfonyl chloride (63.8 mg, 0.3 mmol) and TEA (58.4 mg, 0.45 mmol). The resulting solution was stirred at room temperature for 2 h. The reaction was monitored by LCMS. The mixture was concentrated under reduced pressure. The product was purified by Prep-HPLC to afford N-[2-[4-[2-(2,6-dioxo-3-piperidyl)-4,6-difluoro-1-oxo-isoindolin-5-yl]-4-hydroxy-1-piperidyl]ethyl]-N,2,4-trimethyl-thiazole-5-sulfonamide (34.7 mg, 37.4% yield) as a light brown solid.

MS: m/z: Calc'd for C₂₆H₃₁F₂N₅O₆S₂ [M+H]⁺610; Found 610. ¹H NMR (300 MHZ, DMSO-d₆) δ 11.03 (s, 1H), 9.47 (s, 1H), 7.48 (d, J=10.6 Hz, 1H), 6.05 (s, 1H), 5.13 (dd, J=13.2, 5.1 Hz, 1H), 4.55 (d, J=17.4 Hz, 1H), 4.37 (d, J=17.4 Hz, 1H), 3.62-3.32 (m, 8H), 3.04-2.78 (m, 4H), 2.69 (s, 3H), 2.68-2.63 (m, 1H), 2.62-2.53 (m, 4H), 2.47-2.45 (m, 1H), 2.43-2.39 (m, 1H), 2.37-2.27 (m, 2H), 2.08-1.96 (m, 1H).

Prep-HPLC conditions: Column: Welch Utimate AQ-C18, 50*250 mm*10 μm; Mobile Phase A: water (0.05% TFA), Mobile Phase B: ACN; Flow rate: 100 mL/min; Gradient: 20% B to 32% B in 20 min, 32% B; Wave Length: 254 nm.

### Example S102. N-[2-[4-[2-(2,6-dioxo-3-piperidyl)-4,6-difluoro-1-oxo-isoindolin-5-yl]-4-hydroxy-1-piperidyl]ethyl]-N-methyl-benzenesulfonamide (Compound 40)

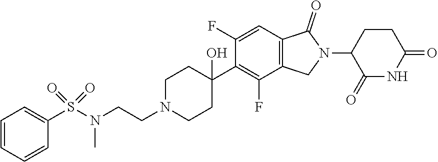 [SMILES: CN(CCN1CCC(O)(c2c(F)cc3c(c2F)CN(C2CCC(=O)NC2=O)C3=O)CC1)S(=O)(=O)c1ccccc1]

The title compound was prepared in 25.7% yield as a brown solid according to the preparation of Example S101 using benzenesulfonyl chloride in step 3.

MS: m/z: Calc'd for C₂₇H₃₀F₂N₄O₆S [M+H]⁺ 577; Found 577. ¹H NMR (300 MHZ, DMSO-d₆) δ 11.03 (s, 1H), 9.55 (s, 1H), 7.99-7.81 (m, 2H), 7.80-7.59 (m, 3H), 7.49 (d, J=10.6 Hz, 1H), 6.05 (s, 1H), 5.13 (dd, J=13.2, 5.0 Hz, 1H), 4.55 (d, J=17.4 Hz, 1H), 4.38 (d, J=17.4 Hz, 1H), 3.62-3.49 (m, 2H), 3.4-3.15 (m, 6H), 3.10-2.84 (m, 1H), 2.74 (s, 3H), 2.67-2.53 (m, 2H), 2.47-2.39 (m, 2H), 2.39-2.26 (m, 2H), 2.15-1.86 (m, 1H).

Prep-HPLC conditions: Column: Welch Utimate AQ-C18, 50*250 mm*10 μm; Mobile Phase A: water (0.05% TFA), Mobile Phase B: ACN; Flow rate: 100 mL/min; Gradient: 20% B to 48% B in 20 min, 48% B; Wave Length: 254 nm.

### Example S105. 2-[2-(2,6-dioxo-3-piperidyl)-4,6-difluoro-1-oxo-isoindoline-5-carbonyl]-3,4-dihydro-1H-isoquinoline-7-carbonitrile (Compound 70)

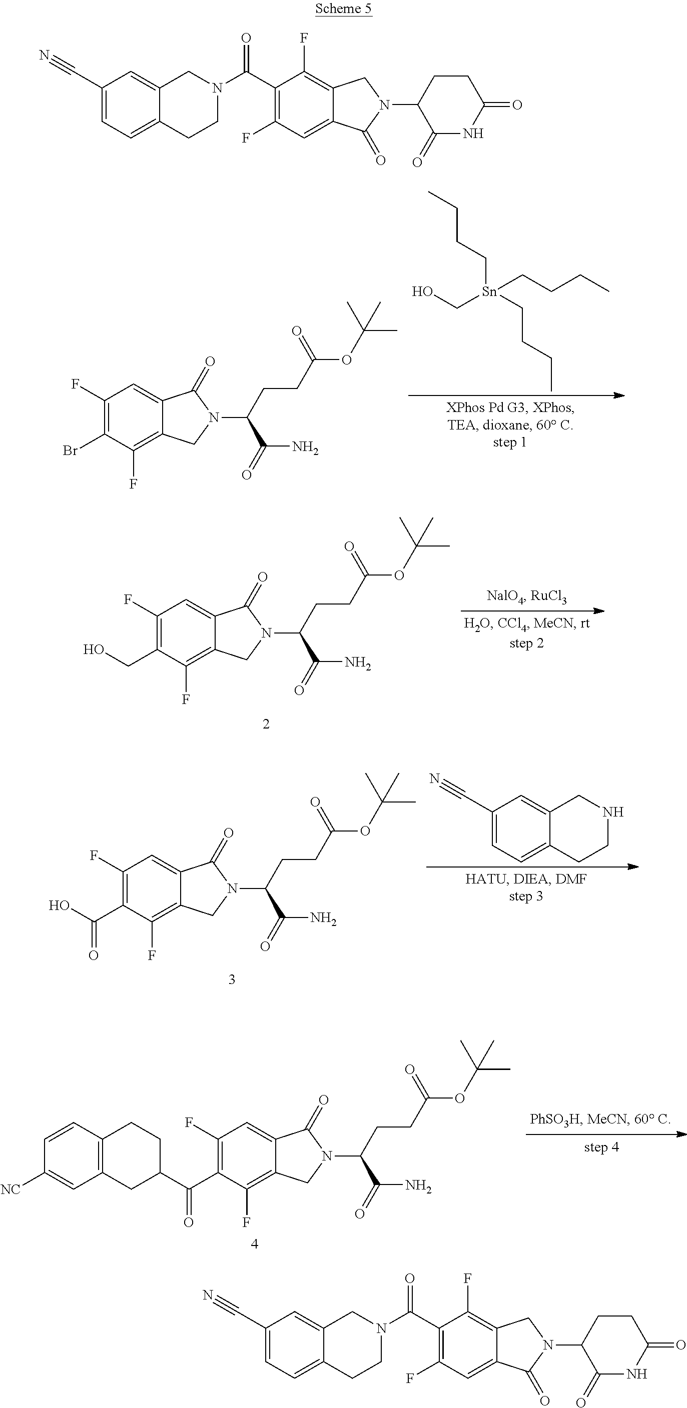

Step 1: To a stirred solution of tert-butyl (4S)-5-amino-4-(5-bromo-4,6-difluoro-1-oxo-isoindolin-2-yl)-5-oxo-pentanoate (500 mg, 1.15 mmol) in 1,4-dioxane (5 mL) was added tributylstannylmethanol (741.1 mg, 2.31 mmol), XPhos Pd G3 (105.68 mg, 0.12 mmol), XPhos (55.1 mg, 0.12 mmol) and TEA (298.3 mg, 2.31 mmol). The resulting solution was degassed three times with nitrogen and stirred at 60° C. for overnight. The reaction was monitored by LCMS. The mixture was concentrated under reduced pressure and applied on a silica gel column with petroleum ether/EtOAc (2/1) to afford tert-butyl (4S)-5-amino-4-[4,6-difluoro-5-(hydroxymethyl)-1-oxo-isoindolin-2-yl]-5-oxo-pentanoate (205 mg, 0.53 mmol, 46.2% yield) as a white solid.

MS: m/z: Calc'd for C₁₈H₂₂F₂N₂O₅ [M+H]⁺ 385; Found 385.

Step 2: To a stirred solution of tert-butyl (4S)-5-amino-4-[4,6-difluoro-5-(hydroxymethyl)-1-oxo-isoindolin-2-yl]-5-oxo-pentanoate (180 mg, 0.47 mmol) in MeCN (2 mL) and CCl₄ (2 mL) was added NaIO₄ (251 mg, 1.4 mmol) and RuCl₃ (23.9 mg, 0.09 mmol) in water (1 mL). The resulting solution was stirred at room temperature for overnight. The reaction was monitored by LCMS. The mixture was concentrated under reduced pressure. The residue was diluted with DCM (50 mL), poured into ice water (50 mL) and extracted with DCM (3×50 mL). The combined organic layers were dried over Na₂SO₄, concentrated under reduced pressure. The crude product was purified reverse flash chromatography with the following conditions (column, C18 silica gel; mobile phase, ACN and water (0.05% TFA), 10% ACN to 30% ACN gradient in 10 min; detector, UV 254 nm) to afford 2-[(1S)-4-tert-butoxy-1-carbamoyl-4-oxo-butyl]-4,6-difluoro-1-oxo-isoindoline-5-carboxylic acid (110 mg, 0.28 mmol, 58.9% yield) as a white solid.

MS: m/z: Calc'd for C₁₈H₂₀F₂N₂O₆ [M+H]⁺ 399; Found 399.

Step 3: To a solution of 2-[(1S)-4-tert-butoxy-1-carbamoyl-4-oxo-butyl]-4,6-difluoro-1-oxo-isoindoline-5-carboxylic acid (50 mg, 0.13 mmol) in DMF (2 mL) was added DIEA (75 mg, 0.63 mmol) and HATU (71.6 mg, 0.19 mmol). To above mixture was added 1,2,3,4-tetrahydroisoquinoline-7-carbonitrile (39.7 mg, 0.25 mmol). The resulting mixture was stirred at 25° C. for overnight. The reaction was monitored by LCMS. After the reaction was complete, the final reaction solution was extracted by Ethyl Acetate (3×50 mL), washed by water (3×50 mL) and dried over anhydrous Na₂SO₄. After filtration, the organic layer was concentrated under reduced pressure. The crude product was applied on a silica gel column with petroleum ether/EtOAc (1/3) to afford tert-butyl (4S)-5-amino-4-[5-(7-cyano-3,4-dihydro-1H-isoquinoline-2-carbonyl)-4,6-difluoro-1-oxo-isoindolin-2-yl]-5-oxo-pentanoate (60 mg, 0.11 mmol, 88.7% yield) as a light yellow solid.

MS: m/z: Calc'd for C₂₈H₂₈F₂N₄O₅ [M+H]⁺ 539; Found 539.

Step 4: To a solution of tert-butyl (4S)-5-amino-4-[5-(7-cyano-3,4-dihydro-1H-isoquinoline-2-carbonyl)-4,6-difluoro-1-oxo-isoindolin-2-yl]-5-oxo-pentanoate (55 mg, 0.09 mmol) in MeCN (3 mL) was added benzenesulfonic acid (44.1 mg, 0.28 mmol). The resulting mixture was degassed three times with nitrogen and then stirred at 60° C. for overnight under nitrogen atmosphere. The reaction was monitored by LCMS. The mixture was concentrated under reduced pressure. The pH value of the solution was adjusted to 9 with saturated sodium bicarbonate solution and extracted with EtOAc (3×50 mL). The combined organic layers were dried over Na₂SO₄ and concentrated under reduced pressure. The crude product was purified by Prep-HPLC to afford 2-[2-(2,6-dioxo-3-piperidyl)-4,6-difluoro-1-oxo-isoindoline-5-carbonyl]-3,4-dihydro-1H-isoquinoline-7-carbonitrile (12.3 mg, 0.026 mmol, 28.4% yield) as a white solid.

MS: m/z: Calc'd for C₂₄H₁₈F₂N₄O₄ [M+H]⁺ 465; Found 465. ¹H NMR (300 MHZ, DMSO-d₆) δ 11.05 (s, 1H), 7.90-7.56 (m, 3H), 7.42 (t, J=8.0 Hz, 1H), 5.28-5.04 (m, 1H), 4.92 (s, 1H), 4.68-4.56 (m, 2H), 4.54-4.36 (m, 1H), 4.06-3.84 (m, 1H), 3.64-3.56 (m, 1H), 3.07-2.95 (m, 1H), 2.94-2.85 (m, 2H), 2.68-2.56 (m, 1H), 2.49-2.33 (m, 1H), 2.10-1.93 (m, 1H).

Prep-HPLC conditions: Column: SunFire Prep C18 OBD Column, 19*150 mm, 5 μm; Mobile Phase A: water (0.05% TFA), Mobile Phase B: ACN; Flow rate: 20 mL/min; Gradient: 30% B to 50% B in 6 min, 50% B; Wave Length: 254 nm.

### Example S106. 3-[5-(5-chloro-3,4-dihydro-1H-isoquinoline-2-carbonyl)-4,6-difluoro-1-oxo-isoindolin-2-yl]piperidine-2,6-dione (Compound 71)

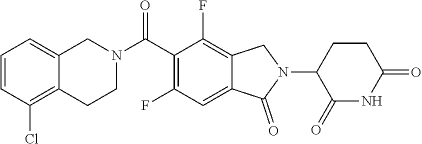 [SMILES: O=C1CCC(N2Cc3c(cc(F)c(C(=O)N4CCc5c(Cl)cccc5C4)c3F)C2=O)C(=O)N1]

The title compound was prepared in 39% overall yield as a white solid according to the preparation of Example S105 using 5-chloro-1,2,3,4-tetrahydroisoquinoline in step 3. MS: m/z: Calc'd for C₂₃H₁₈ClF₂N₃O₄, [M+H]⁺ 474; Found 474. ¹H NMR (400 MHZ, DMSO-d₆) δ 11.06 (d, J=4.8 Hz, 1H), 7.71-7.61 (m, 1H), 7.45-7.04 (m, 3H), 5.23-5.09 (m, 1H), 5.00-4.83 (m, 1H), 4.68-4.53 (m, 3H), 4.09-3.90 (m, 1H), 3.65-3.59 (m, 1H), 2.91-2.85 (m, 2H), 2.78 (d, J=4.9 Hz, 1H), 2.61 (d, J=17.8 Hz, 1H), 2.46-2.43 (m, 1H), 2.06-1.97 (m, 1H).

Prep-HPLC purification conditions: Column: Xselect CSH C18 OBD Column 30*150 mm 5 μm, n; Mobile Phase A: Water (0.05% TFA), Mobile Phase B: ACN; Flow rate: 60 mL/min; Gradient: 27% B to 57% B in 10 min, 57% B; Wave Length: 254 nm.

### Example S107. 3-[5-(6,7-difluoro-3,4-dihydro-1H-isoquinoline-2-carbonyl)-4,6-difluoro-1-oxo-isoindolin-2-yl]piperidine-2,6-dione (Compound 72)

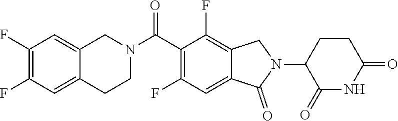 [SMILES: O=C1CCC(N2Cc3c(cc(F)c(C(=O)N4CCc5cc(F)c(F)cc5C4)c3F)C2=O)C(=O)N1]

The title compound was prepared in 22.2% overall yield as a white solid according to the preparation of Example S105 using 6,7-difluoro-1,2,3,4-tetrahydroisoquinoline in step 3.

MS: m/z: Calc'd for C₂₃H₁₇F₄N₃O₄, [M+H]⁺476; Found 476. ¹H NMR (300 MHZ, DMSO-d₆) δ 11.05 (d, J=3.1 Hz, 1H), 7.66-7.63 (m, 1H), 7.52-7.14 (m, 2H), 5.24-5.07 (m, 1H), 4.84 (s, 1H), 4.68-4.55 (m, 2H), 4.49-4.43 (m, 1H), 4.04-3.79 (m, 1H), 3.66-3.49 (m, 1H), 3.05-2.82 (m, 2H), 2.80-2.69 (m, 1H), 2.69-2.50 (m, 1H), 2.50-2.35 (m, 1H), 2.09-1.90 (m, 1H).

Prep-HPLC purification conditions: Column: Xselect CSH C18 OBD Column 30*150 mm 5 μm, n; Mobile Phase A: Water (0.05% TFA), Mobile Phase B: ACN; Flow rate: 60 mL/min; Gradient: 23% B to 53% B in 10 min, 53% B; Wave Length: 254 nm.

### Example S108. 3-[4,6-difluoro-5-[2-(methylamino)-5,7-dihydropyrrolo[3,4-b]pyridine-6-carbonyl]-1-oxo-isoindolin-2-yl]piperidine-2,6-dione (Compound 73)

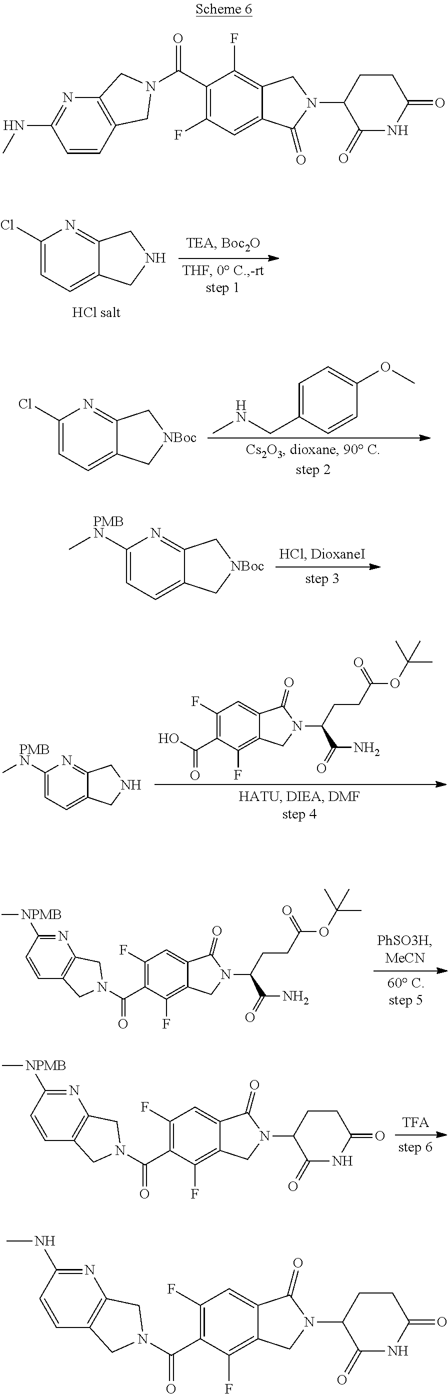

Step 1: To a solution of 2-chloro-6,7-dihydro-5H-pyrrolo[3,4-b]pyridine; hydrochloride (500 mg, 2.62 mmol) and TEA (1.37 mL, 7.85 mmol) in THF (25 mL) was added Boc₂O (628.3 mg, 2.88 mmol) at 0° C. The solution was stirred for 2 hours at room temperature. Desired product could be detected by LCMS. After completion, the mixture was concentrated in vacuum and diluted with water (50 mL) and extracted with DCM (3×80 mL). The combined organic layers were dried over Na₂SO₄ and concentrated under reduced pressure. The residue was purified by silica gel chromatography (petroleum ether:ethyl acetate=8:1) to afford tert-butyl 2-chloro-5,7-dihydropyrrolo[3,4-b]pyridine-6-carboxylate (469 mg, 1.84 mmol, 70.36% yield) as a brown solid.

MS: m/z: Calc'd for C₁₂H₁₅ClN₂O₂ [M+H]⁺ 255; Found 255.

Step 2: To a solution of tert-butyl 2-chloro-5,7-dihydropyrrolo[3,4-b]pyridine-6-carboxylate (300 mg, 1.18 mmol) and 1-(4-methoxyphenyl)-N-methyl-methanamine (178.1 mg, 1.18 mmol) in 1,4-dioxane (20 mL) was added Cs₂CO₃ (1148.4 mg, 3.53 mmol), Ruphos (55 mg, 0.12 mmol) and Ruphos Pd G3 (109.8 mg, 0.12 mmol). The solution was degassed three times with nitrogen and stirred for overnight at 90° C. Desired product could be detected by LCMS. After completion, the mixture was concentrated in vacuum and diluted with water (50 mL) and extracted with DCM (3×50 mL). The combined organic layers were dried over Na₂SO₄ and concentrated under reduced pressure. The residue was purified by silica gel chromatography (petroleum ether:ethyl acetate=10:1) to afford tert-butyl2-[(4-methoxyphenyl)methyl-methyl-amino]-5,7-dihydropyrrolo[3,4-b]pyridine-6-carboxylate (370 mg, 1 mmol, 85% yield) as a yellow solid.

MS: m/z: Calc'd for C₂₁H₂₇N₃O₃ [M+H]⁺ 370; Found 370.

Step 3: To a solution of tert-butyl 2-[(4-methoxyphenyl)methyl-methyl-amino]-5,7-dihydropyrrolo[3,4-b]pyridine-6-carboxylate (370 mg, 1 mmol) in DCM (20 mL) was added 4M HCl in 1,4-dioxane. The solution was stirred for 2 hours under the room temperature. Desired product could be detected by LCMS. After completion, concentrated under reduced pressure get the afford N-[(4-methoxyphenyl)methyl]-N-methyl-6,7-dihydro-5H-pyrrolo[3,4-b]pyridin-2-amine (230 mg crude) as a yellow solid.

MS: m/z: Calc'd for C₁₆H₁₉N₃O [M+H]⁺ 270; Found 270.

Step 4: To a solution of N-[(4-methoxyphenyl)methyl]-N-methyl-6,7-dihydro-5H-pyrrolo[3,4-b]pyridin-2-amine (81.1 mg, 0.3 mmol) and 2-[(1S)-4-tert-butoxy-1-carbamoyl-4-oxo-butyl]-4,6-difluoro-1-oxo-isoindoline-5-carboxylic acid (100 mg, 0.25 mmol) in DMF (5 mL) was added HATU (143.2 mg, 0.38 mmol) and DIEA (0.06 mL, 0.75 mmol). The solution was stirred for 3 hours at 30° C. under nitrogen atmosphere. Desired product could be detected by LCMS. After completion, the mixture was concentrated in vacuum and diluted with water (50 mL) and extracted with DCM (3×80 mL). The combined organic layers were dried over Na₂SO₄ and concentrated under reduced pressure. The residue was purified by silica gel chromatography (petroleum ether:ethyl acetate=8:1) to afford tert-butyl (4S)-5-amino-4-[4,6-difluoro-5-[2-[(4-methoxyphenyl)methyl-methyl-amino]-5,7-dihydropyrrolo[3,4-b]pyridine-6-carbonyl]-1-oxo-isoindolin-2-yl]-5-oxo-pentanoate (106 mg, 0.15 mmol, 61.3% yield) as a white solid.

MS: m/z: Calc'd for C₃₄H₃₇F₂N₅O₆ [M+H]⁺ 650; Found 650.

Step 5: A solution of tert-butyl (4S)-5-amino-4-[4,6-difluoro-5-[2-[(4-methoxyphenyl)methyl-methyl-amino]-5,7-dihydropyrrolo[3,4-b]pyridine-6-carbonyl]-1-oxo-isoindolin-2-yl]-5-oxo-pentanoate (100 mg, 0.15 mmol) and benzenesulfonic acid (72.9 mg, 0.46 mmol) in MeCN (8 mL) was stirred at 60° C. under nitrogen atmosphere for 24 hours. Desired product could be detected by LCMS. The mixture was concentrated in vacuum and diluted with water (50 mL). The resulting mixture was basified to PH 8 with aqueous Na₂CO₃ and extracted with DCM (3×80 mL). The combined organic layers were dried over Na₂SO₄ and concentrated under reduced pressure to afford 3-[4,6-difluoro-5-[2-[(4-methoxyphenyl)methyl-methyl-amino]-5,7-dihydropyrrolo[3,4-b]pyridine-6-carbonyl]-1-oxo-isoindolin-2-yl]piperidine-2,6-dione (80 mg crude) as a white solid.

MS: m/z: Calc'd for C₃₀H₂₇F₂N₅O₅ [M+H]⁺ 576; Found 576.

Step 6: A solution of 3-[4,6-difluoro-5-[2-[(4-methoxyphenyl)methyl-methyl-amino]-5,7-dihydropyrrolo[3,4-b]pyridine-6-carbonyl]-1-oxo-isoindolin-2-yl]piperidine-2,6-dione (75 mg, 0.13 mmol) in TFA (4 mL) was stirred at 60° C. under nitrogen atmosphere for 2 hours. Desired product could be detected by LCMS. The resulting solution was concentrated under reduced pressure and purified by Prep-HPLC to afford 3-[4,6-difluoro-5-[2-(methylamino)-5,7-dihydropyrrolo[3,4-b]pyridine-6-carbonyl]-1-oxo-isoindolin-2-yl]piperidine-2,6-dione (32.4 mg, 54.4% yield) as a white solid.

MS: m/z: Calc'd for C₂₄H₁₈F₂N₄O₄ [M+H]⁺ 456; Found 456. ¹H NMR (400 MHZ, DMSO-d₆) δ 11.05 (s, 1H), 7.73-7.65 (m, 1H), 7.58-7.37 (m, 1H), 6.65-6.50 (m, 1H), 5.24-5.09 (m, 1H), 4.76 (d, J=9.3 Hz, 2H), 4.70-4.41 (m, 4H), 2.99-2.90 (m, 1H), 2.85 (s, 2H), 2.75 (d, J=3.0 Hz, 1H), 2.70-2.57 (m, 1H), 2.50-2.44 (m, 1H), 2.10-1.93 (m, 1H).

Prep-HPLC conditions: Column: SunFire Prep C18 OBD Column, 19*150 mm, 5 μm; Mobile Phase A: water (0.05% TFA), Mobile Phase B: ACN; Flow rate: 20 mL/min; Gradient: 12% B to 15% B in 6 min, 15% B; Wave Length: 254/210 nm.

### Example S116. N-[[2-(2,6-dioxo-3-piperidyl)-4,6-difluoro-1-oxo-isoindolin-5-yl]methyl]indolizine-2-carboxamide (Compound 69)

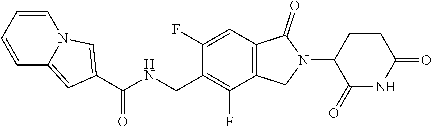 [SMILES: O=C1CCC(N2Cc3c(cc(F)c(CNC(=O)c4cc5ccccn5c4)c3F)C2=O)C(=O)N1]

The title compound was prepared in 21.6% yield as a white solid according to General Scheme 2 using indolizine-2-carboxylic acid, TCFH and NMI in step 4.

MS: m/z: Calc'd for C₂₃H₁₈F₂N₄O₄, [M+H]⁻ 453; Found 453. ¹H NMR (400 MHZ, DMSO-d₆) δ 11.02 (s, 1H), 8.66 (t, J=5.3 Hz, 1H), 8.24 (dd, J=7.0, 1.3 Hz, 1H), 7.95 (d, J=1.6 Hz, 1H), 7.48 (d, J=7.8 Hz, 1H), 7.41 (d, J=9.1 Hz, 1H), 6.78 (s, 1H), 6.75-6.66 (m, 1H), 6.66-6.51 (m, 1H), 5.13 (dd, J=13.3, 5.1 Hz, 1H), 4.75-4.46 (m, 3H), 4.39 (d, J=17.3 Hz, 1H), 3.01-2.82 (m, 1H), 2.67-2.57 (m, 1H), 2.49-2.36 (m, 1H), 2.09-1.95 (m, 1H).

Prep-HPLC purification conditions: Column: SunFire Prep C18 OBD Column, 19*150 mm, 5 μm; Mobile Phase A: water (0.05% TFA), Mobile Phase B: ACN; Flow rate: 20 mL/min; Gradient: 32% B to 32% B in 6 min, 32% B; Wave Length: 254 nm.

### Example S119. 3-(6-fluoro-1-oxo-5-(1-(4-(trifluoromethyl)benzyl) piperidin-4-yl) isoindolin-2-yl) piperidine-2,6-dione (Compound 85)

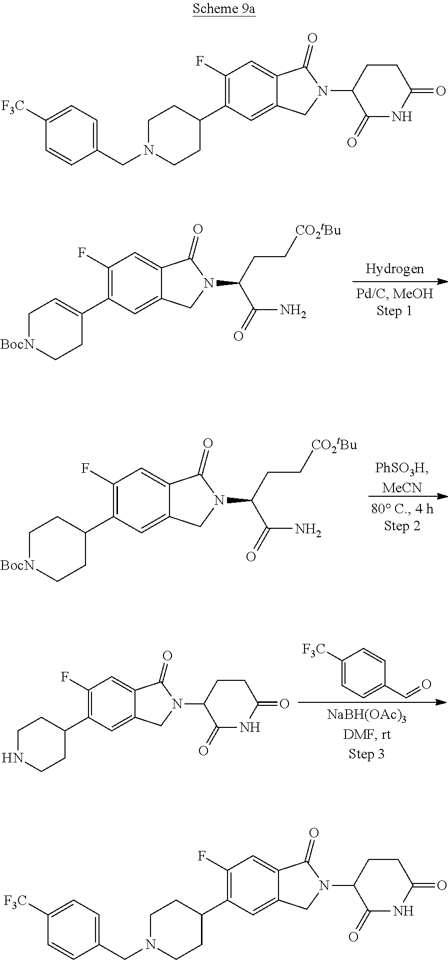

Step 1: A 200 mL round bottom flask was charged with tert-butyl(S)-4-(2-(1-amino-5-(tert-butoxy)-1,5-dioxopentan-2-yl)-6-fluoro-1-oxoisoindolin-5-yl)-3,6-dihydropyridine-1 (2H)-carboxylate (1.000 g, 1.932 mmol), MeOH (50 mL) and Pd/C (0.822 g, 0.386 mmol). The air was replaced with hydrogen and stirred vigorously overnight under an atmosphere of hydrogen. It was filtered through a pad of celite and concentrated to obtain 1.0 g (100% yield) of the desired product.

LC/MS (ESI) m/z 520.5 [(M+H)⁺, calcd for C₂₇H₃₈FN₃O₆ 519.3].

Step 2: A 100 mL round bottom flask was charged with tert-butyl(S)-4-(2-(1-amino-5-(tert-butoxy)-1,5-dioxopentan-2-yl)-6-fluoro-1-oxoisoindolin-5-yl) piperidine-1-carboxylate (1.00 g, 1.925 mmol), benzenesulfonic acid (0.609 g, 3.85 mmol) and MeCN (35 mL). It was heated to 80° C. for 4 h. It was concentrated to dryness, and the residues rinsed with ether to remove the excess PhSO₃H, and dried to obtain the mono-benzenesulfonic acid salt of the desired product (668 mg).

Step 3: To a solution of 4-(trifluoromethyl)benzaldehyde (20.17 mg, 0.116 mmol) and(S)-3-(6-fluoro-1-oxo-5-(piperidin-4-yl) isoindolin-2-yl) piperidine-2,6-dione (20 mg, 0.058 mmol) in DMF (1 mL) was added 2 drops of AcOH, and then stirred at room temperature. After 0.5 h, NaBH(OAc)₃ (36.8 mg, 0.174 mmol) was added and the resulting solution stirred at room temperature for 2 h. It was purified by Preparative HPLC Method 1 to obtain 6.0 mg of the titled compound.

LC/MS (ESI) m/z 504.1 [(M+H)+, calcd for C₂₆H₂₅F₄N₃O₃ 503.2]; HPLC^{a} T_{Ret}=1.29 min; ¹H NMR (500 MHz, DMSO-d₆) δ 10.99 (s, 1H), 7.74-7.67 (m, J=8.2 Hz, 2H), 7.63 (d, J=6.0 Hz, 1H), 7.61-7.55 (m, J=8.2 Hz, 2H), 7.47 (d, J=9.2 Hz, 1H), 5.10 (dd, J=13.3, 5.0 Hz, 1H), 4.46-4.38 (m, 1H), 4.34-4.26 (m, 1H), 3.66-3.58 (m, 1H), 2.97-2.87 (m, 3H), 2.64-2.57 (m, 1H), 2.57-2.56 (m, 1H), 2.52-2.50 (m, 7H), 2.46-2.33 (m, 1H), 2.22-2.09 (m, 2H), 2.04-1.96 (m, 1H)

Examples of compounds prepared by following the procedures outlined for Scheme 9 and Scheme 9a using the appropriate aldehydes are listed in Table 4.

TABLE 4

| | Example | | |
|---|---|---|---|
| Cmpd No | SNo. | Structure | Chemical name |
| 86 | 125 | 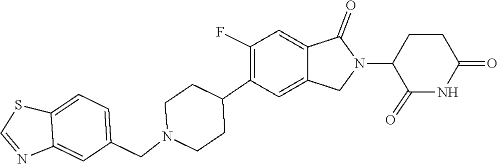 [SMILES: O=C1CCC(N2Cc3cc(C4CCN(Cc5ccc6scnc6c5)CC4)c(F)cc3C2=O)C(=O)N1] | 3-(5-(1-(benzo[d]thiazol-5- ylmethyl)piperidin-4-yl)-6- fluoro-1-oxoisoindolin-2- yl)piperidine-2,6-dione |
| 87 | 126 | 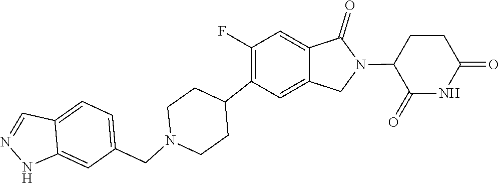 [SMILES: O=C1CCC(N2Cc3cc(C4CCN(Cc5ccc6cn[nH]c6c5)CC4)c(F)cc3C2=O)C(=O)N1] | 3-(5-(1-((1H-indazol-6- yl)methyl)piperidin-4-yl)-6- fluoro-1-oxoisoindolin-2- yl)piperidine-2,6-dione |
| 88 | 127 | 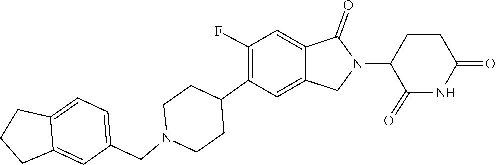 [SMILES: O=C1CCC(N2Cc3cc(C4CCN(Cc5ccc6c(c5)CCC6)CC4)c(F)cc3C2=O)C(=O)N1] | 3-(5-(1-((2,3-dihydro-1H-inden- 5-yl)methyl)piperidin-4-yl)-6- fluoro-1-oxoisoindolin-2- yl)piperidine-2,6-dione |
| 89 | 128 | 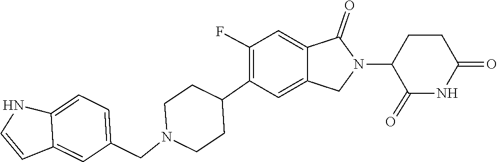 [SMILES: O=C1CCC(N2Cc3cc(C4CCN(Cc5ccc6[nH]ccc6c5)CC4)c(F)cc3C2=O)C(=O)N1] | 3-(5-(1-((1H-indol-5- yl)methyl)piperidin-4-yl)-6- fluoro-1-oxoisoindolin-2- yl)piperidine-2,6-dione |
| 90 | 129 | 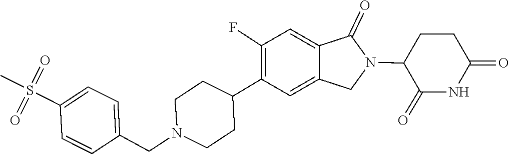 [SMILES: CS(=O)(=O)c1ccc(CN2CCC(c3cc4c(cc3F)C(=O)N(C3CCC(=O)NC3=O)C4)CC2)cc1] | 3-(6-fluoro-5-(1-(4- (methylsulfonyl)benzyl)piperidin- 4-yl)-1-oxoisoindolin-2- yl)piperidine-2,6-dione |
| 91 | 130 | 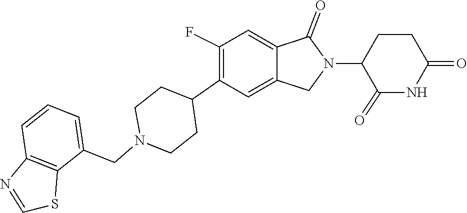 [SMILES: O=C1CCC(N2Cc3cc(C4CCN(Cc5cccc6ncsc56)CC4)c(F)cc3C2=O)C(=O)N1] | 3-(5-(1-(benzo[d]thiazol-7- ylmethyl)piperidin-4-yl)-6- fluoro-1-oxoisoindolin-2- yl)piperidine-2,6-dione |

### Example S131. 3-(5-(1-(benzo[d]thiazol-6-ylmethyl)-4-hydroxypiperidin-4-yl)-6-fluoro-1-oxoisoindolin-2-yl) piperidine-2,6-dione (Compound 35)

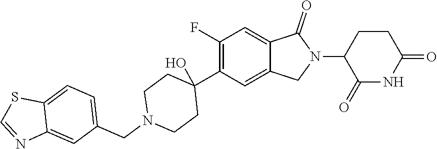 [SMILES: O=C1CCC(N2Cc3cc(C4(O)CCN(Cc5ccc6scnc6c5)CC4)c(F)cc3C2=O)C(=O)N1]

A 1 dram vial was charged with 3-(6-fluoro-5-(4-hydroxypiperidin-4-yl)-1-oxoisoindolin-2-yl) piperidine-2,6-dione, benzenesulfonic acid salt (20 mg, 0.038 mmol) dissolved in 1 mL DMF. To this was added 6-(chloromethyl)benzo[d]thiazole (10.60 mg, 0.058 mmol) followed by the addition of Hunig's base (0.034 mL, 0.192 mmol). The resulting mixture was heated at 80° C. for 1 h. It was cooled to room temperature, and purified by Preparative HPLC Method 1 to obtain 6.0 mg of the titled compound.

LC/MS (ESI) m/z 509.2 [(M+H)⁺, calcd for C₂₆H₂₅FN₄O₄S 508.2]; HPLC^{a} T_{Ret}=0.97 min; ¹H NMR (500 MHz, DMSO-d₆) δ 11.00 (br s, 1H), 9.49 (s, 1H), 8.36 (br s, 1H), 8.20 (br d, J=8.2 Hz, 1H), 7.88 (br d, J=6.1 Hz, 1H), 7.74 (br d, J=8.5 Hz, 1H), 7.54-7.45 (m, 1H), 5.08 (br dd, J=12.4, 4.4 Hz, 1H), 4.63-4.39 (m, 3H), 4.37-4.27 (m, 1H), 3.66-3.49 (m, 2H), 2.95-2.83 (m, 1H), 2.62 (br d, J=16.5 Hz, 1H), 2.51 (br s, 6H), 2.47-2.32 (m, 2H), 2.06-1.99 (m, 1H), 1.90-1.77 (m, 2H).

### Example S132. 3-(5-(1-(benzo[d]thiazol-6-ylmethyl) piperidin-4-yl)-6-fluoro-1-oxoisoindolin-2-yl) piperidine-2,6-dione (Compound 81)

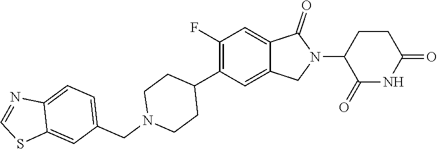 [SMILES: O=C1CCC(N2Cc3cc(C4CCN(Cc5ccc6ncsc6c5)CC4)c(F)cc3C2=O)C(=O)N1]

The titled compound was obtained in 51% yield as an off-white solid by following the alkylation procedure outlined for the synthesis of Example S131 using 3-(6-fluoro-1-oxo-5-(piperidin-4-yl) isoindolin-2-yl) piperidine-2,6-dione as the coupling partner.

LC/MS (ESI) m/z 493.1 [(M+H)⁺, calcd for C₂₆H₂₅FN₄O₃S 492.2]; HPLC^{a} T_{Ret}=1.07 min; ¹H NMR (500 MHz, DMSO-d₆) δ 10.95 (s, 1H), 9.46 (s, 1H), 8.30 (br s, 1H), 8.16 (d, J=8.4 Hz, 1H), 7.67 (br d, J=8.4 Hz, 1H), 7.47 (br d, J=9.3 Hz, 2H), 5.06 (br dd, J=13.0, 4.8 Hz, 1H), 4.51-4.42 (m, 2H), 4.39 (br d, J=17.2 Hz, 1H), 4.31-4.23 (m, 1H), 3.69-3.45 (m, 2H), 3.21-3.04 (m, 2H), 2.91-2.81 (m, 1H), 2.59-2.52 (m, 1H), 2.46 (br s, 4H), 2.39-2.28 (m, 2H), 2.09-1.99 (m, 1H), 1.90-1.77 (m, 2H).

Examples of compounds prepared by following the alkylation procedure outlined for the syntheses of Example S131 and Example S132 using the appropriate alkyl halides are listed in Table 5.

TABLE 5

| Compound | Example | | |
|---|---|---|---|
| No | SNo. | Structure | Chemical name |
| 80 | 133 | 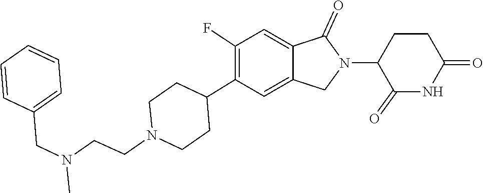 [SMILES: CN(CCN1CCC(c2cc3c(cc2F)C(=O)N(C2CCC(=O)NC2=O)C3)CC1)Cc1ccccc1] | 3-(5-(1-(2- (benzyl(methyl)amino)ethyl) piperidin-4-yl)-6-fluoro-1- oxoisoindolin-2- yl)piperidine-2,6-dione |
| 82 | 134 | 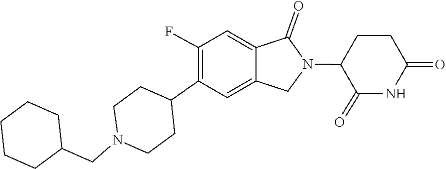 [SMILES: O=C1CCC(N2Cc3cc(C4CCN(CC5CCCCC5)CC4)c(F)cc3C2=O)C(=O)N1] | 3-(5-(1- (cyclohexylmethyl)piperidin- 4-yl)-6-fluoro-1- oxoisoindolin-2- yl)piperidine-2,6-dione |
| 83 | 135 | 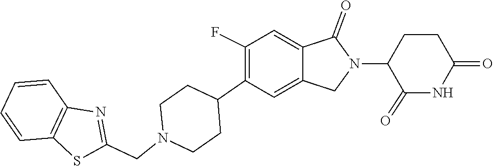 [SMILES: O=C1CCC(N2Cc3cc(C4CCN(Cc5nc6ccccc6s5)CC4)c(F)cc3C2=O)C(=O)N1] | 3-(5-(1-(benzo[d]thiazol-2- ylmethyl)piperidin-4-yl)-6- fluoro-1-oxoisoindolin-2- yl)piperidine-2,6-dione |
| 84 | 136 | 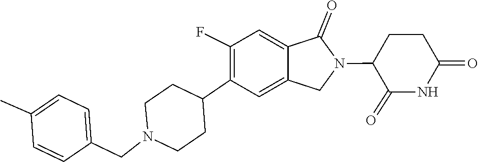 [SMILES: Cc1ccc(CN2CCC(c3cc4c(cc3F)C(=O)N(C3CCC(=O)NC3=O)C4)CC2)cc1] | 3-(6-fluoro-5-(1-(4- methylbenzyl)piperidin-4- yl)-1-oxoisoindolin-2- yl)piperidine-2,6-dione |

### Example S140. 3-(5-(6-amino-4,5-dimethylpyridin-2-yl)-6-fluoro-1-oxoisoindolin-2-yl) piperidine-2,6-dione (Compound 34)

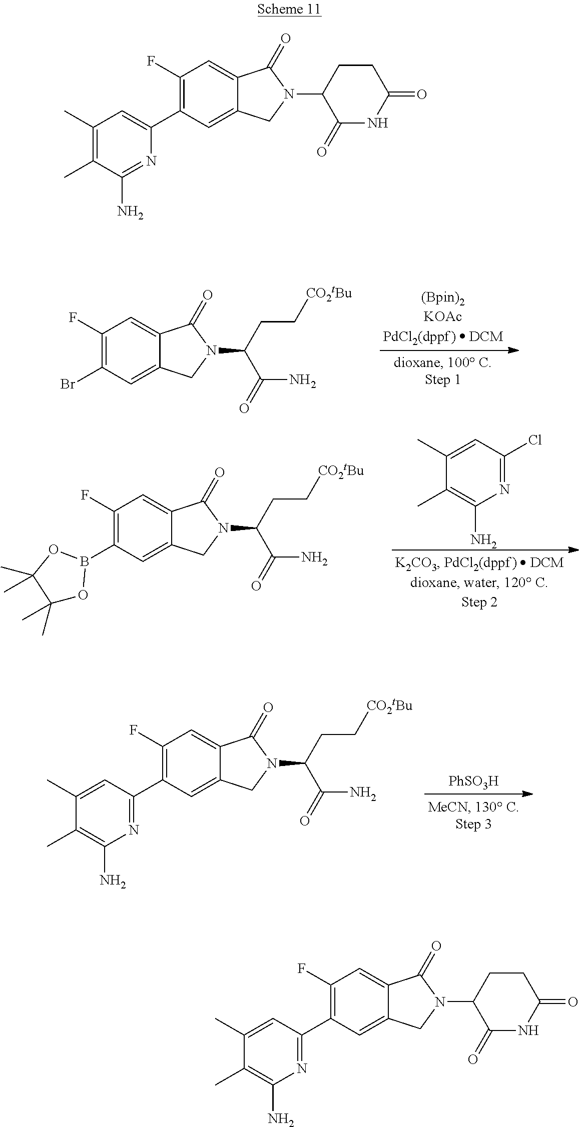

Step 1: To a solution of 4,4,4′,4′,5,5,5′,5′-octamethyl-2,2′-bi (1,3,2-dioxaborolane) (275 mg, 1.084 mmol), potassium acetate (213 mg, 2.167 mmol) and tert-butyl(S)-5-amino-4-(5-bromo-6-fluoro-1-oxoisoindolin-2-yl)-5-oxopentanoate (300 mg, 0.722 mmol) in Dioxane (10 mL) was added PdCl₂ (dppf)·DCM (59.0 mg, 0.072 mmol) and the air replaced with N₂. It was heated to 100° C. for 16 h. It was cooled to room temperature, diluted with EtOAc and quenched with brine and the organic layer separated, dried over Na₂SO₄ and concentrated. The crude was purified by flash column chromatography eluting with 0-6% MeOH/DCM to obtain 302 mg (90% yield) of the desired product.

LC/MS (ESI) m/z 325.1 [(M-137)+, calcd for C₂₃H₃₂BFN₂O₆ 462.2].

Step 2: A 5 mL microwave vial was charged with 6-chloro-3,4-dimethylpyridin-2-amine (16.5 mg, 0.105 mmol) dissolved in Dioxane (3 mL), tert-butyl(S)-5-amino-4-(6-fluoro-1-oxo-5-(4,4,5,5-tetramethyl-1,3,2-dioxaborolan-2-yl) isoindolin-2-yl)-5-oxopentanoate (60.9 mg, 0.132 mmol), PdCl₂ (dtbpf) (3.43 mg, 5.27 μmol), and an aq. solution of K₃PO₄ (0.176 mL, 0.527 mmol). It was sealed and the air was replaced with nitrogen, and then heated for 0.25 h in the microwave at 120° C. It was diluted with EtOAc, washed with brine, and the organic layer separated, dried over MgSO₄ and concentrated to obtain 32 mg of the desired product which was used for the next step without further purification.

LC/MS (ESI) m/z 457.3 [(M+H)⁺, calcd for C₂₄H₂₉FN₄O₄ 456.2].

Step 3: To tert-butyl(S)-5-amino-4-(5-(6-amino-4,5-dimethylpyridin-2-yl)-6-fluoro-1-oxoisoindolin-2-yl)-5-oxopentanoate (27 mg, 0.059 mmol) was added 1 mL solution of PhSO₃H in MeCN (0.25M), and microwaved for 15 min at 120° C. It was purified by Preparative HPLC Method 1 to obtain 11.9 mg (28% yield) of the desired product.

LC/MS (ESI) m/z 383.1 [(M+H)+, calcd for C₂₀H₁₉FN₄O₃ 382.1]; HPLC^{a} T_{Ret}=1.00 min; ¹H NMR (500 MHZ, DMSO-d₆) δ 11.00 (s, 1H), 8.03 (d, J=6.6 Hz, 1H), 7.57 (d, J=9.9 Hz, 1H), 6.93-6.88 (m, 1H), 5.70 (s, 1H), 5.10 (dd, J=13.4, 5.1 Hz, 1H), 4.50 (d, J=17.2 Hz, 1H), 4.37 (d, J=17.0 Hz, 1H), 3.62-3.53 (m, 1H), 2.95-2.84 (m, 1H), 2.68-2.58 (m, 1H), 2.47-2.32 (m, 1H), 2.22 (s, 3H), 2.08-2.04 (m, 1H), 2.03 (s, 3H), 1.22 (br s, 1H)

Additional compounds may be prepared by following the procedures outlined for Scheme 3 and Scheme 4, using the appropriate boronic acids/esters.

### Example S146. 3-(5-(6-amino-4,5-dimethylpyridin-2-yl)-4,6-difluoro-1-oxoisoindolin-2-yl) piperidine-2,6-dione (Compound 93)

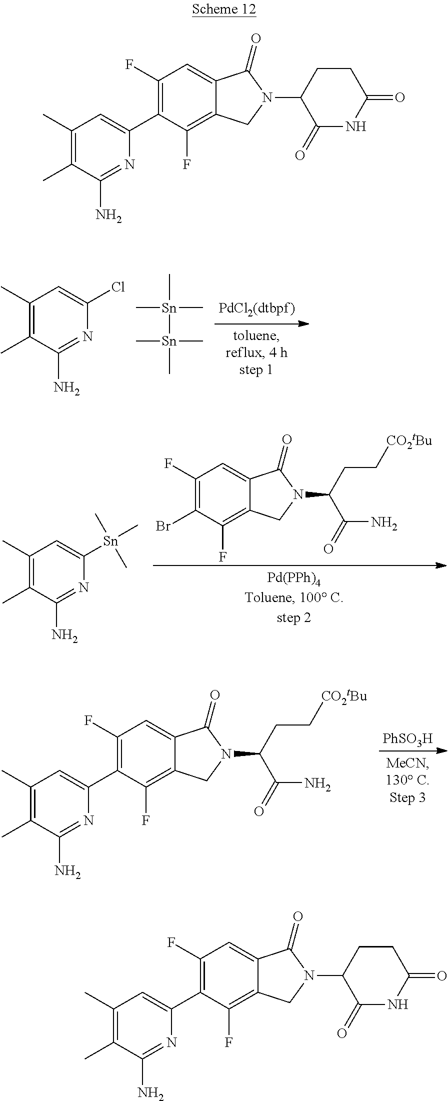

Step 1: A solution of 6-chloro-3,4-dimethylpyridin-2-amine (30.0 mg, 0.192 mmol), 1,1,1,2,2,2-hexamethyldistannane (69.0 mg, 0.211 mmol) and PdCl₂ (dtbpf) (12.48 mg, 0.019 mmol) in toluene (5 mL) and the air was replaced with N₂. It was heated to 110° C. for 4 h. After cooling to room temperature, it was diluted with EtOAc and brine, and the organic layer separated, dried over Na₂SO₄, filtered and concentrated. The crude material (38 mg) was used for the next step without further purification.

LC/MS (ESI) m/z 287.1 [(M+H)+, calcd for C₁₀H₁₈N₂Sn 286.0].

Step 2: To a 2 dram pressure vial was charged with tert-butyl(S)-5-amino-4-(5-bromo-4,6-difluoro-1-oxoisoindolin-2-yl)-5-oxopentanoate (38.0 mg, 0.088 mmol), 3,4-dimethyl-6-(trimethylstannyl)pyridin-2-amine (25 mg, 0.088 mmol), Pd (PPh₃) 4 (10.14 mg, 8.77 μmol) and Toluene (6 mL). The mixture was heated at 100° C. for 16 h under a nitrogen atmosphere. After cooling to room temperature, it was diluted with EtOAc and brine, and the organic layer separated, dried over MgSO₄, filtered and concentrated. The crude material (28 mg) was used for the next step without further purification.

LC/MS (ESI) m/z 475.1 [(M+H)+, calcd for C₂₄H₂₈F₂N₄O₄ 474.2].

Step 3: To tert-butyl(S)-5-amino-4-(5-(6-amino-4,5-dimethylpyridin-2-yl)-4,6-difluoro-1-oxoisoindolin-2-yl)-5-oxopentanoate (28 mg, 0.059 mmol) was added 1 mL solution of PhSO₃H in MeCN (0.25M), and microwaved for 15 min at 120° C. It was purified by Preparative HPLC Method 1 to obtain 6.8 mg (28% yield) of the desired product.

LC/MS (ESI) m/z 401.0 [(M+H)+, calcd for C₂₀H₁₈F₂N₄O₃ 400.1]; HPLC^{a} T_{Ret}=1.04 min; ¹H NMR (500 MHz, DMSO-d₆) δ 11.03 (s, 1H), 7.53 (d, J=7.5 Hz, 1H), 6.58 (s, 1H), 5.80 (s, 2H), 5.15 (dd, J=13.4, 5.0 Hz, 1H), 4.60 (d, J=17.2 Hz, 1H), 4.43 (d, J=17.3 Hz, 1H), 2.97-2.89 (m, 1H), 2.62 (br d, J=18.1 Hz, 1H), 2.49-2.41 (m, 1H), 2.22 (s, 3H), 2.06 (br s, 1H), 2.04 (s, 3H)

### Example S147. 3-(4,6-difluoro-1-oxo-5-(5,6,7,8-tetrahydro-1,8-naphthyridin-2-yl) isoindolin-2-yl) piperidine-2,6-dione (Compound 94)

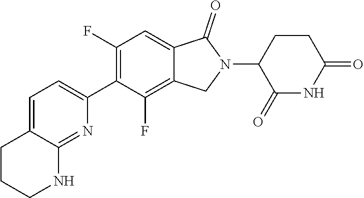 [SMILES: O=C1CCC(N2Cc3c(cc(F)c(-c4ccc5c(n4)NCCC5)c3F)C2=O)C(=O)N1]

The titled compound was synthesized from 7-chloro-1,2,3,4-tetrahydro-1,8-naphthyridine by following the route outlined for the synthesis of Example S146.

LC/MS (ESI) m/z 413.1 [(M+H)+, calcd for C₂₁H₁₈F₂N₄O₃ 412.1]; HPLC^{a} T_{Ret}=1.05 min; ¹H NMR (500 MHz, DMSO-d₆) δ 11.02 (s, 1H), 7.53 (d, J=7.6 Hz, 1H), 7.29 (d, J=7.2 Hz, 1H), 6.65 (br s, 1H), 6.58 (d, J=7.2 Hz, 1H), 5.14 (dd, J=13.2, 4.9 Hz, 1H), 4.60 (d, J=17.2 Hz, 1H), 4.43 (d, J=17.2 Hz, 1H), 2.97-2.89 (m, 1H), 2.73 (br t, J=6.1 Hz, 2H), 2.62 (br dd, J=15.6, 2.2 Hz, 1H), 2.56-2.53 (m, 2H), 2.49-2.41 (m, 1H), 2.08-2.01 (m, 1H), 1.86-1.79 (m, 2H)

### Example S148 Includes Compounds Made According to General Scheme 1

TABLE 6

| Compound | | |
|---|---|---|
| No | Structure | Chemical name |
| 1 | 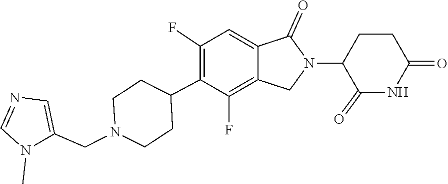 [SMILES: Cn1cncc1CN1CCC(c2c(F)cc3c(c2F)CN(C2CCC(=O)NC2=O)C3=O)CC1] | 3-(4,6-difluoro-5-(1-((1- methyl-1H-imidazol-5- yl)methyl)piperidin-4-yl)-1- oxoisoindolin-2-yl)piperidine- 2,6-dione |
| 2 | 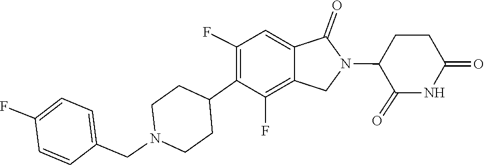 [SMILES: O=C1CCC(N2Cc3c(cc(F)c(C4CCN(Cc5ccc(F)cc5)CC4)c3F)C2=O)C(=O)N1] | 3-(4,6-difluoro-5-(1-(4- fluorobenzyl)piperidin-4-yl)- 1-oxoisoindolin-2- yl)piperidine-2,6-dione |
| 3 | 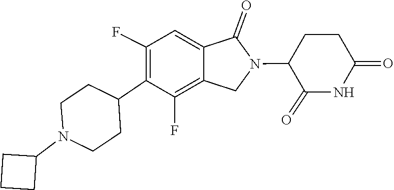 [SMILES: O=C1CCC(N2Cc3c(cc(F)c(C4CCN(C5CCC5)CC4)c3F)C2=O)C(=O)N1] | 3-(5-(1-cyclobutylpiperidin-4- yl)-4,6-difluoro-1- oxoisoindolin-2-yl)piperidine- 2,6-dione |
| 4 | 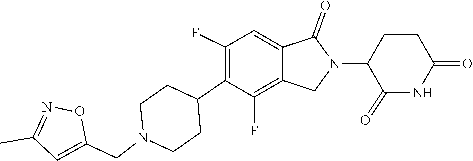 [SMILES: Cc1cc(CN2CCC(c3c(F)cc4c(c3F)CN(C3CCC(=O)NC3=O)C4=O)CC2)on1] | 3-(4,6-difluoro-5-(1-((3- methylisoxazol-5- yl)methyl)piperidin-4-yl)-1- oxoisoindolin-2-yl)piperidine- 2,6-dione |
| 5 | 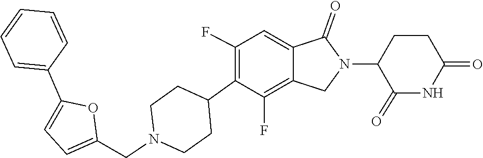 [SMILES: O=C1CCC(N2Cc3c(cc(F)c(C4CCN(Cc5ccc(-c6ccccc6)o5)CC4)c3F)C2=O)C(=O)N1] | 3-(4,6-difluoro-1-oxo-5-(1- ((5-phenylfuran-2- yl)methyl)piperidin-4- yl)isoindolin-2-yl)piperidine- 2,6-dione |
| 6 | 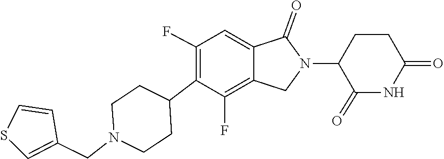 [SMILES: O=C1CCC(N2Cc3c(cc(F)c(C4CCN(Cc5ccsc5)CC4)c3F)C2=O)C(=O)N1] | 3-(4,6-difluoro-1-oxo-5-(1- (thiophen-3- ylmethyl)piperidin-4- yl)isoindolin-2-yl)piperidine- 2,6-dione |
| 7 | 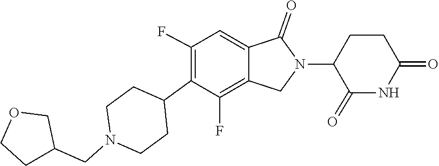 [SMILES: O=C1CCC(N2Cc3c(cc(F)c(C4CCN(CC5CCOC5)CC4)c3F)C2=O)C(=O)N1] | 3-(4,6-difluoro-1-oxo-5-(1- ((tetrahydrofuran-3- yl)methyl)piperidin-4- yl)isoindolin-2-yl)piperidine- 2,6-dione |
| 8 | 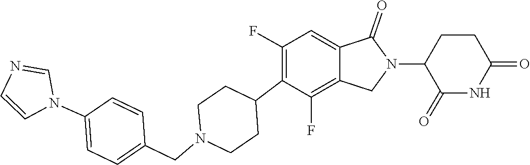 [SMILES: O=C1CCC(N2Cc3c(cc(F)c(C4CCN(Cc5ccc(-n6ccnc6)cc5)CC4)c3F)C2=O)C(=O)N1] | 3-(5-(1-(4-(1H-imidazol-1- yl)benzyl)piperidin-4-yl)-4,6- difluoro-1-oxoisoindolin-2- yl)piperidine-2,6-dione |
| 9 | 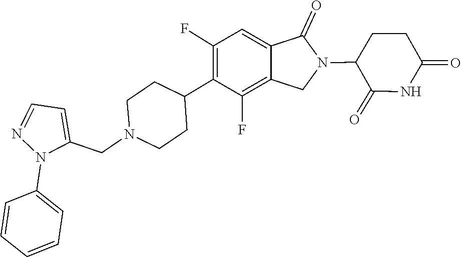 [SMILES: O=C1CCC(N2Cc3c(cc(F)c(C4CCN(Cc5ccnn5-c5ccccc5)CC4)c3F)C2=O)C(=O)N1] | 3-(4,6-difluoro-1-oxo-5-(1- ((1-phenyl-1H-pyrazol-5- yl)methyl)piperidin-4- yl)isoindolin-2-yl)piperidine- 2,6-dione |
| 10 | 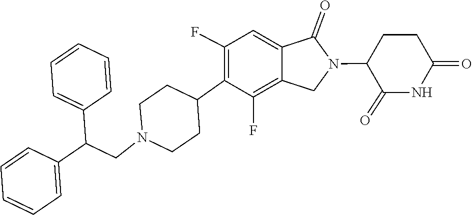 [SMILES: O=C1CCC(N2Cc3c(cc(F)c(C4CCN(CC(c5ccccc5)c5ccccc5)CC4)c3F)C2=O)C(=O)N1] | 3-(5-(1-(2,2- diphenylethyl)piperidin-4-yl)- 4,6-difluoro-1-oxoisoindolin- 2-yl)piperidine-2,6-dione |

### Example S149 Includes the Following Compounds, which were Made According to the Specified General Procedure

TABLE 7

| | | Procedure |
|---|---|---|
| Compound | | General |
| number | Structure | Scheme |
| 16 | 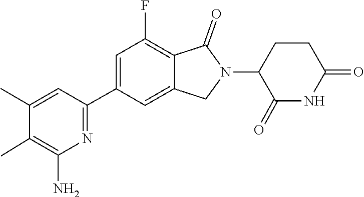 [SMILES: Cc1cc(-c2cc(F)c3c(c2)CN(C2CCC(=O)NC2=O)C3=O)nc(N)c1C] | 4 |
| 20 | 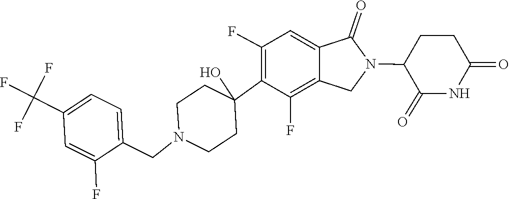 [SMILES: O=C1CCC(N2Cc3c(cc(F)c(C4(O)CCN(Cc5ccc(C(F)(F)F)cc5F)CC4)c3F)C2=O)C(=O)N1] | 1 |
| 36 | 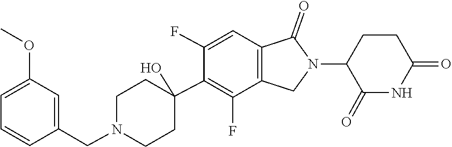 [SMILES: COc1cccc(CN2CCC(O)(c3c(F)cc4c(c3F)CN(C3CCC(=O)NC3=O)C4=O)CC2)c1] | 1 |
| 46 | 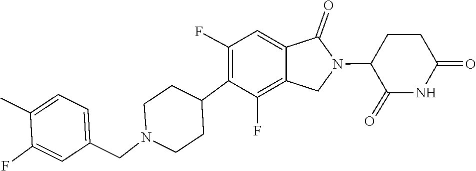 [SMILES: Cc1ccc(CN2CCC(c3c(F)cc4c(c3F)CN(C3CCC(=O)NC3=O)C4=O)CC2)cc1F] | 1 |
| 74 | 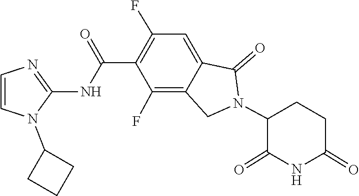 [SMILES: O=C1CCC(N2Cc3c(cc(F)c(C(=O)Nc4nccn4C4CCC4)c3F)C2=O)C(=O)N1] | 3 |
| 75 | 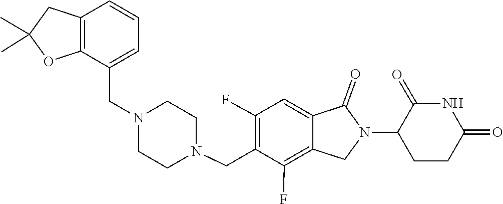 [SMILES: CC1(C)Cc2cccc(CN3CCN(Cc4c(F)cc5c(c4F)CN(C4CCC(=O)NC4=O)C5=O)CC3)c2O1] | 2 |
| 76 | 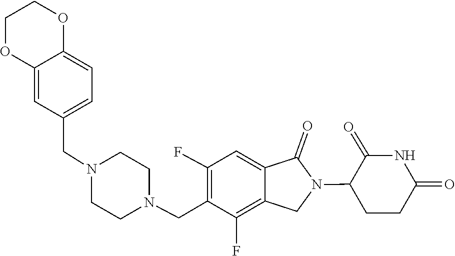 [SMILES: O=C1CCC(N2Cc3c(cc(F)c(CN4CCN(Cc5ccc6c(c5)OCCO6)CC4)c3F)C2=O)C(=O)N1] | 2 |
| 77 | 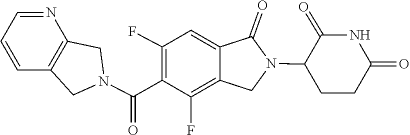 [SMILES: O=C1CCC(N2Cc3c(cc(F)c(C(=O)N4Cc5cccnc5C4)c3F)C2=O)C(=O)N1] | 3 |
| 95 | 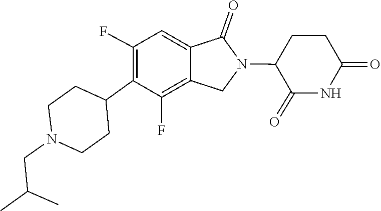 [SMILES: CC(C)CN1CCC(c2c(F)cc3c(c2F)CN(C2CCC(=O)NC2=O)C3=O)CC1] | 1 |
| 96 | 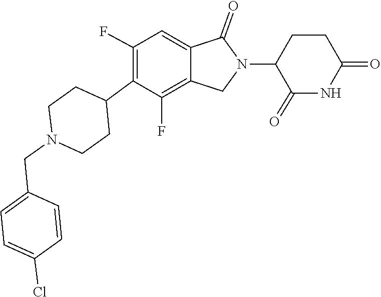 [SMILES: O=C1CCC(N2Cc3c(cc(F)c(C4CCN(Cc5ccc(Cl)cc5)CC4)c3F)C2=O)C(=O)N1] | 1 |
| 97 | 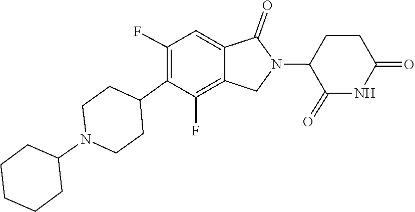 [SMILES: O=C1CCC(N2Cc3c(cc(F)c(C4CCN(C5CCCCC5)CC4)c3F)C2=O)C(=O)N1] | 1 |
| 98 | 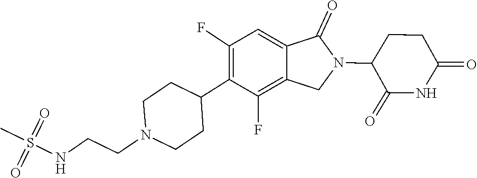 [SMILES: CS(=O)(=O)NCCN1CCC(c2c(F)cc3c(c2F)CN(C2CCC(=O)NC2=O)C3=O)CC1] | 1 |
| 99 | 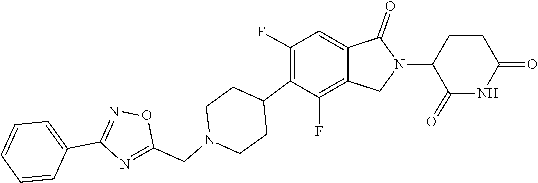 [SMILES: O=C1CCC(N2Cc3c(cc(F)c(C4CCN(Cc5nc(-c6ccccc6)no5)CC4)c3F)C2=O)C(=O)N1] | 1 |
| 100 | 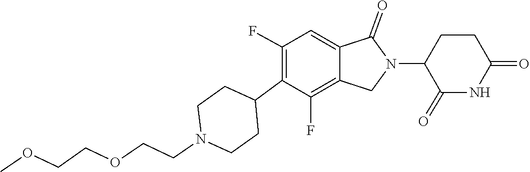 [SMILES: COCCOCCN1CCC(c2c(F)cc3c(c2F)CN(C2CCC(=O)NC2=O)C3=O)CC1] | 1 |
| 101 | 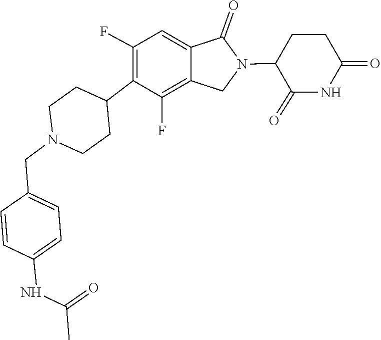 [SMILES: CC(=O)Nc1ccc(CN2CCC(c3c(F)cc4c(c3F)CN(C3CCC(=O)NC3=O)C4=O)CC2)cc1] | 1 |
| 102 | 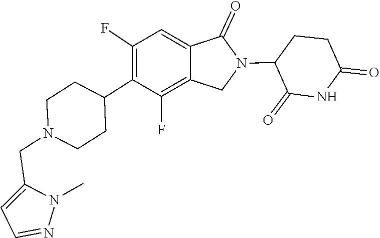 [SMILES: Cn1nccc1CN1CCC(c2c(F)cc3c(c2F)CN(C2CCC(=O)NC2=O)C3=O)CC1] | 1 |
| 103 | 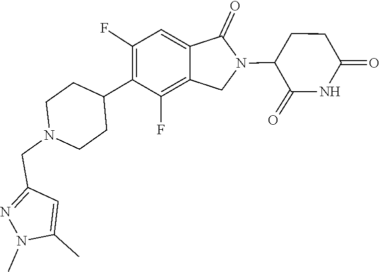 [SMILES: Cc1cc(CN2CCC(c3c(F)cc4c(c3F)CN(C3CCC(=O)NC3=O)C4=O)CC2)nn1C] | 1 |
| 104 | 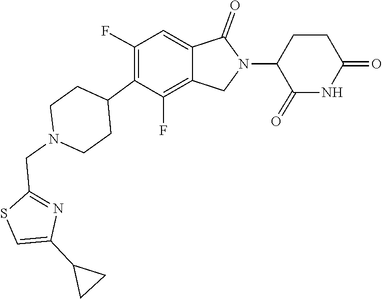 [SMILES: O=C1CCC(N2Cc3c(cc(F)c(C4CCN(Cc5nc(C6CC6)cs5)CC4)c3F)C2=O)C(=O)N1] | 1 |
| 105 | 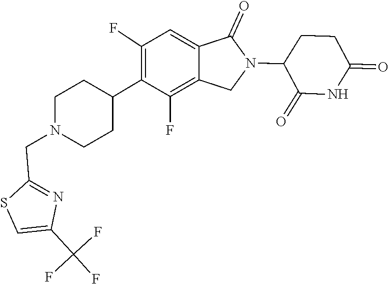 [SMILES: O=C1CCC(N2Cc3c(cc(F)c(C4CCN(Cc5nc(C(F)(F)F)cs5)CC4)c3F)C2=O)C(=O)N1] | 1 |
| 106 | 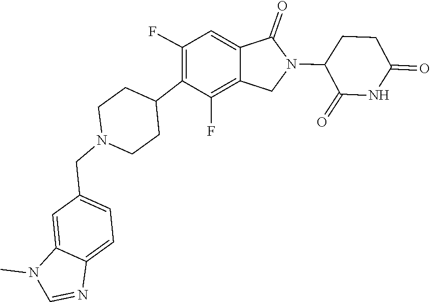 [SMILES: Cn1cnc2ccc(CN3CCC(c4c(F)cc5c(c4F)CN(C4CCC(=O)NC4=O)C5=O)CC3)cc21] | 1 |
| 107 | 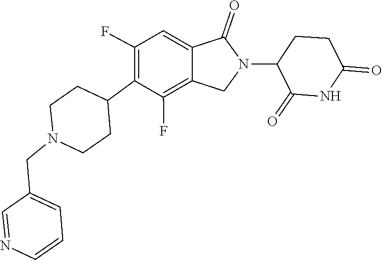 [SMILES: O=C1CCC(N2Cc3c(cc(F)c(C4CCN(Cc5cccnc5)CC4)c3F)C2=O)C(=O)N1] | 1 |
| 108 | 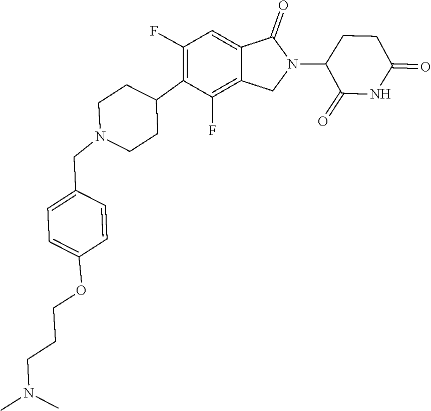 [SMILES: CN(C)CCCOc1ccc(CN2CCC(c3c(F)cc4c(c3F)CN(C3CCC(=O)NC3=O)C4=O)CC2)cc1] | 1 |
| 109 | 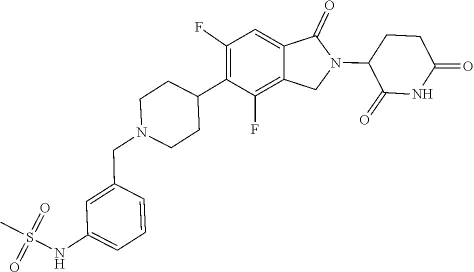 [SMILES: CS(=O)(=O)Nc1cccc(CN2CCC(c3c(F)cc4c(c3F)CN(C3CCC(=O)NC3=O)C4=O)CC2)c1] | 1 |
| 110 | 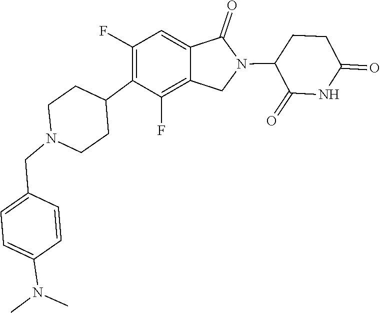 [SMILES: CN(C)c1ccc(CN2CCC(c3c(F)cc4c(c3F)CN(C3CCC(=O)NC3=O)C4=O)CC2)cc1] | 1 |
| 111 | 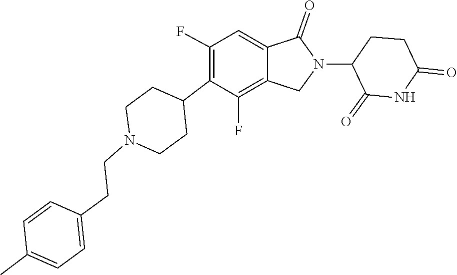 [SMILES: Cc1ccc(CCN2CCC(c3c(F)cc4c(c3F)CN(C3CCC(=O)NC3=O)C4=O)CC2)cc1] | 1 |
| 112 | 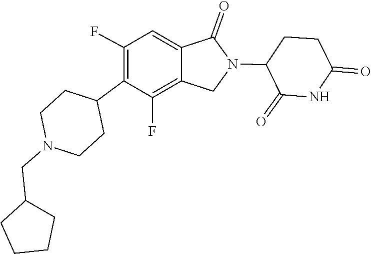 [SMILES: O=C1CCC(N2Cc3c(cc(F)c(C4CCN(CC5CCCC5)CC4)c3F)C2=O)C(=O)N1] | 1 |
| 113 | 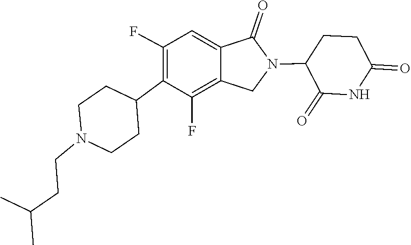 [SMILES: CC(C)CCN1CCC(c2c(F)cc3c(c2F)CN(C2CCC(=O)NC2=O)C3=O)CC1] | 1 |
| 114 | 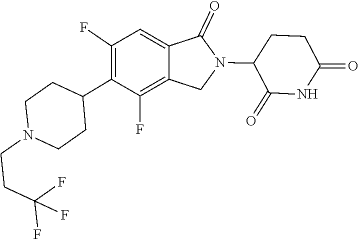 [SMILES: O=C1CCC(N2Cc3c(cc(F)c(C4CCN(CCC(F)(F)F)CC4)c3F)C2=O)C(=O)N1] | 1 |
| 115 | 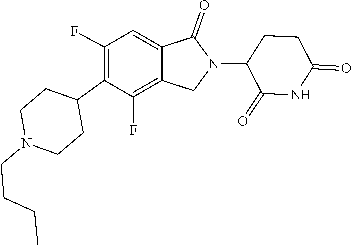 [SMILES: CCCCN1CCC(c2c(F)cc3c(c2F)CN(C2CCC(=O)NC2=O)C3=O)CC1] | 1 |
| 116 | 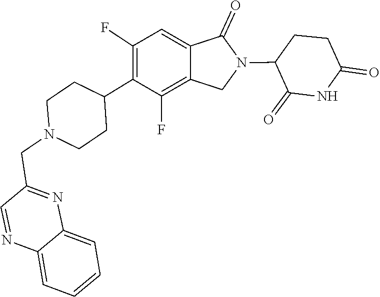 [SMILES: O=C1CCC(N2Cc3c(cc(F)c(C4CCN(Cc5cnc6ccccc6n5)CC4)c3F)C2=O)C(=O)N1] | 1 |
| 117 | 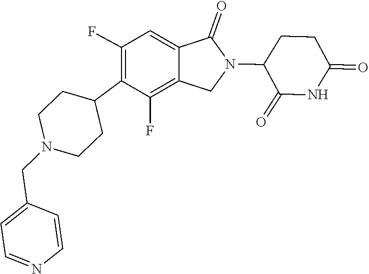 [SMILES: O=C1CCC(N2Cc3c(cc(F)c(C4CCN(Cc5ccncc5)CC4)c3F)C2=O)C(=O)N1] | 1 |
| 118 | 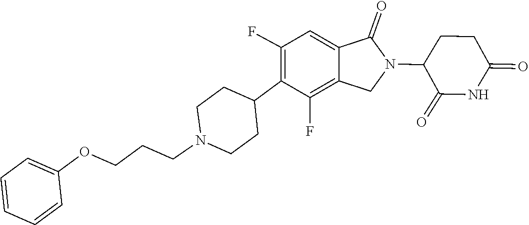 [SMILES: O=C1CCC(N2Cc3c(cc(F)c(C4CCN(CCCOc5ccccc5)CC4)c3F)C2=O)C(=O)N1] | 1 |
| 119 | 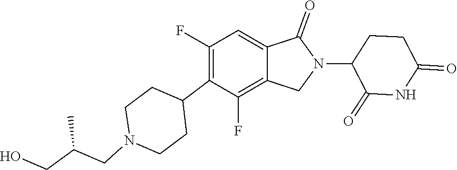 [SMILES: C[C@@H](CO)CN1CCC(c2c(F)cc3c(c2F)CN(C2CCC(=O)NC2=O)C3=O)CC1] | 1 |
| 120 | 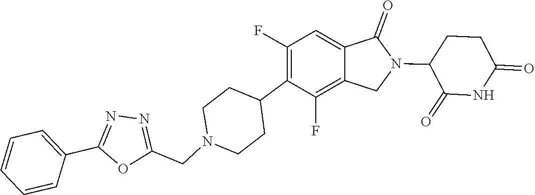 [SMILES: O=C1CCC(N2Cc3c(cc(F)c(C4CCN(Cc5nnc(-c6ccccc6)o5)CC4)c3F)C2=O)C(=O)N1] | 1 |
| 121 | 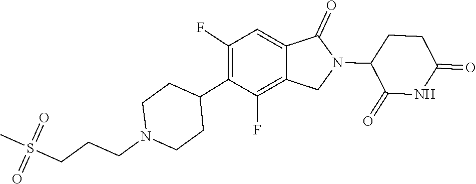 [SMILES: CS(=O)(=O)CCCN1CCC(c2c(F)cc3c(c2F)CN(C2CCC(=O)NC2=O)C3=O)CC1] | 1 |
| 122 | 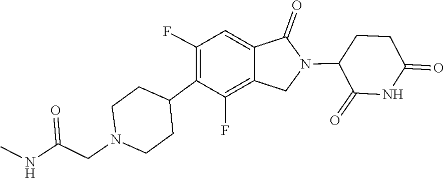 [SMILES: CNC(=O)CN1CCC(c2c(F)cc3c(c2F)CN(C2CCC(=O)NC2=O)C3=O)CC1] | 1 |
| 123 | 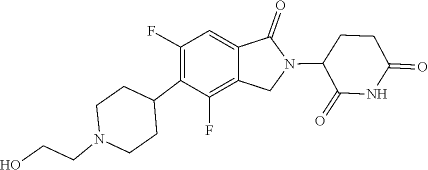 [SMILES: O=C1CCC(N2Cc3c(cc(F)c(C4CCN(CCO)CC4)c3F)C2=O)C(=O)N1] | 1 |
| 124 | 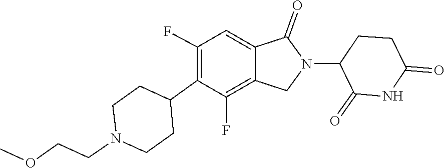 [SMILES: COCCN1CCC(c2c(F)cc3c(c2F)CN(C2CCC(=O)NC2=O)C3=O)CC1] | 1 |
| 125 | 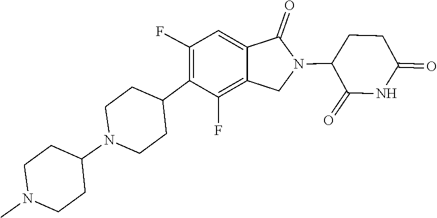 [SMILES: CN1CCC(N2CCC(c3c(F)cc4c(c3F)CN(C3CCC(=O)NC3=O)C4=O)CC2)CC1] | 1 |
| 126 | 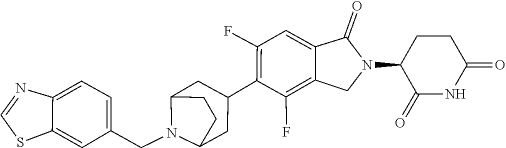 [SMILES: O=C1CC[C@H](N2Cc3c(cc(F)c(C4CC5CCC(C4)N5Cc4ccc5ncsc5c4)c3F)C2=O)C(=O)N1] | 1 |
| 127 | 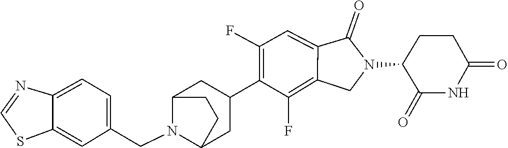 [SMILES: O=C1CC[C@@H](N2Cc3c(cc(F)c(C4CC5CCC(C4)N5Cc4ccc5ncsc5c4)c3F)C2=O)C(=O)N1] | 1 |
| 128 | 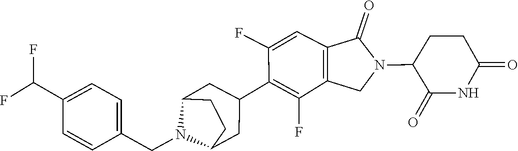 [SMILES: O=C1CCC(N2Cc3c(cc(F)c(C4C[C@H]5CC[C@@H](C4)N5Cc4ccc(C(F)F)cc4)c3F)C2=O)C(=O)N1] | 1 |

### BIOLOGICAL EXAMPLES

### Example B1. Evaluation of Small-Molecule-Induced Degradation of IKZF1 ZNF2_ZNF3 Q1F-Expressing Jurkat Cells

Q1F degron-targeting compounds were screened in Jurkat cells engineered to express a IKZF1 ZNF2_ZNF3 Q1F. The cell line was generated with lentiviral vectors containing CD19 CARs tagged with IKZF1 ZNF2_ZNF3 QIF Nluc, which were transduced into Jurkat cells. The CD19 CAR comprised an anti-CD19 scFv, a CD28 transmembrane domain, a 4-1BB costimulatory domain, a CD3ζ signaling domain, and a ZNF2_ZNF3 Q1F degron (GERPFFCNQC GASFTQKGNL LRHIKLHSGE KPFKCHLCNY ACRRRDALTG HLRTHS; SEQ ID NO: 1; Q1F substitution underlined). Transduced cells were treated with a titration of each small molecule or no drug and then incubated at 37° C. for 18 hours. Cells were washed and stained with appropriate staining reagent to measure CAR levels. The cells were incubated at 4° C. in the staining reagents for 20 mins and then washed 3 times before being read on the flow cytometer. CAR levels were normalized to cells that had not been treated with drug. EC50 and Ymin values were calculated using the resulting titration curve. This identified small molecules that potently degraded the Q1F degron-tagged CARs (Table 1).

### Example B2. Evaluation of Small Molecule Selectivity of Over Aiolos, Ikaros, CK1α, GSPT1 and Helios

DF15 multiple myeloma cells stably expressing ePL-tagged Aiolos, Ikaros, or GSPT1, and MDS-L cells stably expressing ePL-tagged CK1a were generated via lentiviral infection with pLOC-ePL-Aiolos (or Ikaros, GSPT1, or CK1a). The sequences for human Aiolos and Ikaros are shown below:

Human Aiolos:
MEDIQTNAEL KSTQEQSVPA ESAAVLNDYS LTKSHEMENV DSGEGPANED EDIGDDSMKV KDEYSERDEN
VLKSEPMGNA EEPEIPYSYS REYNEYENIK LERHVVSFDS SRPTSGKMNC DVCGLSCISF NVLMVHKRSH
TGERPFQCNQ CGASFTQKGN LLRHIKLHTG EKPFKCHLCN YACQRRDALT GHLRTHSVEK PYKCEFCGRS
YKQRSSLEEH KERCRTFLQS TDPGDTASAE ARHIKAEMGS ERALVLDRLA SNVAKRKSSM PQKFIGEKRH
CFDVNYNSSY MYEKESELIQ TRMMDQAINN AISYLGAEAL RPLVQTPPAP TSEMVPVISS MYPIALTRAE
MSNGAPQELE KKSIHLPEKS VPSERGLSPN NSGHDSTDTD SNHEERQNHI YQQNHMVLSR ARNGMPLLKE
VPRSYELLKP PPICPRDSVK VINKEGEVMD VYRCDHCRVL FLDYVMFTIH MGCHGFRDPF ECNMCGYRSH
DRYEFSSHIA RGEHRALLK (SEQ ID NO: 2; ZNF2 and ZNF3 underlined).
Human Ikaros:
MDADEGQDMS QVSGKESPPV SDTPDEGDEP MPIPEDLSTT SGGQQSSKSD RVVASNVKVE TQSDEENGRA
CEMNGEECAE DLRMLDASGE KMNGSHRDQG SSALSGVGGI RLPNGKLKCD ICGIICIGPN VLMVHKRSHT
GERPFQCNQC GASFTQKGNL LRHIKLHSGE KPFKCHLCNY ACRRRDALTG HLRTHSVGKP HKCGYCGRSY
KQRSSLEEHK ERCHNYLESM GLPGTLYPVI KEETNHSEMA EDLCKIGSER SLVLDRLASN VAKRKSSMPQ
KFLGDKGLSD TPYDSSASYE KENEMMKSHV MDQAINNAIN YLGAESLRPL VQTPPGGSEV VPVISPMYQL
HKPLAEGTPR SNHSAQDSAV ENLLLLSKAK LVPSEREASP SNSCQDSTDT ESNNEEQRSG LIYLTNHIAP
HARNGLSLKE EHRAYDLLRA ASENSQDALR VVSTSGEQMK VYKCEHCRVL FLDHVMYTIH MGCHGFRDPF
ECNMCGYHSQ DRYEFSSHIT RGEHRFHMS (SEQ ID NO: 3; ZNF2 and ZNF3 underlined).

DF15 multiple myeloma cells expressing Ikaros, Aiolos, and GSPT1 fused to an ePL tag (DiscoverX) and MDS-L cells expressing CK1a fused to ePL tag were dispensed into a 384-well plate (Corning no. 3570) prespotted with compounds. Compounds were dispensed by an acoustic dispenser (ATS acoustic transfer system from EDC Biosystems) into a 384-well plate in a 10-point dose response curve using 3-fold dilutions starting at 10 μM and going down to 0.0005 μM. Then, 25 μL of media (RPMI-1640+10% heat inactivated FBS+25 mM Hepes+1 mM Na pyruvate+1×NEAA+1×Pen Strep Glutamine) containing 5000 of DF15 or MSD-L cells was dispensed per well. Assay plates were incubated at 37° C. with 5% CO₂ for 4 hours except 20 hours for GSPT1. After incubation, 25 μL of the InCELL Hunter detection reagent working solution (DiscoverX, catalogue no. 96-0002, Fremont, CA) was added to each well and incubated at room temperature for 60 min protected from light. After 60 min, luminescence was read on an Envision or PHERAstar luminescence reader

For Helios, a stable Jurkat cell line was engineered using CRISPR/Cas9 to insert an in-frame HiBit tag into the carboxy-terminal reading frame of the IKZF2 gene. The sequence of human Helios is shown below:

METEAIDGYI TCDNELSPER EHSNMAIDLT SSTPNGQHAS PSHMTSTNSV KLEMQSDEEC DRKPLSREDE
IRGHDEGSSL EEPLIESSEV ADNRKVQELQ GEGGIRLPNG KLKCDVCGMV CIGPNVLMVH KRSHTGERPF
HCNQCGASFT QKGNLLRHIK LHSGEKPFKC PFCSYACRRR DALTGHLRTH SVGKPHKCNY CGRSYKQRSS
LEEHKERCHN YLQNVSMEAA GQVMSHHVPP MEDCKEQEPI MDNNISLVPF ERPAVIEKLT GNMGKRKSST
PQKFVGEKLM RFSYPDIHFD MNLTYEKEAE LMQSHMMDQA INNAITYLGA EALHPLMQHP PSTIAEVAPV
ISSAYSQVYH PNRIERPISR ETADSHENNM DGPISLIRPK SRPQEREASP SNSCLDSTDS ESSHDDHQSY
QGHPALNPKR KQSPAYMKED VKALDTTKAP KGSLKDIYKV FNGEGEQIRA FKCEHCRVLF LDHVMYTIHM
GCHGYRDPLE CNICGYRSQD RYEFSSHIVR GEHTFH (SEQ ID NO: 4; ZNF2 and ZNF3
underlined).

Test compounds were transferred to 1536 well plates using an acoustic dispenser, and Jurkat/Helios/HiBit cells in DMEM/10% FCS were plated at 10,000 cells/well in a final volume of 5 μL. Cells were incubated at 37 C, 95% RH for 18 hr. Luciferase activity was measured by adding 2 μL/well of Nano-Glo reagent (Promega), incubating at RT for 30 min, and reading luminescence on a microtiter plate reader.

To determine the EC50 value of a compound for the degradation of a given substrate (concentration of compound that achieves half the maximum degradation observed), a four-parameter logistic model (sigmoidal dose-response model) (FIT=(A+{(B−A)/1+[(C/x)D]})) where C is the inflection point (EC50), D is the correlation coefficient, and A and B are the low and high limits of the fit, respectively) was used. All substrate degradation curves were processed and evaluated using ActivityBase (IDBS), a data analysis software package. Ymax is the % protein degraded (Ymin=100−Ymax and is minimum percent protein remaining).

Certain results are shown in Table 8.

TABLE 8

| Compound No. | | Q1F | | |
|---|---|---|---|---|
| (corresponding | Q1F Jurkat | Jurkat- | Helios Jurkat | Helios Jurkat |
| to Table 1) | IC50 (nM) | YMax | Hibit IC50 (μM) | Hibit YMax |
| 1 | 10.366 | 85.34 | ≥10 | nd |
| 2 | 1.434 | 88.79 | ≥10 | nd |
| 3 | 4.369 | 78.69 | ≥10 | nd |
| 4 | 104.583 | 82.28 | ≥10 | nd |
| 5 | 5.702 | 85.84 | ≥10 | nd |
| 6 | 4.997 | 89.10 | ≥10 | nd |
| 7 | 0.265 | 86.21 | ≥10 | nd |
| 8 | 4.971 | 91.70 | ≥10 | nd |
| 9 | 2.741 | 89.11 | ≥10 | nd |
| 10 | 0.202 | 90.68 | ≥10 | nd |
| 11 | 4.911 | 83.49 | ≥10 | nd |
| 12 | 4.332 | 83.37 | ≥10 | nd |
| 13 | 85.993 | 62.41 | ≥10 | nd |
| 14 | 22.440 | 66.94 | ≥10 | nd |
| 15 | 4.939 | 79.90 | ≥10 | nd |
| 16 | 2.665 | 92.89 | 0.0120 | 64.8253 |
| 17 | 28.895 | 69.17 | ≥10 | nd |
| 18 | 0.576 | 93.38 | ≥10 | nd |
| 19 | 5.408 | 83.26 | ≥10 | nd |
| 20 | 33.278 | 77.07 | nd | nd |
| 21 | 40.158 | 70.68 | nd | nd |
| 22 | 14.332 | 80.00 | ≥10 | nd |
| 23 | 278.829 | 51.61 | nd | nd |
| 24 | 100.767 | 57.28 | nd | nd |
| 25 | 48.198 | 67.43 | nd | nd |
| 26 | 6.424 | 78.20 | ≥10 | nd |
| 27 | 17.224 | 76.22 | nd | nd |
| 28 | 4.782 | 79.39 | ≥10 | nd |
| 29 | 6.422 | 87.03 | ≥10 | nd |
| 30 | 191.842 | 57.82 | nd | nd |
| 31 | 14.003 | 82.85 | ≥10 | nd |
| 32 | 9.535 | 80.25 | ≥10 | nd |
| 33 | 192.408 | 60.40 | ≥10 | nd |
| 34 | 7.426 | 91.66 | ≥10 | nd |
| 35 | 144.453 | 63.30 | ≥10 | nd |
| 36 | 3.768 | 77.72 | ≥10 | nd |
| 37 | 33.163 | 62.53 | ≥10 | nd |
| 38 | 24.528 | 70.68 | ≥10 | nd |
| 39 | 93.825 | 58.86 | ≥10 | nd |
| 40 | 67.749 | 60.05 | ≥10 | nd |
| 41 | 95.301 | 66.15 | ≥10 | nd |
| 42 | 4.392 | 87.69 | ≥10 | nd |
| 43 | 2.749 | 80.21 | ≥10 | nd |
| 44 | 120.354 | 82.07 | ≥10 | nd |
| 45 | 0.343 | 90.57 | ≥10 | nd |
| 46 | 0.112 | 91.79 | 6,518.8086 | 50.8729 |
| 47 | 2.504 | 88.80 | ≥10 | nd |
| 48 | 2.239 | 87.79 | ≥10 | nd |
| 49 | 0.108 | 91.02 | ≥10 | nd |
| 50 | 1.357 | 88.41 | ≥10 | nd |
| 51 | 0.097 | 91.96 | ≥10 | nd |
| 52 | 0.910 | 91.75 | ≥10 | nd |
| 53 | 0.727 | 93.28 | ≥10 | nd |
| 54 | 0.114 | 93.29 | ≥10 | nd |
| 55 | 0.468 | 88.66 | ≥10 | nd |
| 56 | 0.571 | 84.42 | ≥10 | nd |
| 57 | 100.644 | 63.25 | ≥10 | nd |
| 58 | 10.734 | 86.26 | ≥10 | nd |
| 59 | 0.010 | 92.23 | ≥10 | nd |
| 60 | 0.691 | 91.78 | ≥10 | nd |
| 61 | 0.204 | 93.40 | 0.1515 | 59.5442 |
| 62 | 0.168 | 92.63 | ≥10 | nd |
| 63 | 0.103 | 92.97 | 3.3066 | 55.0199 |
| 64 | 2.109 | 92.92 | ≥10 | nd |
| 65 | 1.093 | 85.45 | ≥10 | nd |
| 66 | 46.117 | 74.94 | ≥10 | nd |
| 67 | 0.563 | 88.95 | ≥10 | nd |
| 68 | 50.523 | 63.46 | ≥10 | nd |
| 69 | 35.174 | 53.90 | ≥10 | nd |
| 70 | 15.042 | 95.49 | ≥10 | nd |
| 71 | 23.709 | 95.69 | ≥10 | nd |
| 72 | 21.222 | 92.52 | ≥10 | nd |
| 73 | 3.113 | 95.31 | ≥10 | nd |
| 74 | 107.816 | 74.91 | ≥10 | nd |
| 75 | 3.325 | 89.04 | 0.0651 | 65.5325 |
| 76 | 4.885 | 88.79 | 0.0063 | 72.1901 |
| 77 | 14.754 | 94.78 | ≥10 | nd |
| 78 | 24.528 | 61.72 | ≥10 | nd |
| 79 | 162.357 | 60.01 | ≥10 | nd |
| 80 | 0.533 | 85.09 | ≥10 | nd |
| 81 | 3.055 | 93.04 | ≥10 | nd |
| 82 | 1.724 | 88.23 | ≥10 | nd |
| 83 | 19.868 | 76.77 | ≥10 | nd |
| 84 | 2.095 | 90.29 | ≥10 | nd |
| 85 | 1.534 | 89.31 | ≥10 | nd |
| 86 | 1.940 | 92.08 | ≥10 | nd |
| 87 | 4.060 | 94.20 | ≥10 | nd |
| 88 | 3.099 | 91.59 | ≥10 | nd |
| 89 | 2.668 | 94.19 | ≥10 | nd |
| 90 | 4.685 | 93.13 | ≥10 | nd |
| 91 | 3.902 | 92.07 | ≥10 | nd |
| 92 | 9.159 | 63.19 | ≥10 | nd |
| 93 | 217.167 | 69.07 | ≥10 | nd |
| 94 | 655.496 | 60.92 | ≥10 | nd |
| 95 | 10.484 | 84.50 | ≥10 | nd |
| 96 | 4.171 | 89.72 | ≥10 | nd |
| 97 | 3.880 | 88.33 | ≥10 | nd |
| 98 | 39.832 | 76.94 | ≥10 | nd |
| 99 | 369.038 | 58.62 | ≥10 | nd |
| 100 | 22.607 | 75.48 | ≥10 | nd |
| 101 | 0.473 | 86.86 | ≥10 | nd |
| 102 | 0.283 | 84.34 | ≥10 | nd |
| 103 | 1.962 | 84.72 | ≥10 | nd |
| 104 | 30.897 | 83.59 | ≥10 | nd |
| 105 | 83.461 | 75.33 | ≥10 | nd |
| 106 | 0.698 | 91.76 | ≥10 | nd |
| 107 | 2.102 | 89.72 | ≥10 | nd |
| 108 | 3.657 | 83.06 | ≥10 | nd |
| 109 | 2.276 | 91.49 | ≥10 | nd |
| 110 | 1.118 | 90.46 | ≥10 | nd |
| 111 | 7.451 | 82.68 | ≥10 | nd |
| 112 | 3.226 | 84.61 | ≥10 | nd |
| 113 | 2.725 | 82.50 | ≥10 | nd |
| 114 | 10.839 | 74.85 | ≥10 | nd |
| 115 | 6.662 | 79.69 | ≥10 | nd |
| 116 | 3.187 | 85.51 | ≥10 | nd |
| 117 | 6.258 | 86.12 | ≥10 | nd |
| 118 | 2.863 | 83.02 | ≥10 | nd |
| 119 | 8.653 | 80.74 | ≥10 | nd |
| 120 | 165.329 | 65.83 | ≥10 | nd |
| 121 | 20.385 | 76.67 | ≥10 | nd |
| 122 | 65.090 | 74.21 | ≥10 | nd |
| 123 | 21.263 | 74.48 | ≥10 | nd |
| 124 | 11.016 | 76.65 | ≥10 | nd |
| 125 | 22.098 | 80.06 | ≥10 | nd |
| 126 | 0.427 | 92.25 | ≥10 | nd |
| 127 | 5.944 | 91.24 | ≥10 | nd |
| 128 | 17.393 | 87.15 | ≥10 | nd |

* nd = not determined.

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 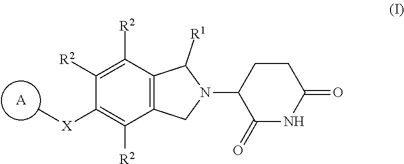
or a pharmaceutically acceptable salt thereof, wherein:
R¹ is H or oxo;
each R² is independently H or halo, wherein at least one R² is fluoro;
X is bond, C₁-C₃ alkylene, —C(O)NHCH₂—, —NHC(O)—, —C(O)—, or —(C₁-C₃ alkylene)NH(C₁-C₃ alkylene)-;
Ring A is optionally substituted C₅-C₆ cycloalkyl, optionally substituted C₅-C₆ aryl, optionally substituted 6-10 membered heterocyclyl, or optionally substituted 5-9 membered heteroaryl, wherein heterocyclyl or heteroaryl contains 1-3 heteroatoms selected from nitrogen, oxygen, and sulfur.

2. The compound of claim 1, or a pharmaceutically acceptable salt thereof, wherein:
the compound of Formula (I) is the compound of Formula (Ia) or Formula (Ib):

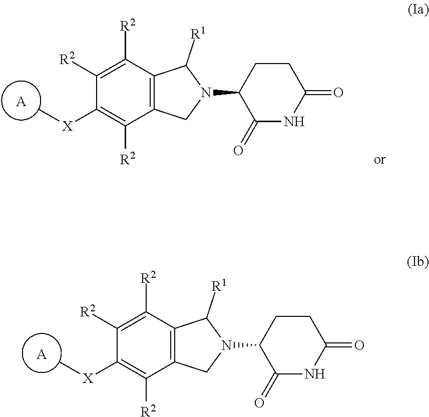

3-5. (canceled)

6. The compound of claim 1, or a pharmaceutically acceptable salt thereof, wherein:
at least one R² is H.

7. The compound of claim 1, or a pharmaceutically acceptable salt thereof, wherein:
one or two R² is F, Cl, or Br.

8-14. (canceled)

15. The compound of claim 1, or a pharmaceutically acceptable salt thereof, wherein:
Ring A is optionally substituted cyclohexyl or optionally substituted C₆ aryl.

16. The compound of claim 15, or a pharmaceutically acceptable salt thereof, wherein:
Ring A is

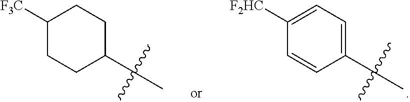 [SMILES: *C1CCC(C(F)(F)F)CC1 ; *c1ccc(C(F)F)cc1]

17-18. (canceled)

19. The compound of claim 1, or a pharmaceutically acceptable salt thereof, wherein:
Ring A is optionally substituted 6-10 membered heterocyclyl that contains 1-3 heteroatoms selected from nitrogen, oxygen, and sulfur.

20. The compound of claim 19, or a pharmaceutically acceptable salt thereof, wherein:
Ring A is 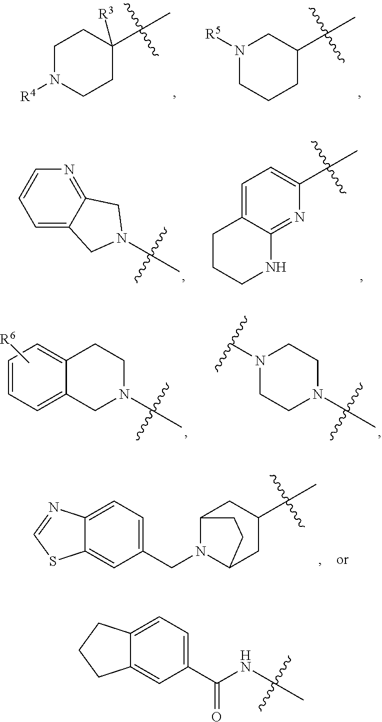
wherein R³ is H or OH; R⁴ is optionally substituted C₁-C₃ alkyl, optionally substituted C₃-C₆ cycloalkyl, —C(O)(6-membered heteroaryl), or —C(O)(9-membered heterocyclyl); R⁵ is optionally substituted C₁-C₃ alkyl; and R⁶ is cyano or halo.

21. The compound of claim 20, or a pharmaceutically acceptable salt thereof, wherein:
Ring A is

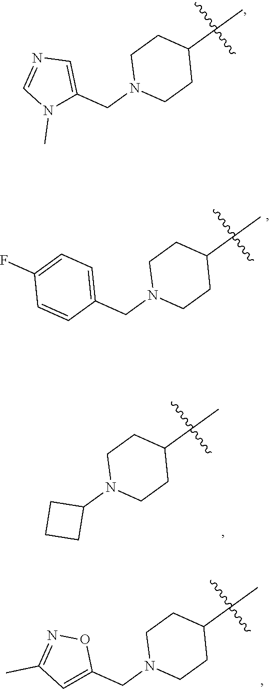 [SMILES: *C1CCN(Cc2cncn2C)CC1 ; *C1CCN(Cc2ccc(F)cc2)CC1 ; *C1CCN(C2CCC2)CC1 ; *C1CCN(Cc2cc(C)no2)CC1]

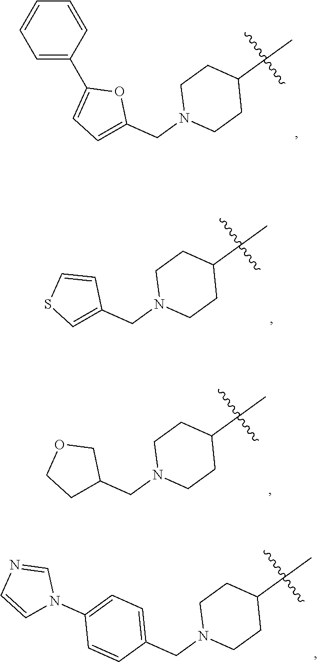 [SMILES: *C1CCN(Cc2ccc(-c3ccccc3)o2)CC1 ; *C1CCN(Cc2ccsc2)CC1 ; *C1CCN(CC2CCOC2)CC1 ; *C1CCN(Cc2ccc(-n3ccnc3)cc2)CC1]

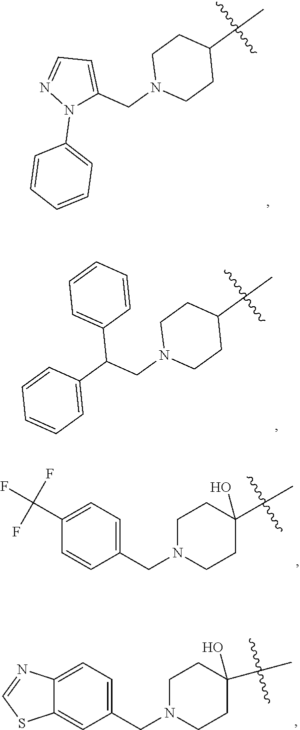

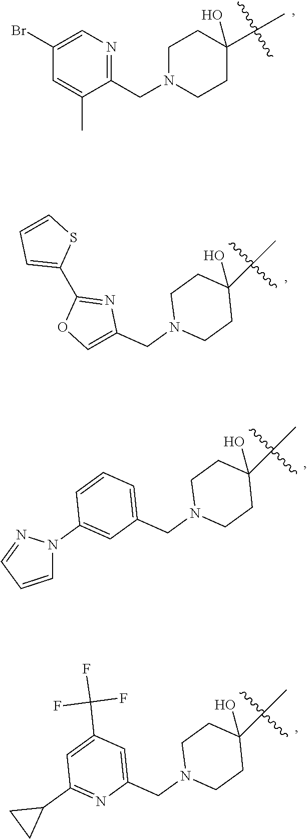 [SMILES: *C1(O)CCN(Cc2ncc(Br)cc2C)CC1 ; *C1(O)CCN(Cc2coc(-c3cccs3)n2)CC1 ; *C1(O)CCN(Cc2cccc(-n3cccn3)c2)CC1 ; *C1(O)CCN(Cc2cc(C(F)(F)F)cc(C3CC3)n2)CC1]

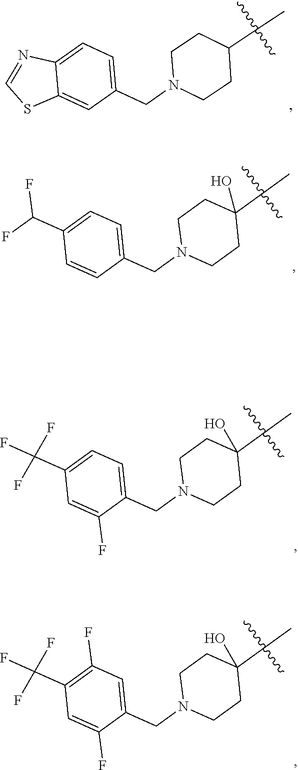 [SMILES: *C1CCN(Cc2ccc3ncsc3c2)CC1 ; *C1(O)CCN(Cc2ccc(C(F)F)cc2)CC1 ; *C1(O)CCN(Cc2ccc(C(F)(F)F)cc2F)CC1 ; *C1(O)CCN(Cc2cc(F)c(C(F)(F)F)cc2F)CC1]

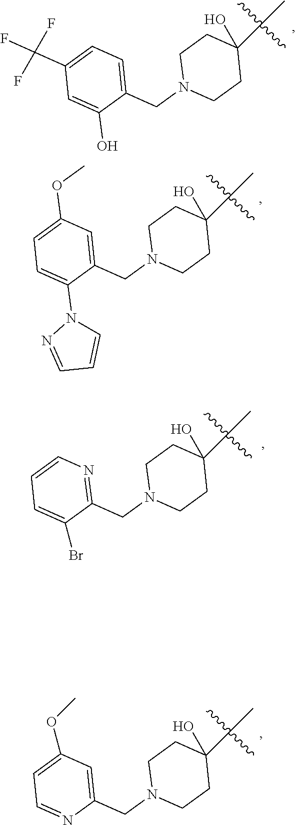

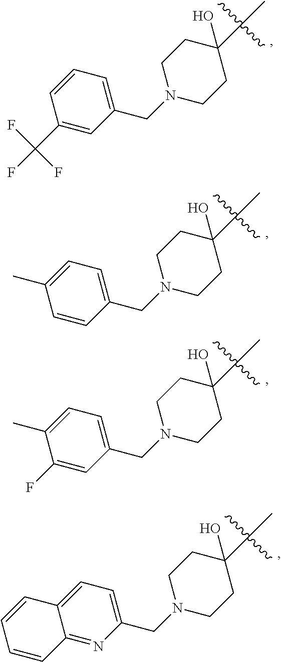 [SMILES: *C1(O)CCN(Cc2cccc(C(F)(F)F)c2)CC1 ; *C1(O)CCN(Cc2ccc(C)cc2)CC1 ; *C1(O)CCN(Cc2ccc(C)c(F)c2)CC1 ; *C1(O)CCN(Cc2ccc3ccccc3n2)CC1]

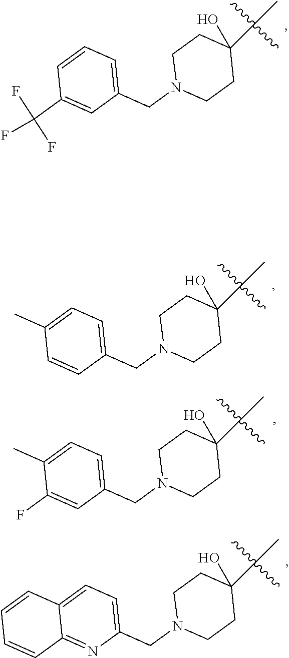 [SMILES: *C1(O)CCN(Cc2cccc(C(F)(F)F)c2)CC1 ; *C1(O)CCN(Cc2ccc(C)cc2)CC1 ; *C1(O)CCN(Cc2ccc(C)c(F)c2)CC1 ; *C1(O)CCN(Cc2ccc3ccccc3n2)CC1]

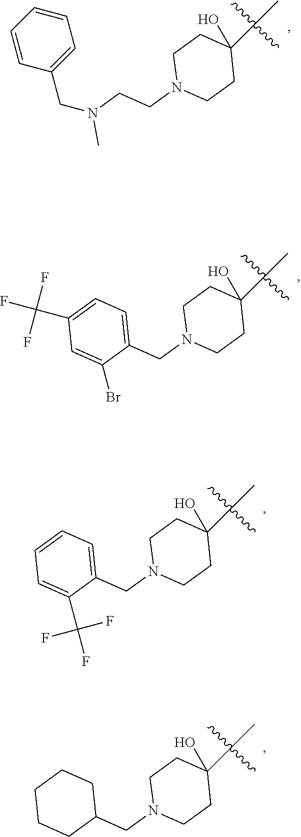 [SMILES: *C1(O)CCN(CCN(C)Cc2ccccc2)CC1 ; *C1(O)CCN(Cc2ccc(C(F)(F)F)cc2Br)CC1 ; *C1(O)CCN(Cc2ccccc2C(F)(F)F)CC1 ; *C1(O)CCN(CC2CCCCC2)CC1]

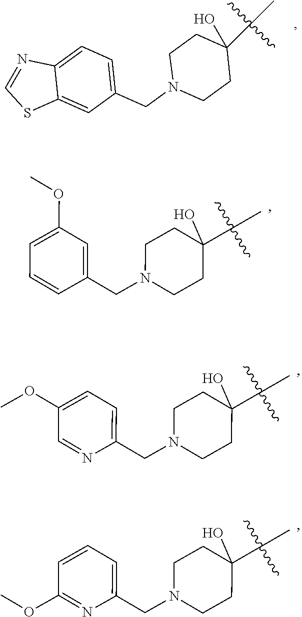 [SMILES: *C1(O)CCN(Cc2ccc3ncsc3c2)CC1 ; *C1(O)CCN(Cc2cccc(OC)c2)CC1 ; *C1(O)CCN(Cc2ccc(OC)cn2)CC1 ; *C1(O)CCN(Cc2cccc(OC)n2)CC1]

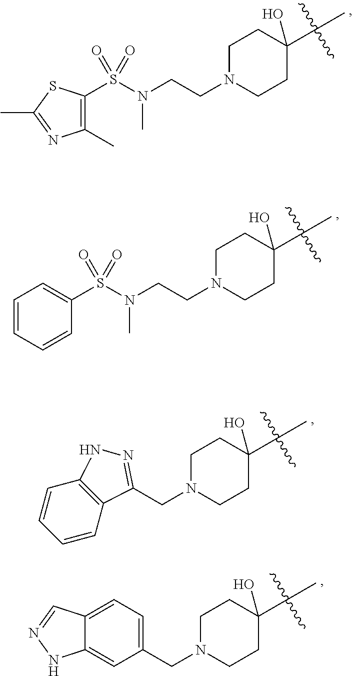 [SMILES: *C1(O)CCN(CCN(C)S(=O)(=O)c2sc(C)nc2C)CC1 ; *C1(O)CCN(CCN(C)S(=O)(=O)c2ccccc2)CC1 ; *C1(O)CCN(Cc2n[nH]c3ccccc23)CC1 ; *C1(O)CCN(Cc2ccc3cn[nH]c3c2)CC1]

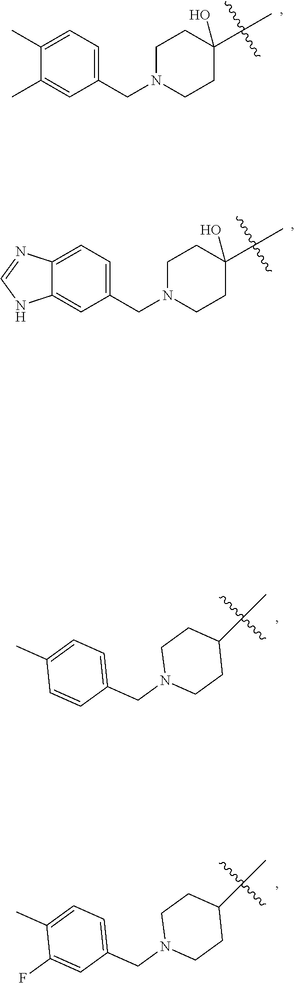 [SMILES: *C1(O)CCN(Cc2ccc(C)c(C)c2)CC1 ; *C1(O)CCN(Cc2ccc3nc[nH]c3c2)CC1 ; *C1CCN(Cc2ccc(C)cc2)CC1 ; *C1CCN(Cc2ccc(C)c(F)c2)CC1]

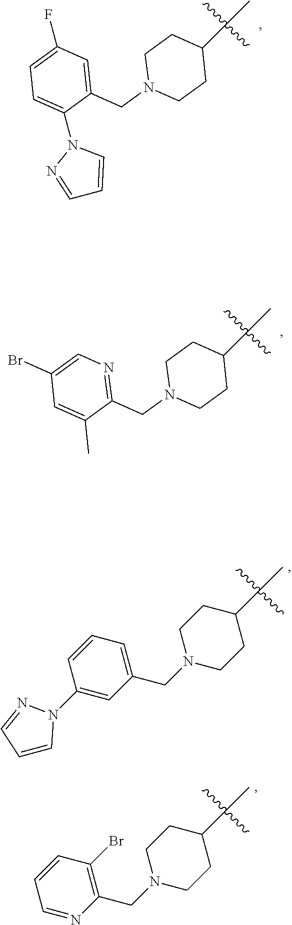 [SMILES: *C1CCN(Cc2cc(F)ccc2-n2cccn2)CC1 ; *C1CCN(Cc2ncc(Br)cc2C)CC1 ; *C1CCN(Cc2cccc(-n3cccn3)c2)CC1 ; *C1CCN(Cc2ncccc2Br)CC1]

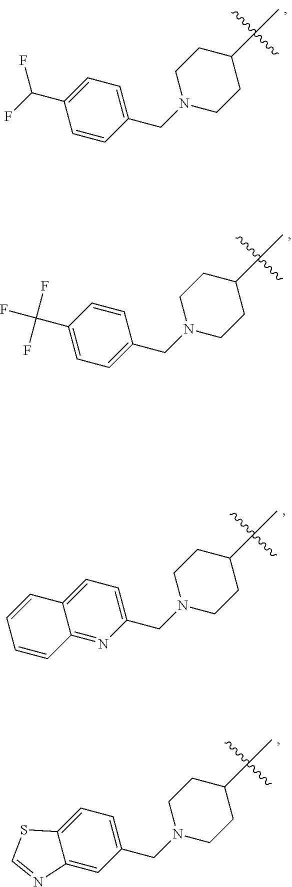 [SMILES: *C1CCN(Cc2ccc(C(F)F)cc2)CC1 ; *C1CCN(Cc2ccc(C(F)(F)F)cc2)CC1 ; *C1CCN(Cc2ccc3ccccc3n2)CC1 ; *C1CCN(Cc2ccc3scnc3c2)CC1]

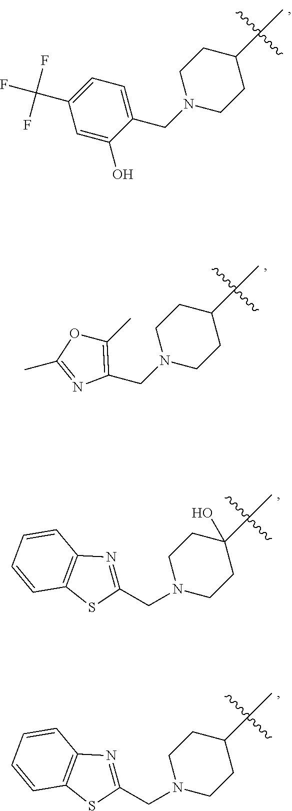 [SMILES: *C1CCN(Cc2ccc(C(F)(F)F)cc2O)CC1 ; *C1CCN(Cc2nc(C)oc2C)CC1 ; *C1(O)CCN(Cc2nc3ccccc3s2)CC1 ; *C1CCN(Cc2nc3ccccc3s2)CC1]

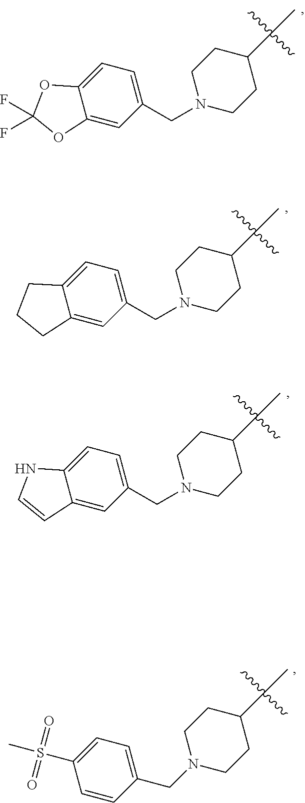 [SMILES: *C1CCN(Cc2ccc3c(c2)OC(F)(F)O3)CC1 ; *C1CCN(Cc2ccc3c(c2)CCC3)CC1 ; *C1CCN(Cc2ccc3[nH]ccc3c2)CC1 ; *C1CCN(Cc2ccc(S(C)(=O)=O)cc2)CC1]

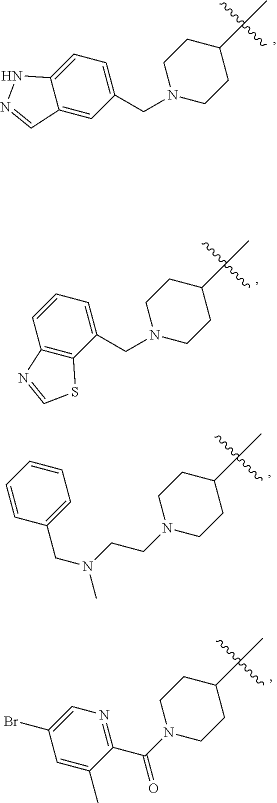 [SMILES: *C1CCN(Cc2ccc3[nH]ncc3c2)CC1 ; *C1CCN(Cc2cccc3ncsc23)CC1 ; *C1CCN(CCN(C)Cc2ccccc2)CC1 ; *C1CCN(C(=O)c2ncc(Br)cc2C)CC1]

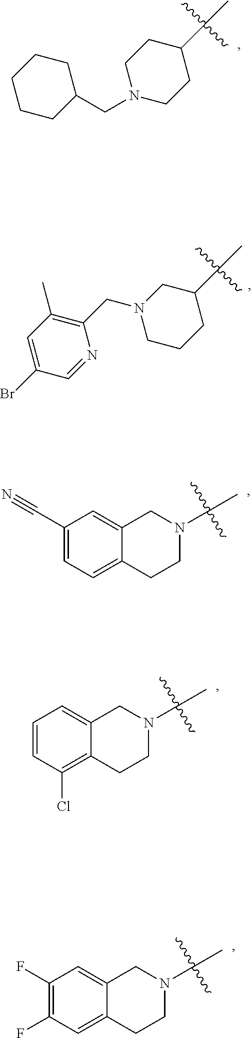 [SMILES: *C1CCN(CC2CCCCC2)CC1 ; *C1CCCN(Cc2ncc(Br)cc2C)C1 ; *N1CCc2ccc(C#N)cc2C1 ; *N1CCc2c(Cl)cccc2C1 ; *N1CCc2cc(F)c(F)cc2C1]

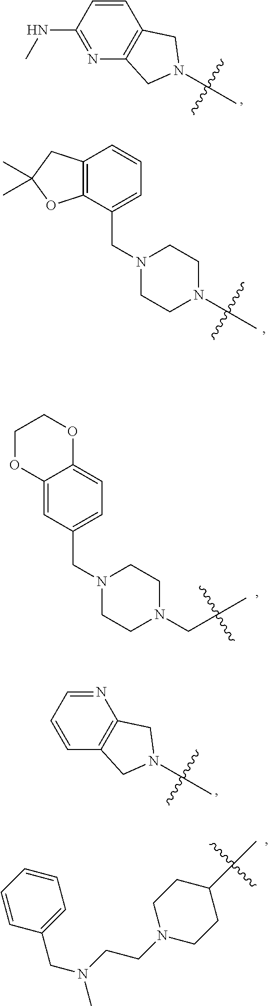 [SMILES: *N1Cc2ccc(NC)nc2C1 ; *N1CCN(Cc2cccc3c2OC(C)(C)C3)CC1 ; *CN1CCN(Cc2ccc3c(c2)OCCO3)CC1 ; *N1Cc2cccnc2C1 ; *C1CCN(CCN(C)Cc2ccccc2)CC1]

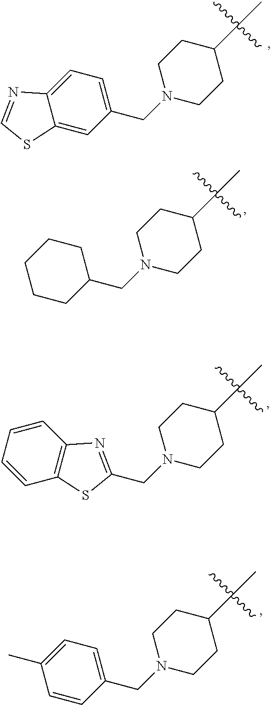 [SMILES: *C1CCN(Cc2ccc3ncsc3c2)CC1 ; *C1CCN(CC2CCCCC2)CC1 ; *C1CCN(Cc2nc3ccccc3s2)CC1 ; *C1CCN(Cc2ccc(C)cc2)CC1]

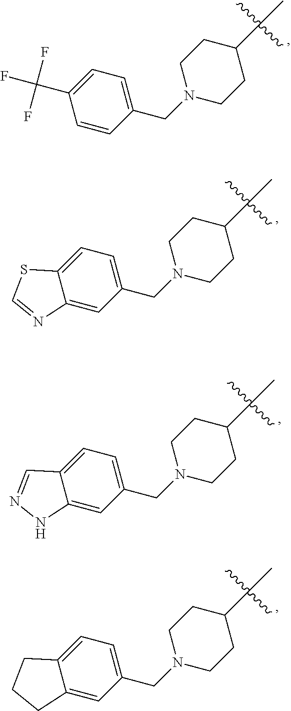 [SMILES: *C1CCN(Cc2ccc(C(F)(F)F)cc2)CC1 ; *C1CCN(Cc2ccc3scnc3c2)CC1 ; *C1CCN(Cc2ccc3cn[nH]c3c2)CC1 ; *C1CCN(Cc2ccc3c(c2)CCC3)CC1]

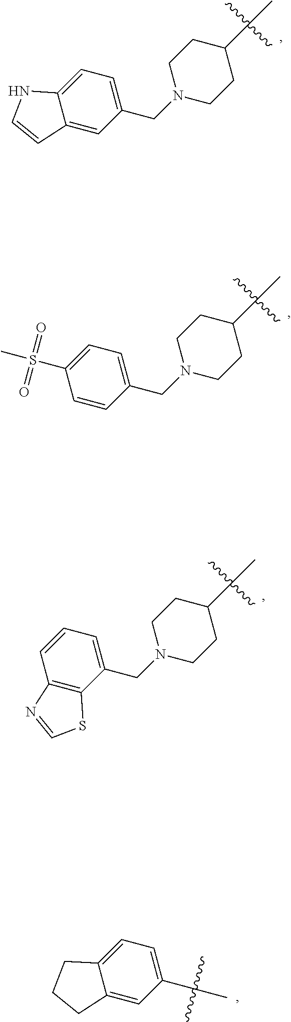 [SMILES: *C1CCN(Cc2ccc3[nH]ccc3c2)CC1 ; *C1CCN(Cc2ccc(S(C)(=O)=O)cc2)CC1 ; *C1CCN(Cc2cccc3ncsc23)CC1 ; *c1ccc2c(c1)CCC2]

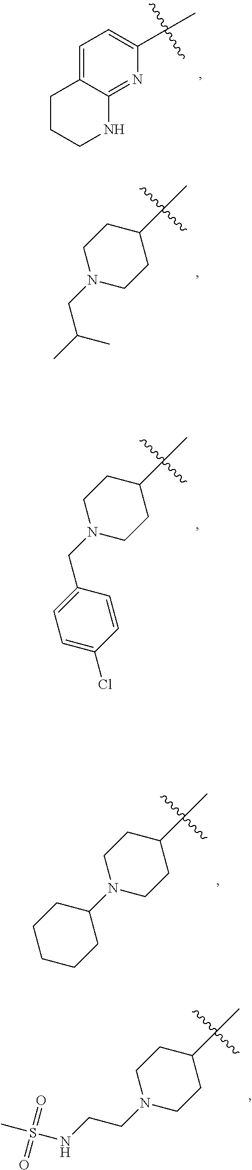 [SMILES: *c1ccc2c(n1)NCCC2 ; *C1CCN(CC(C)C)CC1 ; *C1CCN(Cc2ccc(Cl)cc2)CC1 ; *C1CCN(C2CCCCC2)CC1 ; *C1CCN(CCNS(C)(=O)=O)CC1]

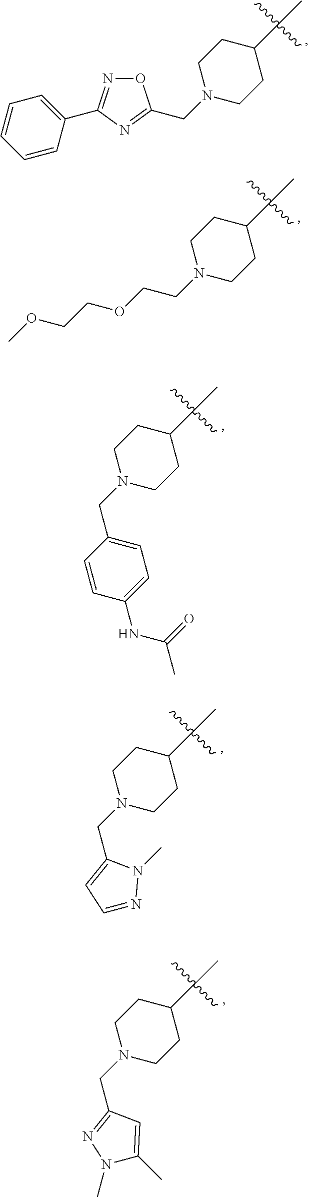 [SMILES: *C1CCN(Cc2nc(-c3ccccc3)no2)CC1 ; *C1CCN(CCOCCOC)CC1 ; *C1CCN(Cc2ccc(NC(C)=O)cc2)CC1 ; *C1CCN(Cc2ccnn2C)CC1 ; *C1CCN(Cc2cc(C)n(C)n2)CC1]

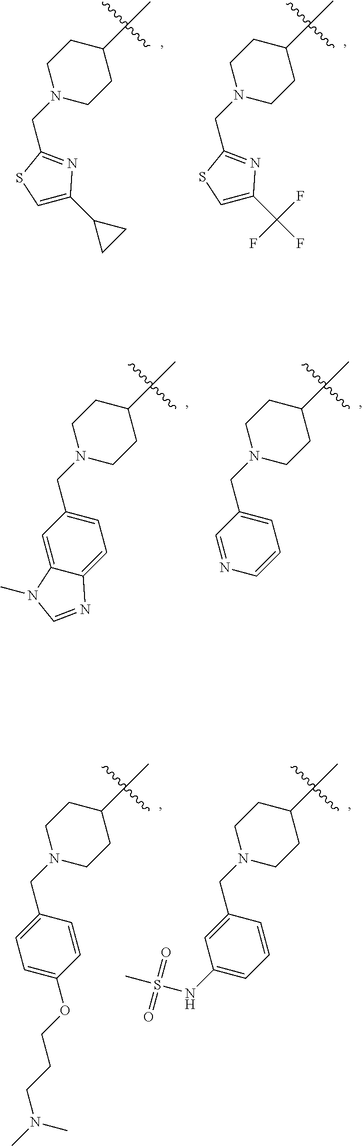 [SMILES: *C1CCN(Cc2nc(C3CC3)cs2)CC1 ; *C1CCN(Cc2nc(C(F)(F)F)cs2)CC1 ; *C1CCN(Cc2ccc3ncn(C)c3c2)CC1 ; *C1CCN(Cc2cccnc2)CC1 ; *C1CCN(Cc2ccc(OCCCN(C)C)cc2)CC1 ; *C1CCN(Cc2cccc(NS(C)(=O)=O)c2)CC1]

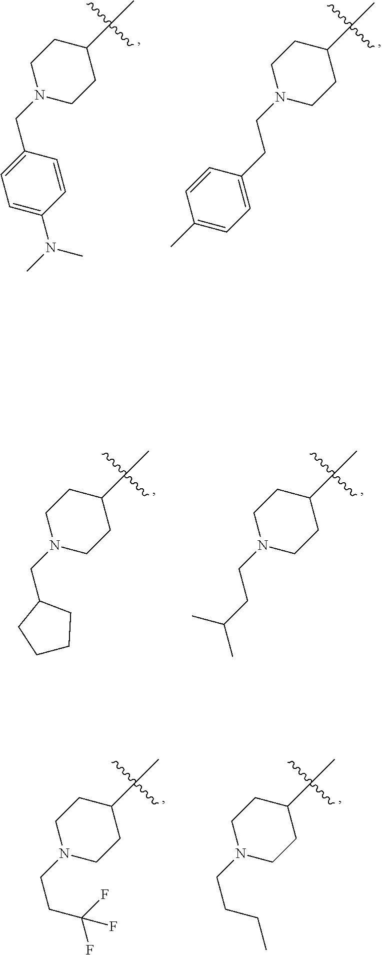 [SMILES: *C1CCN(Cc2ccc(N(C)C)cc2)CC1 ; *C1CCN(CCc2ccc(C)cc2)CC1 ; *C1CCN(CC2CCCC2)CC1 ; *C1CCN(CCC(C)C)CC1 ; *C1CCN(CCC(F)(F)F)CC1 ; *C1CCN(CCCC)CC1]

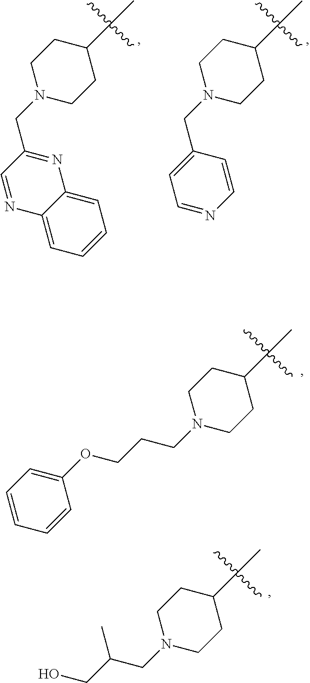 [SMILES: *C1CCN(Cc2cnc3ccccc3n2)CC1 ; *C1CCN(Cc2ccncc2)CC1 ; *C1CCN(CCCOc2ccccc2)CC1 ; *C1CCN(CC(C)CO)CC1]

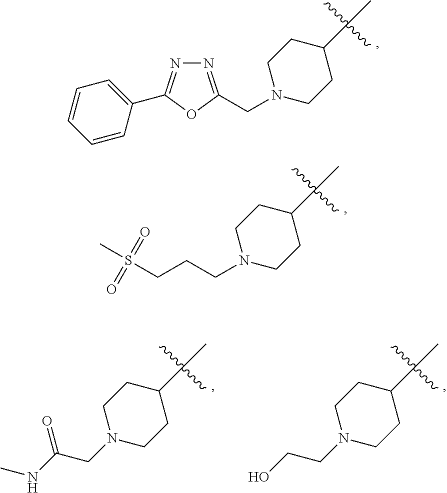 [SMILES: *C1CCN(Cc2nnc(-c3ccccc3)o2)CC1 ; *C1CCN(CCCS(C)(=O)=O)CC1 ; *C1CCN(CC(=O)NC)CC1 ; *C1CCN(CCO)CC1]

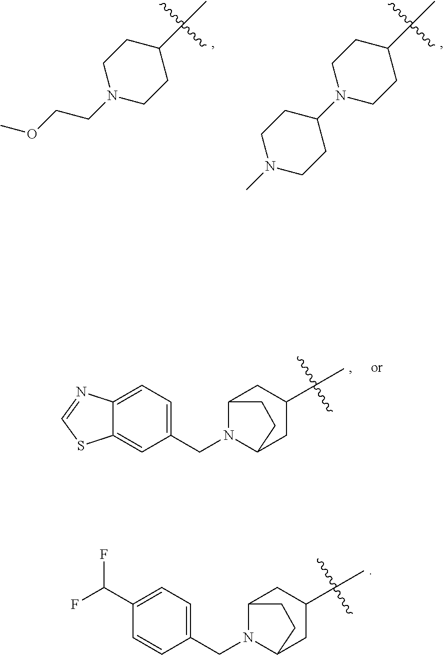 [SMILES: *C1CCN(CCOC)CC1 ; *C1CCN(C2CCN(C)CC2)CC1 ; *C1CC2CCC(C1)N2Cc1ccc2ncsc2c1 ; *C1CC2CCC(C1)N2Cc1ccc(C(F)F)cc1]

22. The compound of claim 21, wherein: 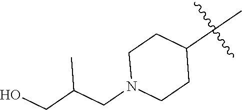 [SMILES: *C1CCN(CC(C)CO)CC1]
is

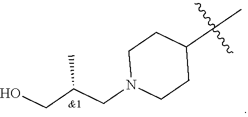 [SMILES: *C1CCN(C[C@@H](C)CO)CC1]

23. The compound of claim 21, wherein: 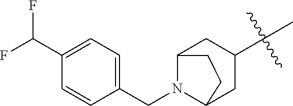 [SMILES: *C1CC2CCC(C1)N2Cc1ccc(C(F)F)cc1]
is

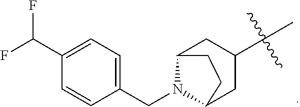 [SMILES: *C1C[C@H]2CC[C@@H](C1)N2Cc1ccc(C(F)F)cc1]

24. The compound of claim 1, or a pharmaceutically acceptable salt thereof, wherein:
Ring A is optionally substituted 5-9 membered heteroaryl that contains 1-3 heteroatoms selected from nitrogen, oxygen, and sulfur.

25. The compound of claim 24, or a pharmaceutically acceptable salt thereof, wherein:
Ring A is 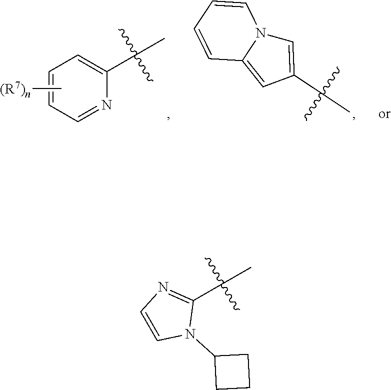
wherein R⁷ is optionally substituted C₁-C₃ alkyl or optionally substituted amine and n is 0, 1, 2, 3, or 4.

26. The compound of claim 25, or a pharmaceutically acceptable salt thereof, wherein: 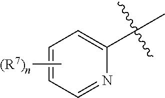
is

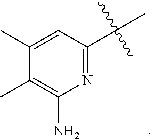 [SMILES: *c1cc(C)c(C)c(N)n1]

27. The compound of claim 1, or a pharmaceutically acceptable salt thereof, wherein the compound is Formula (IIa), Formula (IIb), Formula (IIc), or Formula (IId):

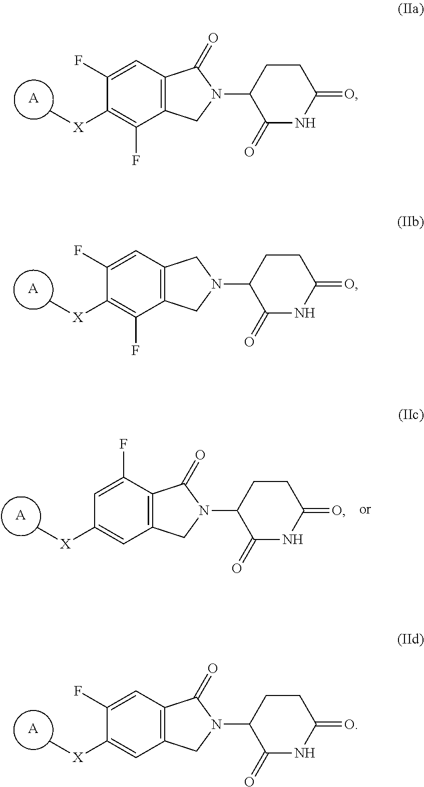

28-30. (canceled)

31. The compound of claim 1, or a pharmaceutically acceptable salt thereof, wherein the compound is Formula (IIIa), Formula (IIIb), Formula (IIIc), or Formula (IIId): 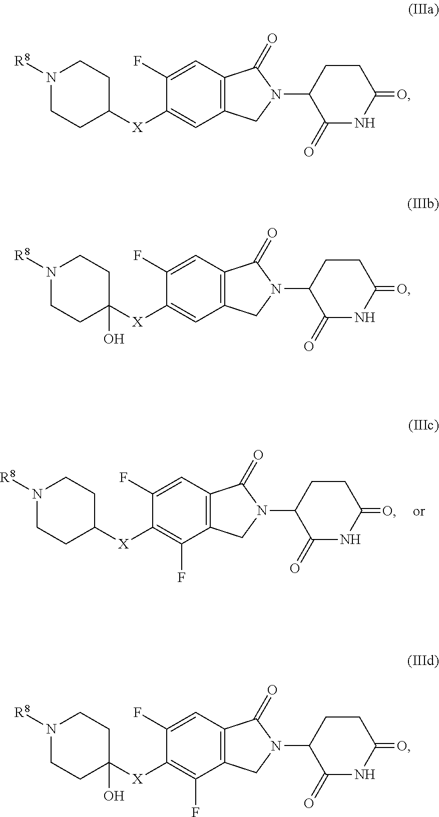
wherein R⁸ is optionally substituted C₁-C₆ alkyl or optionally substituted C₃-C₆ cycloalkyl.

32-34. (canceled)

35. A compound selected from the compounds of Table 1, Table 2, Table 3, Table 4, Table 5, Table 6, and Table 7 or a pharmaceutically acceptable salt thereof.

36. A pharmaceutical composition comprising the compound of claim 1, or a pharmaceutically acceptable salt thereof, and a pharmaceutically acceptable excipient.

37. A method of reducing the level of an engineered polypeptide in a cell, comprising contacting the cell with an effective amount of the compound of claim 1, or a pharmaceutically acceptable salt thereof, or the pharmaceutical composition of claim 36.

38-42. (canceled)

43. A method of reducing the level of an engineered polypeptide in a cell in a subject, comprising administering to the subject an effective amount of the compound of claim 1, or a pharmaceutically acceptable salt thereof, or the pharmaceutical composition of claim 36.

44-50. (canceled)
